(19) United States
(12) Patent Application Publication
(10) Pub. No.: US 2025/0251406 A1
(43) Pub. Date: Aug. 7, 2025

(54) BIOMARKER-BASED TREATMENT AND DIAGNOSTIC METHODS FOR IL-17-DEPENDENT CONDITIONS

(71) Applicant: MOONLAKE IMMUNOTHERAPEUTICS AG, Zug (CH)

(72) Inventors: Kristian REICH, Hamburg (DE); Eva CULLEN, Brussels (BE); Sara Raquel SOUTO PEREIRA, Porto (PT); Alex GODWOOD, Cambridge (GB); Jorge SANTOS DA SILVA, Zug (CH)

(73) Assignee: MOONLAKE IMMUNOTHERAPEUTICS AG, Zug (CH)

(21) Appl. No.: 19/187,615

(22) Filed: Apr. 23, 2025

Related U.S. Application Data
(63) Continuation of application No. 19/122,557, filed on Jan. 1, 0001.
(60) Provisional application No. 63/460,230, filed on Apr. 18, 2023.
(60) Provisional application No. 63/460,240, filed on Apr. 18, 2023.

(51) Int. Cl. G01N 33/68; C07K 16/24; C12Q 1/6883

(52) U.S. Cl. CPC G01N 33/6869; C07K 16/244; C12Q 1/6883; C12Q 2600/106; C12Q 2600/158; G01N 2333/522; G01N 2333/523; G01N 2333/5412; G01N 2333/545; G01N 2333/55; G01N 2800/20; G01N 2800/52

(57) ABSTRACT

Biomarker-based treatment, monitoring, and diagnostic methods for IL-17 dependent conditions, including hidradenitis suppurativa, are disclosed. The biomarkers may be selected from IL6, PLA2G2A, IL19, P13, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, CXCL13, PCDH1, BOC, MEPE, ADAM 23, THOP1, IL1RL2, RCOR1, EDAR, and combinations thereof. Kits for measuring the biomarkers, diagnosing and treating IL-17-dependent conditions, and identifying super-responders are also disclosed.

### CROSS-REFERENCE TO RELATED APPLICATIONS

The instant application is a continuation of U.S. Application Ser. No. 19/122,557, which is a U.S. National Stage entry of PCT/IB2024/053796, filed Apr. 18, 2024, which claims the benefit of U.S. Provisional Application No. 63/460,240 filed Apr. 18, 2023, as well as U.S. Provisional Application No. 63/460,230, filed Apr. 18, 2023. The disclosure of each of the above applications is expressly incorporated by reference herein in its entirety.

### INCORPORATION BY REFERENCE STATEMENT REGARDING SEQUENCE LISTING

The instant application contains a Sequence Listing which has been submitted electronically in XML file format and is hereby incorporated by reference in its entirety. Said XML copy, created on Apr. 23, 2025, is named “P68859_SL.xml” and is 92,300 bytes in size.

### BACKGROUND

Biomarkers for various immune-mediated diseases have been disclosed. For example, WO 2019/143585 discloses detecting the presence of IL-19 in a sample from a patient having an immune-related disease such as psoriasis, atopic dermatitis, or diabetic neuropathy and treating the patient with an IL-19 antibody if the IL-19 is detected above a reference value.

As another example, WO 2014/100312 discloses prospectively selecting psoriasis patients likely to benefit from treatment with antagonists of IL-23 based on the presence of one or more single nucleotide polymorphisms in the genome. WO 2012/093254 relates to the use of lipocalin 2 (LCN2) as a biomarker as well as use of an anti-IL-17A antibody to reduce LCN2 expression in an animal model of multiple sclerosis. However, there remains a need for more personalized treatment regimes as well as a need to identify patients with immune-related diseases earlier and more accurately evaluate the severity of these diseases, particularly IL-17-dependent conditions, to manage disease progression more effectively and/or relapse post treatment. There also remains a need to identify specific patients and/or patient populations among those meeting standard diagnostic criteria who would positively respond to treatment with any particular therapeutic agent.

Historically, IL-17 was used to describe what later became clear to represent just one member of the IL-17 family of cytokines. Those of ordinary skill, therefore, assumed that IL-17A was the primary and main pro-inflammatory signal driving immune conditions related to the IL-17 pathway such as psoriasis, psoriatic arthritis, and axial spondyloarthropathies. Non-communicable inflammatory skin diseases characterized by exaggerated IL-17 activity have also been termed Type 3 diseases (Nakamura et al., Curr Rheumatol Rep. 23(5):31 (2021); Eyerich et al., J Eur Acad Dermatol Venereol. 32(5):692-703 (2018); Annunziato et al., 135(3):626-35 (2015)), and over the years several prevalent and burdensome chronic conditions were identified to follow this pattern including the skin disease hidradenitis suppurativa (HS). The first monoclonal antibody therapies developed to treat Type 3 diseases were antibodies specifically inhibiting IL-17A based on the traditional concept that this mediator carries the main pro-inflammatory activity.

The current concept behind IL-17 driven inflammation in human disease is based on the finding that two members of the IL-17 cytokine family, IL-17A and IL-17F, form dimers that exert pro-inflammatory functions after binding to IL-17 receptors composed of two receptor chains, IL-17RA and IL-17RC. Traditionally, the inflammatory potential and affinity to the RA/RC receptor was thought to decrease from IL-17A/A to IL-17A/F and to IL-17F/F, but the amount of dimers present in a given disease condition (e.g., more IL-17F/F than IL-17A/A) or the presence of different receptor types on relevant target cells (e.g. RC/RC receptors) that favor IL-17F biological activity point to an important independent pro-inflammatory role of IL-17F/F. In fact, cells exclusively producing IL-17F that do not seem to be governed by mechanisms controlling IL-17A producing cells such as stimulation by IL-23 have been identified. The IL-17F/F dimer is not inhibited by traditional IL-17A blocking monoclonal antibody therapies.

HS is a chronic inflammatory skin disease characterized by painful inflammatory lesions which may, if not adequately treated, progress to irreversible tissue destruction including formation of tunnels and scars (Sabat et al., Nat Rev Dis Primers 6, 18 (2020); Navrazhina et al. J Allergy Clin Immunol 147, 2213-2224 (2021)). The disease comes with a major impact on the quality of life and work productivity (Merchant et al., Clin Exp Dermatol. 9:llae120 (2024)). Currently there is a significant latency between the onset of first HS signs and the diagnosis and initiation of appropriate treatment (Saunte et al., Br J Dermatol 173, 1546-1549 (2015); Garg et al., J Am Acad Dermatol 82, 366-376 (2020); Tsentemeidou et al., Australas J Dermatol (2024)). Due to the lengthy time period between the first onset of HS signs and the establishment of the diagnosis and the initiation of appropriate therapies, the window of opportunity for successful medical treatment is missed in many patients. In late-stage disease when irreversible skin damage has occurred only surgical procedures remain as an option. These often involve extensive operations with wide excisions and removal of affected tissue, leading to increased morbidity and limitations for patients. It is, therefore, mandatory to identify patients with high inflammatory activity of HS and initiate optimal anti-inflammatory treatment early to prevent irreversible tissue damage.

The main clinical inflammatory phenotypes used to assess disease severity and determine appropriate HS treatments are (i) nodules, which are more superficial inflammatory lesions originating from inflamed hair follicles, (ii) abscesses, correlating to deep dermal lesions developing subsequent to hair follicle rupture and more pronounced influx of inflammatory cells, and (iii) tunnels. These tunnels are deep dermal structures that may connect and originate from ruptured follicles, but can also reach the skin surface, appearing as openings. These tunnels typically undergo re-epithelialization and become the primary source of inflammation and the driving force of HS (Active tunnels fill with neutrophils and bacteria and often ooze or ‘drain’ a malodorant pus to the skin surface. Tissue destruction subsequent to nodules, abscesses, and inflammation in and around tunnels results in scarring. When patients reach the stage of extensive scarring and tunnel formation, they often require extensive surgical intervention which can further adversely affect overall morbidity and well-being.

In contrast to other, more superficial chronic inflammatory skin disease such as psoriasis or atopic dermatitis, the presence and severity of HS lesions can hardly be assessed by clinical inspection and investigation alone, especially in the presence of tunnels. This is a major, currently unsolved problem regarding timely diagnosing HS, adequately assessing inflammatory disease activity, selecting and initiating appropriate therapy, and successfully monitoring treatment response and managing long-term therapy of HS. And although IL-17A and IL-17F have been identified as main mediators driving HS disease activity, in particular the activation of keratinocytes and the release of chemo-attractive cytokines responsible for the influx of neutrophils, T cells and other immune cells that initiate, enhance and perpetuate the inflammatory facets of HS (Lima et al., Br J Dermatol. 174(3):514-21 (2016)), there remains a need to identify biomarkers that allow for improved management of HS and other IL-17-dependent conditions. There also remains a need for early diagnosis, prediction of treatment response to existing therapies and/or analogs thereof, and the surveillance of long-term disease control. There also remains a need for more customized and effective treatment of inflammatory skin conditions. In the case of diseases such as HS or psoriatic arthritis with difficult-to-reach sites of inflammation, delivery of an IL-17F and/or IL-17A inhibiting mechanism of action would benefit from the development of targeted therapies with enhanced tissue penetration.

### BRIEF SUMMARY OF THE INVENTION

Disclosed herein are methods of treating an IL-17-dependent condition, comprising administering a medicament comprising an IL-17A- and/or IL-17F-inhibiting nanobody to a subject wherein the subject has been identified as having an elevated level of one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

Also disclosed herein are methods of treating hidradenitis suppurativa (HS), comprising administering a medicament comprising an IL-17A- and/or IL-17F-inhibitor to a subject, wherein the subject has been identified as having an elevated level of one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13. In embodiments, such a method is contemplated, wherein the IL-17A- and/or IL-17F-inhibitor comprises a nanobody.

Methods of treatment as above are also disclosed, wherein the nanobody is configured to specifically bind to IL-17A and IL-17F, preferably the nanobody is sonelokimab (SLK).

Methods of treatment as above are also disclosed, wherein the elevated level is as compared to: (i) the level present in non-lesional skin, preferably in perilesional skin, (ii) the level present in peripheral blood of a healthy patient, or (iii) the level in the same subject prior to initial treatment.

Methods of treatment as above are also disclosed, wherein the elevated level is as compared to a reference value and the reference value is (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue, preferably healthy tissue from the same subject, more preferably perilesional skin from the same subject.

Methods of treatment as above are also disclosed, which comprise assaying a tissue sample or body fluid, preferably a skin or peripheral blood sample, from a patient having or at risk of having an IL-17-dependent condition for a level of one or more of the biomarkers prior to treating.

Methods of treatment as above are also disclosed, wherein the elevated level of the one or more biomarkers indicates that the patient has (i) an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa (HS), such as moderate to severe HS, and/or (ii) a phenotype that includes one or more draining tunnels in the skin.

Methods of treatment as above are also disclosed, wherein the more elevated the level of the one or more biomarkers, the higher the number of draining tunnels are present in the subject.

Methods of treatment as above are also disclosed, wherein the subject has been clinically diagnosed as having HS in Hurley Stage I or II or III.

Methods of treatment as above are also disclosed, wherein the subject has been clinically diagnosed as having mild HS, moderate HS, moderate-to-severe HS, severe HS, or juvenile HS.

Methods of treatment as above are also disclosed, wherein the subject has no draining tunnels or wherein the subject has at least one draining tunnel.

Methods of treatment as above are also disclosed, wherein the biomarkers comprise protein and/or mRNA biomarkers.

Methods of treatment as above are also disclosed, wherein the one or more biomarkers are selected from IL6, PLA2G2A, PI3, CST7, IL17A, IL17F, GH1, MZB1, IL1B, IFNG, TN C, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

Methods of treatment as above are also disclosed, wherein the one or more biomarkers are selected from IL6, PLA2G2A, IL19, PI3, CST7, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, PDFGA, CXCR2, CCR6, and CXCL13.

Methods of treatment as above are also disclosed, wherein the one or more biomarkers are selected from CSF3, CCL2, CCL3, CCL4, CCL5, CCL7, CCL20, CXCL1, CXCL8, and PDFGA.

Methods of treatment as above are also disclosed, wherein the one or more biomarkers are selected from CCL20, CXCL8, CXCL1, IL17A, and IL17F.

Also disclosed herein is use of an agent that selectively binds to IL-17A and/or IL-17F in a subject determined to have an elevated level of at least one of the following biomarkers: IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13 compared to a healthy control, preferably wherein the elevated level is present in the skin and/or in the blood, for the use according to the present invention and any embodiment, any combination, and/or any group of biomarkers disclosed apply accordingly.

A use as above is also disclosed, wherein the elevated level of the at least one biomarker is the level present in a biological sample from the subject, and the level of the corresponding at least biomarker present in the healthy control is representative of the level present in a biological sample not affected by an IL-17-dependent condition, preferably by an IL-17-dependent inflammatory skin disease, more preferably by hidradenitis suppurativa (HS).

A use as above is also disclosed, wherein the biomarkers are associated with an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa (HS).

A use as above is also disclosed, which is characterized by an elevated mRNA level or elevated protein level of at least one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13, and preferably from at least one or more biomarkers selected from CCL20, CXCL8, CXCL1, IL17A, and IL17F.

A use as above is also disclosed, which is characterized by an elevated mRNA level or elevated protein level in a lesional skin sample compared to the respective mRNA or protein level of a non-lesional skin sample.

A use as above is also disclosed, wherein a lesional IL-17F/non-lesional IL-17 mRNA ratio or protein ratio is determined.

A use as above is also disclosed, wherein the subject has been identified as having an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa, respectively exhibiting an elevated level of one or more biomarkers.

A use as above is also disclosed, wherein the subject has been clinically diagnosed as having HS in Hurley Stage I or II or III.

A use as above is also disclosed, wherein the release of IL-17A and/or IL-17F in Hurley Stage I, II and/or III of hidradenitis suppurativa is inhibited by the agent.

A use as above is also disclosed, wherein the agent is an IL-17A- and/or IL-17F-inhibitor.

A use as above is also disclosed, wherein inflammation and/or tissue destruction is inhibited.

A use as above is also disclosed, wherein the agent comprises an antibody, an antibody fragment, or a nanobody. In embodiments of such a use, the agent may comprise a nanobody, preferably the nanobody is sonelokimab or a derivative thereof.

A use as above is also disclosed, wherein the agent is a nanobody, preferably sonelokimab or a derivative thereof, and a biomarker panel comprises a) CCL20, CXCL1, CXCL8, CXL1, IL17A, IL17F, IL19, LT01, CSF3, OSM, PLA2G2A, CST7 and/or PI3, b) IL17A, IL17F, CCL20, CXCL1, and/or CXCL8, c) IL17A, IL17F, CCL20, and/or CXCL8, d) LT01, IL17A, CSF3, OSM, PLA2G2A and/or CST7, e) CCL20, CXCL8, IL19, and PI3; or f) PI3 and IL19.

Also disclosed herein are methods of treating an IL-17-dependent condition, comprising administering a medicament comprising an IL-17A- and/or IL-17F-inhibitor to a subject wherein the subject has been identified as a having a reduced level of one or more biomarkers selected from PCDH1, BOC, MEPE, ADAM23, THOP1, IL1RL2, RCOR1, and EDAR.

Methods of treating subjects identified as having a reduced level of one or more biomarkers as above are also disclosed, wherein the IL-17A- and/or IL-17F-inhibitor comprises a nanobody.

Methods of treating subjects identified as having a reduced level of one or more biomarkers as above are also disclosed, wherein the nanobody is configured to specifically bind to IL-17A and IL-17F, preferably the nanobody is sonelokimab (SLK).

Methods of treating subjects identified as having a reduced level of one or more biomarkers as above are also disclosed, wherein the reduced level is as compared to: (i) the level present in non-lesional skin, preferably in perilesional skin, (ii) the level present in peripheral blood of a healthy patient, or (iii) the level in the same subject prior to initial treatment.

Methods of treating subjects identified as having a reduced level of one or more biomarkers as above are also disclosed, wherein the reduced level is as compared to a reference value and the reference value is (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue, preferably healthy tissue from the same subject, more preferably perilesional skin from the same subject.

Methods of treating subjects identified as having a reduced level of one or more biomarkers as above are also disclosed, which comprise assaying a tissue sample or body fluid, preferably a skin or peripheral blood sample, from a patient having or at risk of having an IL-17-dependent condition for a level of one or more of the biomarkers prior to treating.

Methods of treating subjects identified as having a reduced level of one or more biomarkers as above are also disclosed, wherein the reduced level of the one or more biomarkers indicates that the patient has (i) an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa (HS), such as moderate to severe HS, and/or (ii) a phenotype that includes one or more draining tunnels in the skin.

Methods of treating subjects identified as having a reduced level of one or more biomarkers as above are also disclosed, wherein the more reduced the level of the one or more biomarkers, the higher the number of draining tunnels are present in the subject.

Methods of treating subjects identified as having a reduced level of one or more biomarkers as above are also disclosed, wherein the subject has been clinically diagnosed as having HS in Hurley Stage I or II or III.

Methods of treating subjects identified as having a reduced level of one or more biomarkers as above are also disclosed, wherein the subject has been clinically diagnosed as having mild HS, moderate HS, moderate-to-severe HS, severe HS, or juvenile HS.

Methods of treating subjects identified as having a reduced level of one or more biomarkers as above are also disclosed, wherein the subject has no draining tunnels, or wherein the subject has at least one draining tunnel.

Methods of treating subjects identified as having a reduced level of one or more biomarkers as above are also disclosed, wherein the biomarkers comprise protein and/or mRNA biomarkers.

Also disclosed herein is use of an agent that selectively binds to IL-17A and/or IL-17F in a subject determined to have a reduced level of at least one of the following biomarkers: PCDH1, BOC, MEPE, ADAM23, THOP1, IL1RL2, RCOR1, and EDAR in the skin and/or in the blood compared to a healthy control, preferably wherein the reduced level is present in the skin and/or in the blood.

Also disclosed herein are methods of monitoring treatment progress or remission of an IL-17-dependent condition, which methods comprise assaying a biological sample from a subject previously treated for the IL-17-dependent condition, preferably hidradenitis suppurativa, for one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, CXCL13, PCDH1, BOC, MEPE, ADAM23, THOP1, IL1RL2, RCOR1, and EDAR.

Methods of monitoring as above are also disclosed, wherein the biomarkers comprise PI3 and IL19.

Methods of monitoring as above are also disclosed, wherein normalization of the level of the one or more biomarkers to the level of a healthy control indicates (i) inflammatory remission; (ii) any tunnels present in the subject remain present but are no longer active; and/or (iii) treatment is to be stopped.

Also disclosed herein are methods of identifying a subject having or at risk of having hidradenitis suppurativa (HS), which comprises determining the ratio of IL-17F/IL-17A protein or mRNA present in a tissue sample of the subject.

Also disclosed herein are methods of detecting hidradenitis suppurativa (HS), which comprises assaying a sample from a subject having or at risk of having HS for elevated levels of one or more of IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13; or for reduced levels of one or more of PCDH1, BOC, MEPE, ADAM23, THOP1, IL1RL2, RCOR1, and EDAR.

Methods of detecting as above are also disclosed, wherein the respective elevated or reduced level is as compared to: (i) the level present in non-lesional skin, preferably in perilesional skin, (ii) the level present in peripheral blood of a healthy patient, or (iii) the level in the same subject prior to an initial treatment.

Methods of detecting as above are also disclosed, wherein the respective elevated or reduced level is as compared to a reference value and the reference value is (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue, preferably healthy tissue from the same subject, more preferably perilesional skin from the same subject.

Methods of detecting as above are also disclosed, which comprise assaying a tissue sample or body fluid, preferably a skin or peripheral blood sample, from a patient having or at risk of having HS.

Also disclosed herein is a method of treating a subject having an assayed lesional IL-17F/non-lesional IL-17F protein ratio of 2 or higher, preferably 10 or higher, more preferably 2-50, and most preferably 10-30, which comprises administering an effective amount of an agent configured to inhibit IL-17F present in the dermis of an inflammatory skin lesion of the subject.

Also disclosed herein is a method of treating a subject having an assayed lesional IL-17F/non-lesional IL-17F mRNA ratio 5 or higher, preferably 10 or higher, more preferably 5-500, and most preferably 10-300, which comprises administering an effective amount of an agent configured to inhibit IL-17F present in the dermis of an inflammatory skin lesion of the subject.

Also disclosed herein is a method of treating hidradenitis suppurativa, comprising administering a medicament comprising an IL-17A- and/or IL-17F-inhibitor to a subject, wherein the subject has been identified as having elevated levels of one or more biomarkers selected from (a) LTO1, (b) CSF3, (c) OSM, (d) PLA2G2A, or (e) a combination of IL17A and TNC.

Also disclosed herein is a method of identifying a patient having hidradenitis suppurativa, which patient is responsive to treatment with an IL-17A- and/or IL-17F-inhibiting nanobody, comprising assaying a biological sample from the patient for the presence of one or more biomarkers selected from (a) LTO1, (b) CSF3, (c) OSM, (d) PLA2G2A, or (e) a combination of IL17A and TN C, wherein the presence of an elevated level of at least one, two, three, or more of the biomarkers indicates that the patient is responsive to treatment with an IL-17A- and/or IL-17F-inhibiting nanobody.

Such a method of identifying may comprise identifying a patient who is a super-responder.

Also disclosed herein is such a method of identifying, wherein the identified patient is administered treatment with an IL-17A- and/or IL-17F-inhibiting nanobody, preferably sonelokimab.

Also disclosed herein is such a method of identifying, wherein the nanobody comprises sonelokimab.

Also disclosed herein is a kit for identifying a subject at risk or in need of treatment for an IL-17-dependent condition, which kit comprises a reagent or reagents for measuring the level or expression of one or more of: IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, CXCL13, PCDH1, BOC, MEPE, ADAM23, THOP1, IL1RL2, RCOR1, and EDAR.

Such a kit as above may further comprise packaged components for obtaining a biological sample from a subject, preferably for obtaining a skin sample or biological fluid sample, optionally wherein the biological fluid sample is a blood sample.

Such kits as above may comprise an adhesive strip for obtaining a skin sample or packaged components for obtaining a punch biopsy, micro biopsy, or surgical specimen.

Also disclosed herein is a kit for treating an IL-17-dependent dermatological condition, wherein the kit comprises a pharmaceutical composition comprising an inhibitor of IL-17A and/or IL-17F, and a reagent for measuring the level or expression of IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, CXCL13, PCDH1, BOC, MEPE, ADAM23, THOP1, IL1RL2, RCOR1, and/or EDAR, in a sample taken from a subject.

### BRIEF DESCRIPTION OF THE DRAWINGS

FIGS. 1A-1D Initiation and clinical localization of Hidradenitis suppurativa (HS). Sebaceous glands (top arrow in FIG. 1A) secret into hair follicles (also termed FolliculoPiloSebaceousUnits or FPSUs). HS affects hair follicles that also contain apocrine glands (bottom arrow in FIG. 1A). Both glands connect to the hair follicle in the micro-anatomical compartment termed infrainfundibulum (area “B” in FIG. 1B). Hyperkeratinisation in this area (marked with arrows in FIG. 1B) is considered an initial event in the pathophysiology of HS leading to plugging and accumulation of gland products in the affected follicle (FIG. 1C). Consequently, HS primarily affects those body areas which harbor apocrine glands (FIG. 1D). Picture in FIG. 1B from https://plasticsurgerykey.com/the-folliculopilosebaceous-unit-the-normal-fpsu/; Accessed June 2017; von Laffert M et al. Br j Dermatol 164:367-71, 2011.

FIG. 2 Immune-mediated progression of HS. Follicular hyperkeratosis and plugging (clinical correlate: acneiform lesions) leads to changes in the local microbiome and bacterial superinfection together with initial activation of immune mechanisms (activation of antigen-presenting cells such as dendritic cells and macrophages) and influx of neutrophils (clinical correlate: folliculitis and perifolliculitis). Advanced folliculitis and rupture of the hair follicle is accompanied by enhanced inflammation (influx and activation of lymphocytes such as T cells and neutrophils) and leads to the formation of superficial nodules. A pathophysiological “vicious circle” emerges in which products released from immune cells such as IL-17A and IL-17F activate keratinocytes to release chemo-attractive mediators such as CXCL1, CXCL8 and CCL20 that further enhance immune cell influx and activation; deep inflammatory lesions (clinical correlate: abscesses) develop. Inflammatory tissue and follicle destruction leads to dermal scarring and tunnel formation. These tunnels may connect ruptured follicles deep in the dermis but may also connect to the skin surface. Likely as a consequence of stem cell activation in the outer root sheath of the bulge region of the hair follicle, these tunnels get epithelialized; the keratinocytes around tunnels will respond to activation by IL-17A and IL-17F, attracting neutrophilsto the tunnel lumen (clinical correlate: draining tunnels) and further enhancing deep dermal inflammation. Hurley stages primarily describe the absence (Stage I) or presence of a few (Stage 1l) or multiple tunnels and scars (Stage Ill). Hurley stage II and III are typically used in clinical development to define moderate to severe HS.

FIG. 3. The “vicious circle” of HS pathophysiology: main cellular players and central role of IL-17A and IL-17F in immune-mediated progression of HS. The interplay between immune cells and keratinocytes plays a crucial role in the initiation and perpetuation of HS. IL-17A and IL-17F (released from various cellular sources including CD4+ and CD8+ T cells, γδ T cells, innate lymphoid cells group 3, mucosal-associated invariant T cells (MAIT)) are the main cytokines to activate keratinocytes in HS. Activated keratinocytes proliferate (upregulation of e.g., Ki-67 and lipocalin-2) and produce chemo-attractive mediators (or chemokines) that stimulate the influx of immune cells into HS lesions. For example, chemokines stimulating the influx of immune cells are CXCL1 and CXCL8 that attract neutrophils, one of the most prominent immune cells involved in HS, and CCL20 which enhances the influx of more T cells able to produce IL-17 (Th17 cells). The chemokine-mediated influx of antigen-presenting cells (such as CD11c+ dendritic cells), T cells and neutrophils enhances cutaneous inflammation resulting in the secretion of more IL-17A and IL-17F.

FIG. 4. Clinical phenotypes of different stages of HS (which can be present simultaneously in the same patient). Early HS is typically characterized by acneiform lesions and folliculitis, while more advanced HS presents with larger more superficial (nodules) or deep inflammatory lesions (abscesses). Patients with chronic destructive HS often show various degrees of scarring and tunnel formation. Active (draining) tunnels with connection to the skin secret pus (neutrophils from the tunnel lumen) to the skin surface—one of the most burdensome aspects of HS for affected patients.

FIGS. 5A-5B. Current concept of key events in the pathophysiology of chronic HS. FIG. 5A. Immune cells are activated by antigen presenting cells such as epidermal Langerhans cells and dermal dendritic cells. Key mediators released from activated immune cells such as T cells are IL-17A and IL-17F. Both cytokines cooperatively activate keratinocytes to proliferate (upregulation of proliferation marker Ki-67 and formation of a psoriasis-like epidermal hyperplasia) and to release chemo-attractive mediators such as CXCL1 and CXCL8. IL-17A and IL-17F also activate in keratinocytes other pro-inflammatory pathways including the autocrine inflammation-enhancing secretion of IL-36 and IL-17C. CXCL1, CXCL8 and other chemokines induced in keratinocytes by IL-17A and IL-17F govern the influx of inflammatory cells such as neutrophils and T cells into HS lesions. Keratinocytes surrounding neo-epithelialized tunnels deep in the dermis represent de novo targets of IL-17A and IL-17F further enhancing dermal inflammation and the influx of neutrophils into the tunnel lumen (underlying “draining” tunnels). Image adapted from Navrazhina K, et al. J Allergy Clin Immunol 2021; 147:2213-24. FIG. 5B. Quantitative RT-PCR demonstrates higher levels of IL-17A and IL-17F mRNA in the dermal compared to the epidermal compartment in HS lesions indicating maximum inflammatory activity around dermal tunnels.

FIG. 6. Experimental approach to the identification of IL-17i-relevant HS biomarkers and the characterization of specific targeted molecule properties. Punch biopsies of specific HS phenotypes (perilesional tissue, nodule-containing tissue, tunnel-containing tissue) were obtained from larger surgical specimen of patients undergoing surgery for HS. Biopsies were first subjected to imaging [H & E staining e.g., to identify nodules and tunnels, multi-channel immunofluorescence (IF) to characterize keratinocyte activation and the immune cell infiltrate (see Work Flow at (i))]. Biopsies were then subjected to protein lysate and mRNA extraction which were further analyzed by multi-cytokine array or ELISA and bulkRNAseq and quantitative RT-RCR analyses (see Work Flow at (ii) and (iii)). Finally, selected perilesional and tunnel-containing biopsies were used for air-liquid interface organ culture to test the penetration and specific anti-inflammatory effects of IL-17-inhibiting (IL-17i) therapeutic molecules (see Work Flow at (iv)). KCs were then exposed to different IL-17 dimers to test the specific pro-inflammatory effects of IL-17A and IL-17F (see Work Flow at (v)). Different IL-17i therapeutic molecules were added to evaluate the specific inhibitory potential across different concentrations of these molecules (See Work Flow at (vi). Separately, peripheral blood from patients with HS was collected before and after treatment with an IL-17 inhibitor (IL17i), alongside collection of peripheral blood from healthy controls. Proteomics (Olink®) was used to further identify biomarkers for HS (see Work Flow at (vii)).

FIG. 7. Experimental procedureto evaluate biomarkers in the peripheral blood. Blood samples were collected from individuals with HS participating in a clinical trial (n=234), and from age and sex matched healthy controls (n=50). Clinical trial samples collected at baseline and after 12 weeks of treatment with IL-17i or placebo were analyzed using the proteomic Olink® platform, i.e., Explore 384 Inflammation & 384 Cardiometabolic panels. Greater than 700 peripheral blood proteins were investigated. NPX, Normalized Protein eX pression, is an arbitrary unit that uses a Log2 scale enabling the identification of variations in protein levels across different datasets.

FIG. 8A-8F. Validation of biopsy material used for identification of IL-17i-relevant HS biomarkers. Biopsies were investigated by hematoxylin & eosin (H&E) and multi-channel immunofluorescence staining. Microscopic analysis confirmed the presence of specific HS phenotypes (nodules and/or tunnels) in the obtained biopsies (FIG. 8A and FIG. 8B). HS-typical activation of keratinocytes (compare FIGS. 3 and 5A) was confirmed by upregulation of epithelial Ki-67 and lipocalin-2 expression (FIG. 8C, above HS nodule). Tunnel activation (draining) was confirmed by H&E and demonstration of M P0+ neutrophils in the tunnel lumen (FIG. 8B). Typical immune cell influx (e.g., CD3+ T cells, CD11c+ dendritic cells; compare FIGS. 3 and 5A) was documented (FIG. 8D shows perilesional tissue and FIG. 8E an active nodule) as well as presence of IL-17A and/or IL-17F containing T cells among infiltrating immune cells (FIG. 8F).

FIGS. 9A-9V. Tissue and blood biomarkers for HS disease and tunnel activity. mRNA: FIGS. 9A-9V. Mean±SEM normalized raw counts (RNA-seq) from patient biopsies (n=4-5) in respective skin compartments. Tissue Protein: Mean±SEM of cytokine protein levels in lysates from perilesional and lesional HS punch biopsies (n=7). P-values from Kruskal-Wallis test and uncorrected Dunn's test. Peripheral Blood Protein: Scatterplot of significant proteins associated with draining tunnel count. P-values from Kruskal-Wallis test and uncorrected Dunn's test; ***P<0.001. **P<0.01. *P<0.05.

FIG. 10. Tissue and blood biomarkers for HS disease and tunnel activity. The upregulation of biomarkers in lesional tissue is not limited to the chemokines CCL20 and CXCL8, but also extends to their receptors. mRNA: Mean±SEM normalized raw counts (RNA-seq) from patient biopsies used for protein array qualifying for RNA-seq (4-5/7) in respective skin compartments. Protein: Mean±SEM of cytokine protein levels in lysates from perilesional and lesional HS punch biopsies (n=7). P-values from Kruskal-Wallis test and uncorrected Dunn's test, ***P<0.001. **P<0.01. *P<0.05.

FIG. 11. The nanobody sonelokimab (SLK) displays high affinity for IL-17A/A and equally high affinity for IL-17F/F in contrast to the antibody bimekizumab that shows a higher affinity for IL-17A/A, but a lower affinity for IL-17F/F. Surface plasmon resonance assay (SPR) comparing equilibrium dissociation constant (K_{D}) between the IL-17A inhibiting antibody secukinumab, the IL-17A and IL-17F inhibiting antibody bimekizumab and the IL-17A and IL-17F inhibiting nanobody sonelokimab. K_{D} describes the concentration at which half of all binding sites are occupied (at equilibrium conditions, i.e. when association and dissociation rate constants are equal). When comparing the K_{D}s of two molecules, a lower K D indicates a higher affinity.

FIG. 12. Best fit tetrameric modelling of sonelokimab. The modelling indicates simultaneous binding of two IL-17 dimers and human serum albumin with preferential binding of IL-17F containing dimers.

FIG. 13. Sonelokimab inhibits interactions of IL-17 dimers with their respective receptors with high potency. Protein-protein interaction assay of IL-17 dimers with IL-17 receptor chains. IC50 analyses demonstrate high inhibitory potency of sonelokimab with a 100-fold difference in IC50 between sonelokimab and the IL-17A inhibiting antibody secukinumab.

FIG. 14. Sonelokimab treatment leads to higher IL-17 target binding capacity in the synovial fluid than an IL-17A and IL-17F binding antibody in a primate model of “human PsA”. Arthritis was induced in n˜50 female cynomolgus monkey and treatment arms included the IL-17A and IL-17F inhibiting nanobody sonelokimab and an IL-17A and IL-17F inhibiting mAb. The binding capacity for IL-17A (left bar chart) and for IL-17F (right bar chart) was determined in the synovial fluid of affected animals 8 weeks after immunization (collagen-induced arthritis). Nanobody and antibody treatment doses were corrected for different molecule size (2.8 mg/kg sonelokimab, 10 mg/kg IL-17A/F mAb). There was a much higher IL-17A and IL-17F binding capacity in the synovial fluid of the nanobody treated animals indicating a preferential accumulation in inflamed joints versus the antibody. This difference was associated with a better clinical response (upper right panel). Assessed joints for the determination of Arthritis Score. The scored joints are indicated (circles) for the large joints (top panel), for limb joints (middle panel) and hind limb joints (bottom panel). DIP, distal interphalangeal joint; PIP, proximal interphalangeal joint: MCP, Metacarpophalangeal joint: MTP, Metatarsophalangeal joint; 2 Exp IL-17A & IL-17F mAb (Novimmune); SLK=sonelokimab.

FIGS. 15A-15B. Direct evidence of disease modification with an IL-17i. FIG. 15A. Following a 12-week regimen of SLK, a significant number of patients achieved total resolution of draining tunnels (DT100), in contrast to those who were given a placebo. The percentage of patients achieving complete resolution of draining tunnels reached 49%, after 24 weeks of treatment. FIG. 15B. Ultrasound visuals revealed a substantial modification in the size and shape of the draining tunnels (indicated by dashed straight lines) in a patient undergoing IL-17i treatment. The dotted circles underscore the increased inflammatory blood flow, a defining feature of the tissue around HS tunnels. Following a 24-week IL-17i treatment period, there was a notable decrease in inflammation around draining tunnels, as evidenced by the reduced size of the dotted circles.

FIG. 16. IL-17F is highly upregulated in HS lesional compared with perilesional tissue and more abundant than IL-17A. Levels of IL-17A and IL-17F were measured by multi-cytokine array in biopsies from perilesional HS and defined HS lesions (nodules, tunnels). Compared to psoriasis (protein levels measured with a different technique in interstitial dermal fluid; ref. 1), IL-17F was more upregulated in lesional HS versus perilesional HS than in lesional versus nonlesional psoriasis. Within HS lesions (nodules and tunnels), IL-17F was more abundant than IL-17A with observed protein ratios of approximately 1.5 to 2.2. Mean±SEM of cytokine protein expression levels in lysates from perilesional (PL) and lesional HS punch biopsies (n=7 independent patients with two technical replicate measurements). Differences in IL-17F protein levels were significantly different between perilesional and nodules and tunnels, respectively (Kruskal-Wallis test and uncorrected Dunn's test, p<0.05). Ref 1=Data from Kolbinger et al. J Allergy Clin Immunol 2017:139:923-932; differences between lesional and non-lesional (NL) PsO were not significant (p>0.05).

FIG. 17. IL-17F potently activates human keratinocytes, independently of IL-17A, to release inflammatory mediators that are upregulated in HS. IL-17F stimulates production of CCL20, a major chemoattractant for Th17 cells, and CXCL8, a key chemokine for neutrophils, in keratinocytes. mRNA data are shown as mean±SEM from n=3 independent experiments following stimulation for 6 hr. CCL20 and CXCL8 gene expression is given as fold change vs. TNF (set at “1”) after normalization to GAP D H.

FIG. 18. SLK has an enhanced inhibitory effect on keratinocytes activated by IL-17 dimers. Mean±SEM percent inhibition of CCL20 and CXCL8 gene expression induced by IL-17 dimer and TNF stimulation. n=3 independent experiments; primary normal human keratinocytes were treated in vitro for 6 hr with selected combinations of TN F plus IL-17A/A, IL-17A/F or IL17F/F dimers in combination with varying concentrations of an IL-17i. qRT PCR conducted with GAPDH as the reference housekeeping gene. Mean from n=3 independent experiments. SLK, sonelokimab.

FIG. 19. Experimental approach to investigate specific inhibitory effects of an IL-17i on IL-17 relevant biomarker expression in HS organ cultures. To investigate further the inhibitory effects observed with sonelokimab on the expression of IL-17 induced chemokines in human keratinocytes, perilesional biopsies (control) and biopsies from defined HS phenotypes (nodules, tunnels) were cultured for 24 hours under air-liquid interface conditions in the presence or absence of sonelokimab. Material for the quantitative analysis of tissue chemokine mRNA expression and protein levels in the culture supernatant were obtained.

FIG. 20. Proof that an IL-17-inhibiting nanobody inhibits the release of IL-17i-relevant biomarkers in organ cultures of lesional HS. The figure illustrates the expression levels of CCL20 mRNA (left panel) and CXCL8 mRNA (right panel) in perilesional skin sample. It further highlights the increased expression of CCL20 and CXCL8 in HS lesions containing tunnels, and how their expression is reduced to levels comparable to those found in perilesional HS samples when treated with IL-17i sonelokimab. CCL20 mRNA and CXCL8 mRNA relative expression levels (mean±SEM) were measured by qRT-PCR in air-liquid interface cultures of perilesional and tunnel biopsies from n=4 independent donors following 24-hr treatment ex vivo with SLK 10 μg/mL or vehicle buffer (VEH). Mean expression of the HECT, UBA And WWE Domain Containing E3 Ubiquitin Protein Ligase 1 (HUWE11), and Microtubule Actin Crosslinking Factor 1 (MACF1) was used as housekeeping internal reference.

FIG. 21. Pharmacodynamic biomarkers: After treatment with an IL-17i, the levels of circulating biomarkers related to HS activity were normalized to those found in a healthy control population indicating molecular healing (n=50). Significant downregulation of Elafin (left) and IL-19 (right) from Baseline to Week 12 following sonelokimab treatment. HC, healthy controls; SLK, sonelokimab; ADA, adalimumab. Paired t-test, ***P<0.001. **P<0.01. *P<0.05.

FIG. 22. Biomarkers to identify HS super-responders to IL-17i: LOT1, IL-17A+TNC, G-CSF, OSM, PLA2G2A. The ‘Subgroup Identification based on Differential Effect Search’ (SIDES) analysis was employed as a partitioning method to determine treatment responses within specific patient groups. Subgroups of patients with higher biomarker expression levels demonstrated an enhanced clinical outcome, as evidenced by the delta to placebo in HiSCR75 response compared to unselected patients. Applied threshold (NPX): LTO1>−0.4123; IL-17A>−0.8321; TNC>−0.0153; CSF3>−0.2455; OSM>−0.4974; PLA2G2A>0.6373; CST7>−0.628.

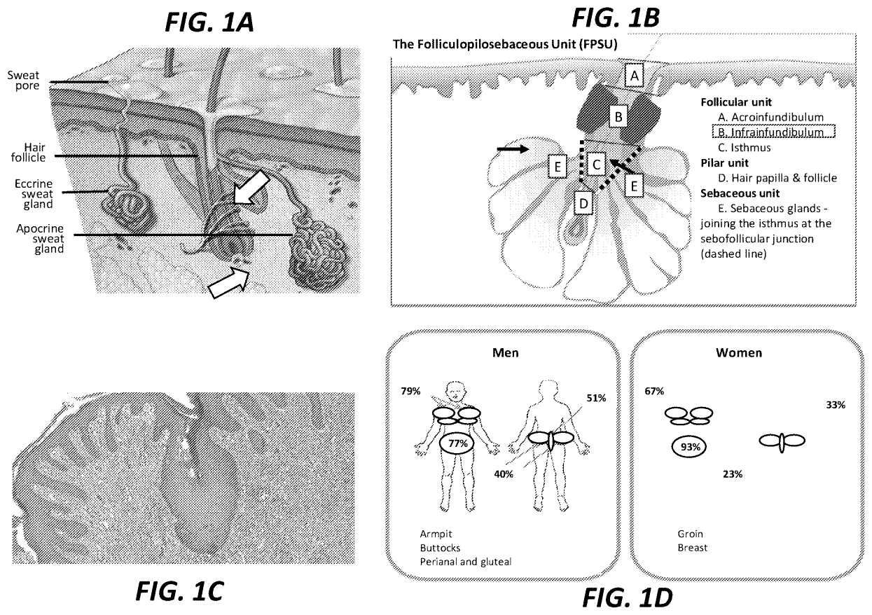

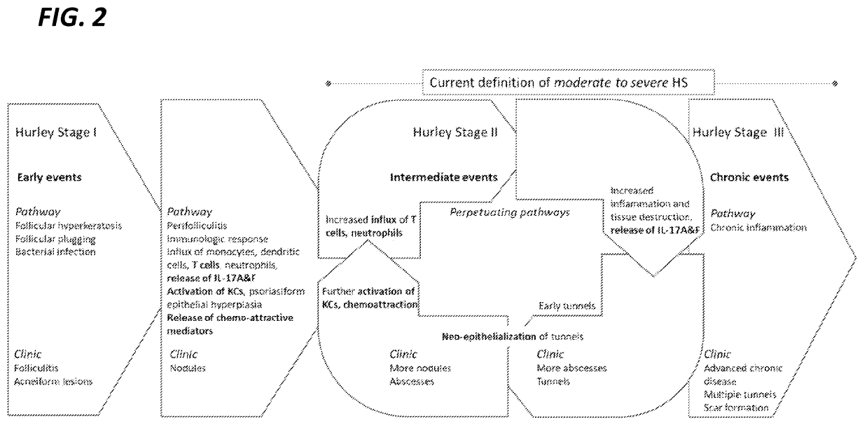

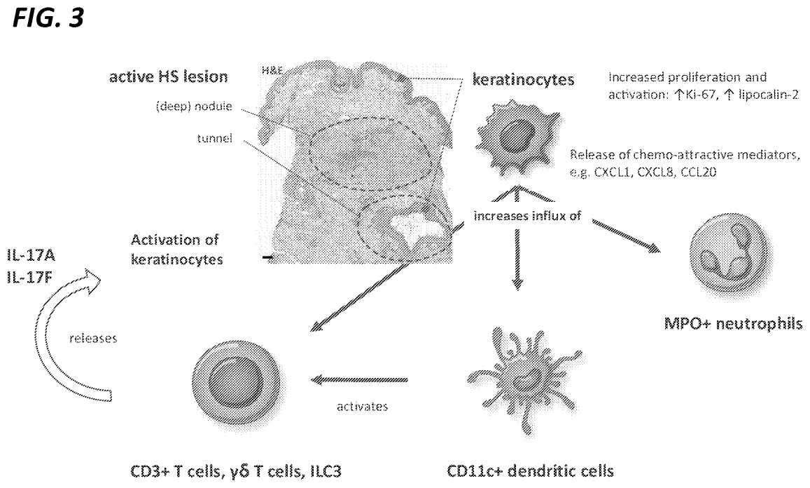

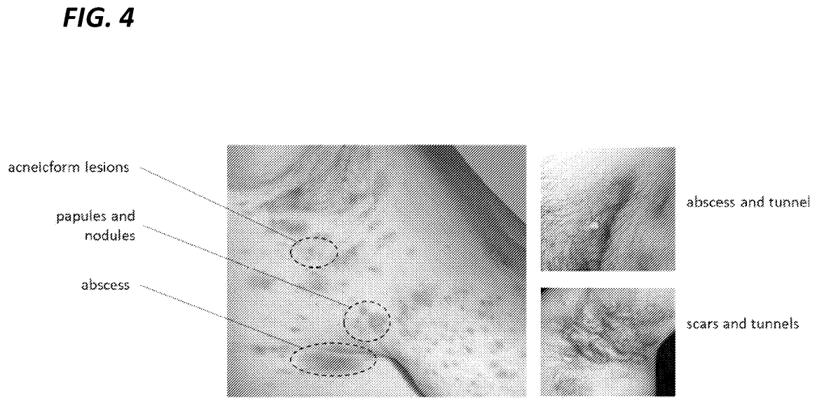

### DETAILED DESCRIPTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Applicant also contemplates ranges derived from data points and express ranges disclosed herein.

### Definitions

As used herein “treatment” and/or “treating” and/or “treat” refer to all methods or regimens wherein there may be slowing, interrupting, arresting, stopping, preventing, or reversion of the progression of the disorders described herein. However, these terms do not necessarily indicate a total elimination of all symptoms of the disorder subject to the treatment. Treatment includes administration of medicament for treatment of a disease or condition in a subject that would, for example, benefit from a reduction in activity of an elevated biomarker.

Accordingly, treatment may include inhibiting further progression of the disease, i.e., arresting its development and/or relieving the disease, i.e., causing regression of the disease or disorder or alleviating symptoms or complications thereof.

As used herein an “IL-17-dependent condition” refers to a diseases, disorder, or condition mediated by IL-17 and may include autoimmune, inflammatory, or neurologic diseases. IL-17-dependent conditions as contemplated herein preferentially include skin diseases, but are not limited to skin diseases and include, for example, psoriasis, psoriatic arthritis, rheumatoid arthritis, and inflammatory skin diseases, including Type Ill non-communicable inflammatory skin diseases. Type III diseases are defined by Th17 immunity and the presence of neutrophils in the skin and include psoriasis, including pustular psoriasis, guttate psoriasis, pityriasis rubra pilaris, acne and acne syndromes, hidradenitis suppurativa, and folliculitis decalvans.

As used herein “draining tunnel” or “active tunnel” refers to a symptom of moderate to severe HS in which epithelialized tissue forms one or more tunnel-like openings in the dermis optionally having connection to the skin surface and wherein the opening is actively draining, i.e., containing pus that may be discharged to the skin surface.

A “healthy control” as used herein may refer to the determined level of one or more biomarkers in a non-affected biological sample comprising the one or more biomarkers. The biological sample may be isolated from the same subject suffering from the disease within the meaning of the present invention but from a body part without any signs or symptoms associated with the disease. Alternatively, the biological sample may be taken from another healthy subject from a corresponding body part wherein the patient suffering from the disease exhibits the characterized symptoms. The method for tissue collection, blood samples, RNA extraction, and protein isolation are well known to the skilled person. Examples of suitable methods are shown herein.

A “derivative” of a polypeptide (e.g., nanobody) as disclosed herein may include one or more amino acid substitutions, additions, insertions, or deletions compared to the reference polypeptide, for example, 1, 2, 3, 4, 5, 6-20, or 21-50 amino acid substitutions, additions, insertions, or deletions. Alternatively, the “derivative” may have a certain percentage of sequence identity with the reference polypeptide and/or include amino acid substitutions, additions, insertions, or deletions as otherwise described herein.

### Biomarkers, Combinations of Biomarkers, and Corresponding Methods

Herein we describe the identification of biomarkers with aberrant expression in the peripheral blood and/or HS lesions that allow the assessment of immunological disease activity and especially HS and active tunnels in HS patients. We also describe how the biomarkers may be utilized to monitor the molecular immunological response to IL-17 inhibiting drugs and identify patients with preferential response (so called super-responders) to IL-17 inhibiting drugs such as sonelokimab. Biomarkers as contemplated herein include “analytes” determined by methods as described herein and by methods of the invention. A biomarker as contemplated and disclosed herein can, however, be used as such, i.e., as a functional biomarker for the diseases and/or conditions described herein.

Whether one or more of the biomarkers or whether a panel/combination of biomarkers is most appropriate within the meaning of the invention may depend on the subject, the stage of the disease, and individual phenotype. W e herein provide suitable biomarkers within the meaning of the invention from which any combination of one, two, three, four, five, six, seven, eight, nine, ten, or more biomarkers may be utilized.

Notably, expression of the following biomarkers was observed to be elevated in HS: IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

In contrast, expression of the following biomarkers was observed to be reduced in HS: PCDH1, BOC, MEPE, ADAM23, THOP1, IL1RL2, RCOR1, and EDAR.

Biomarkers for use herein, may include any selected from Table 1 or homologous wild type proteins to those disclosed in Table 1. Isoforms and variants thereof are also contemplated.

For example, a sequence homologous to the amino acid sequence represented by SEQ ID NO: 1 may function as a biomarker for an IL-17-dependent condition as described herein, and may be a protein having the same amino acid sequence as the amino acid sequence represented by SEQ ID NO: 1 except that one or several amino acids are deleted, substituted, inserted, and/or added. In the case of substitution, insertion, or addition, conservative mutations resulting from conservative substitution, insertion, or addition of one or several amino acids are possible. “One or several amino acids” herein means 1 to 50, preferably 1 to 20, more preferably 1 to 10, still more preferably 1 to 5, or 1 to 3 amino acids.

Moreover, by way of example a protein having an amino acid sequence homologous to the amino acid sequence represented by SEQ ID NO: 1 includes a protein having an amino acid sequence with an identity of not less than 60% to the amino acid sequence represented by SEQ ID NO: 1, in its full-length form. The protein includes a protein having an amino acid sequence with an identity of not less than 70%, preferably not less than 80%, more preferably not less than 90%, and still more preferably not less than 91%, 92%, 93%, 94%, 95%, 96%, 97%, 98%, or 99% to the above-described amino acid sequence in its full-length form. The same applies for the remaining SEQ ID NOs: 2-73, for other biomarkers as contemplated herein, and for nucleic acid sequences encoding such biomarkers or sequences.

“Sequence identity” may refer, in nucleotide sequences or amino acid sequences, to the percentage of identical nucleotides or amino acids shared between two sequences, which percentage is determined by aligning those two sequences in an optimal pairwise alignment, optionally by using a conventional or commercially available algorithm.

Disclosed herein are methods of treating an IL-17-dependent condition, comprising administering a medicament comprising an IL-17A- and/or IL-17F-inhibiting nanobody to a subject wherein the subject has been identified as having an elevated level of one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

Also disclosed herein are methods of treating hidradenitis suppurativa (HS), comprising administering a medicament comprising an IL-17A- and/or IL-17F-inhibitor to a subject, wherein the subject has been identified as having an elevated level of one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

Also contemplated herein is use of an agent that selectively binds to IL-17A and/or IL-17F in a subject determined to have an elevated level of at least one of the following biomarkers: IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13 compared to a healthy control, preferably wherein the elevated level is present in the skin and/or in the blood.

Such methods and/or uses may comprise assaying a tissue sample or body fluid, preferably a skin or peripheral blood sample, from a patient having or at risk of having an IL-17-dependent condition for a level of one or more of the biomarkers prior to treating.

Methods and/or uses as the above are contemplated, wherein the elevated level of the one or more biomarkers indicates that the patient has (i) an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa (HS), such as moderate to severe HS, and/or (ii) a phenotype that includes one or more draining tunnels in the skin.

In embodiments, the more elevated the level of the one or more biomarkers, the higher the number of draining tunnels are present in the subject.

In embodiments, the subject may have been clinically diagnosed as having HS in Hurley Stage I or II or III.

In embodiments, the subject may have been clinically diagnosed as having mild HS, moderate HS, moderate-to-severe HS, severe HS, or juvenile HS.

In an embodiment, the subject may have no draining tunnels or, alternatively, at least one draining tunnel.

The biomarkers as disclosed herein may consist of or comprise protein. Alternatively, the biomarkers may consist of or comprise mRNA. Both protein and mRNA biomarkers are contemplated.

As contemplated herein, the elevated level of the at least one biomarker may be the level present in a biological sample from the subject, and the level of the corresponding at least one biomarker present in the healthy control may be representative of the level present in a biological sample not affected by an IL-17-dependent condition, preferably by an IL-17-dependent inflammatory skin disease, more preferably by HS.

As contemplated herein, the biomarkers may be associated with an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa (HS).

In a preferred embodiment, the one or more biomarkers may be selected from the following combination of biomarkers: IL6, PLA2G2A, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

In another preferred embodiment, the one or more biomarkers may be selected from the following combination of biomarkers: IL6, PLA2G2A, IL19, PI3, CST7, GH1, MZB1, I1L11B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, PDFGA, CXCR2, CCR6, and CXCL13.

In another preferred embodiment, the one or more biomarkers may be selected from the following combination of biomarkers: CSF3, CCL2, CCL3, CCL4, CCL5, CCL7, CCL20, CXCL1, CXCL8, and PDFGA.

Methods and uses as above may also be characterized by an elevated mRNA level or elevated protein level of at least one or more biomarkers selected from the following combination of biomarkers: CCL20, CXCL8, CXCL1, IL17A, and IL17F.

In embodiments, combinations or panels of biomarkers may be formed based on physiological function of the biomarker, for example, as disclosed in Table 1. In an exemplary embodiment, a person of skill in the art may determine that a panel of biomarkers associated with neutrophil activation orT cell activation may be advantageous. Substitution of one neutrophil activating biomarker from Table 1 for another from Table 1 in any give combination is also contemplated.

Methods and uses as above may be characterized by an elevated mRNA level or elevated protein level in a lesional skin sample compared to the respective mRNA or protein level of a non-lesional skin sample.

Methods and uses as above are also contemplated, wherein the subject has been identified as having an inflammatory skin disease, preferably hidradenitis suppurativa, exhibiting an elevated level of one or more biomarkers.

Methods and uses as above are also contemplated, wherein the release of IL-17A and/or IL-17F in Hurley Stage I, 11 and/or III of hidradenitis suppurativa is inhibited by the agent.

Methods and uses as above are also contemplated, wherein the agent is an IL-17A- and/or IL-17F-inhibitor.

Methods and uses as above are also contemplated, wherein the agent comprises an antibody, an antibody fragment, or a nanobody.

In a preferred embodiment, methods and/or uses as above are contemplated wherein the agent is a nanobody, preferably sonelokimab or a derivative thereof, and a biomarker panel comprises a) CCL20, CXCL1, CXCL8, CXL1, IL17A, IL17F, IL19, LT01, CSF3, OSM, PLA2G2A, CST7 and/or PI3, b) IL17A, IL17F, CCL20, CXCL1, and/or CXCL8, c) IL17A, IL17F, CCL20, and/or CXCL8, d) LT01, IL17A+TNC, CSF3, OSM, PLA2G2A and/or CST7, e) CCL20, CXCL8, IL19, and PI3; or f) PI3 and IL19.

In an embodiment such methods and/or uses may further comprise comparing the level of expression of the at least one biomarker in a lesional skin sample of the subject with the level of expression of the at least one biomarker in the healthy skin sample of the subject, and determining that the subject has or is at risk of having HS if one or more of the biomarkers is elevated in the lesional skin sample compared to the healthy skin sample.

Also disclosed herein are methods of treating an IL-17-dependent condition, comprising administering a medicament comprising an IL-17A- and/or IL-17F-inhibitor to a subject wherein the subject has been identified as a having a reduced level of one or more biomarkers selected from PCDH1, BOC, MEPE, ADAM23, THOP1, IL1RL2, RCOR1, and EDAR.

Also contemplated herein is use of an agent that selectively binds to IL-17A and/or IL-17F in a subject determined to have a reduced level of at least one of the following biomarkers: PCDH1, BOC, MEPE, ADAM23, THOP1, IL1RL2, RCOR1, and EDA R in the skin and/or in the blood compared to a healthy control, preferably wherein the reduced level is present in the skin and/or in the blood.

As above, the IL-17A- and/or IL-17F-inhibitor may comprise an antibody, antibody fragment, or a nanobody. In embodiments, the nanobody may be configured to specifically bind to IL-17A and IL-17F, preferably the nanobody is SLK.

As above, the reduced level in the subject may be as compared to: (i) the level present in non-lesional skin, preferably in perilesional skin, (ii) the level present in peripheral blood of a healthy patient, or (iii) the level in the same subject prior to initial treatment.

Alternatively, the reduced level may be as compared to a reference value and the reference value may be (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue, preferably healthy tissue from the same subject, more preferably perilesional skin from the same subject.

In embodiments, methods as above involving the use of biomarkers with reduced expression may comprise assaying a tissue sample or body fluid, preferably a skin or peripheral blood sample, from a patient having or at risk of having an IL-17-dependent condition for a level of one or more of the biomarkers prior to treating.

In embodiments, a reduced level of the one or more biomarkers may indicate that the subject has (i) an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa (HS), such as moderate to severe HS, and/or (ii) a phenotype that includes one or more draining tunnels in the skin.

In embodiments, the more reduced the level of the one or more biomarkers, the higher the number of draining tunnels may be present in the subject.

Methods as above involving the use of biomarkers with reduced expression are also contemplated, wherein (i) the subject has been clinically diagnosed as having HS in Hurley Stage I or II or Ill; (ii) wherein the subject has been clinically diagnosed as having mild HS, moderate HS, moderate-to-severe HS, severe HS, or juvenile HS; (iii) wherein the subject has no draining tunnels; and/or (iv) wherein the subject has at least one draining tunnel.

As above, such methods and uses are contemplated wherein the biomarkers comprise protein and/or mRNA biomarkers.

Methods of treatment as contemplated herein may also include methods comprising treating a subject having an assayed lesional IL-17F/non-lesional IL-17F mRNA ratio of 5 or higher, preferably of 10 or higher, more preferably of 5-500, and most preferably of 10-300. Here such methods may comprise administering to the subject an effective amount of an agent configured to inhibit IL-17F present in the dermis of an inflammatory skin lesion of the subject.

Also contemplated herein is a method of treating hidradenitis suppurativa, comprising administering a medicament comprising an IL-17A- and/or IL-17F-inhibitor to a subject, wherein the subject has been identified as having elevated levels of one or more biomarkers selected from (a) LTO1, (b) CSF3, (c) OSM, (d) PLA2G2A, or (e) a combination of IL17A and TNC.

### Diagnostic Methods

Also contemplated herein are diagnostic methods and methods of, for example, detecting hidradenitis suppurativa (HS), which comprise assaying a sample from a subject having or at risk of having HS for elevated levels of one or more of IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13; or for reduced levels of one or more of PCDH1, BOC, MEPE, ADAM23, THOP1, IL1RL2, RCOR1, and EDAR.

In embodiments, the respective elevated or reduced level may be as compared to: (i) the level present in non-lesional skin, preferably in perilesional skin, (ii) the level present in peripheral blood of a healthy patient, or (iii) the level in the same subject prior to an initial treatment.

In alternative embodiments, the respective elevated or reduced level may be as compared to a reference value wherein the reference value is (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue, preferably healthy tissue from the same subject, more preferably perilesional skin from the same subject.

Diagnostic methods as above may comprise assaying a tissue sample or body fluid, preferably a skin or peripheral blood sample, from a patient having or at risk of having HS.

In embodiments diagnostic methods as above may involve identifying a subject having or at risk of having hidradenitis suppurativa (HS), by determining the ratio of IL-17F/IL-17A protein or mRNA present in a tissue sample of the subject.

Also contemplated herein aresuch methodswherein an assayed lesional IL-17F/non-lesional IL-17F protein ratio of 2 or higher, preferably 10 or higher, more preferably 2-50, and most preferably 10-30, indicates the subject has or is at risk of having HS.

Also contemplated herein are diagnostic methods, wherein an IL-17F/IL-17A protein ratio of 1.5 or higher, preferably a ratio of 1.5-3.0, most preferably a ratio 1.5-2.2, indicates that the subject has or is at risk of having HS. In an embodiment, such method may comprise obtaining a tissue sample from the subject and then measuring the ratio of IL-17F/IL-17A mRNA in the obtained tissue sample.

Methods of identifying subject responsive to treatment are also disclosed herein. For example, a method of identifying a patient having hidradenitis suppurativa is contemplated, which patient is responsive to treatment with an IL-17A- and/or IL-17F-inhibiting nanobody, the method comprising assaying a biological sample from the patient for the presence of one or more biomarkers selected from (a) LTO1, (b) CSF3, (c) OSM, (d) PLA2G2A, or (e) a combination of IL17A and TNC. Presence of an elevated level of at least one, two, three, or more of the biomarkers indicates that the patient is highly responsive to treatment with an IL-17A- and/or IL-17F-inhibiting nanobody (so-called super-responders).

In an embodiment of the above methods of identifying, the nanobody may comprise SLK.

Also disclosed herein are methods of identifying a subject having or at risk of having hidradenitis suppurativa (HS), which comprise assaying for the level of expression of at least one biomarker selected from CCL2, CCL3, CCL4, CCL5, CCL7, CCL20, CXCL1, CXCL8, PDFGAA, and CSF3 in a tissue sample of the subject.

In an embodiment, such methods may comprise assaying for the level of expression of at least one biomarker selected from CCL20, CXCL1, and/or CXCL8, in a tissue sample of the subject.

In another aspect, biomarkers CCL20, CXCL8, CXCL1, IL17A and/or IL17F may be sufficient and effective to identify a patient having HS or highly responsive to an agent for treating HS. The skilled person may consider use of a biomarker panel comprising IL19, PI3, IL17A, IL17F, CCL7 and/or CCL3, a biomarker panel comprising LT01, IL17A, CSF3, OSM, PLA2G2A and/or CST7, or any combination of the groups or different combination of the biomarkers of the aforementioned groups for this purpose. In another embodiment the combination of IL19 and PI3 may be sufficient. The skilled person is free to combine any combination of biomarkers from Table 1 within the meaning of the invention.

In another embodiment it is appropriate and advantageous to use a panel of biomarkers of at least CCL20, CXCL8, CXL1, 1L17A and/or IL17F for diagnosis and another biomarker panel for the monitoring of the treatment over time. For the skilled person it is clear that biomarkers representative of the course of pathogenesis are not necessarily the same as for diagnosing a subject at risk of having HS or of identifying a super-responder.

In an embodiment the level of expression of at least CCL20 and CXCL8 may be assessed for diagnosis of HS. In another embodiment a combination of CCL20, CXCL8, and PI3 may be used for this purpose.

Also disclosed herein is a method of predicting efficacy of a medicament in a subject having an IL-17-dependent dermatological condition, which method comprises: determining the level or expression of IL-17F protein in lesional skin sample from the subject and the level or expression of IL-17F protein in a non-lesional skin sample from the subject, wherein the medicament comprises an agent that inhibits IL-17F; wherein a lesional IL-17F/non-lesional IL-17F protein ratio of 2 or higher, preferably 10 or higher, more preferably 2-50, and most preferably 10-30, indicates that the medicament will be effective in inhibiting or treating the IL-17-dependent dermatological condition in the subject; and wherein the subject is a human or a non-human animal.

Also disclosed herein is a method of predicting efficacy of a medicament in a subject having an IL-17-dependent dermatological condition, which method comprises determining the level or expression of IL-17F mRNA in lesional skin sample from the subject and the level or expression of IL-17F mRNA in a non-lesional skin sample from the subject, wherein the medicament comprises an agent that inhibits IL-17F; wherein a lesional IL-17F/non-lesional IL-17 mRNA ratio of 5 or higher, preferably 10 or higher, more preferably 5-500, and most preferably 10-300, indicates that the medicament will be effective in inhibiting or treating the IL-17-dependent dermatological condition in the subject; and wherein the subject is a human or a non-human animal.

In an embodiment, contemplated herein is such a method, wherein the IL-17-dependent dermatological condition is hidradenitis suppurativa.

In an embodiment, the method may further comprise administering the medicament to the subject.

Also disclosed herein is a method of predicting efficacy of a medicament in a subject having an IL-17-dependent dermatological condition, which method comprises determining the level or expression of IL-17F and IL-17A present in a tissue sample of the subject prior to an initial treatment period with the medicament; wherein the medicament comprises an agent that inhibits IL-17F; wherein an IL-17F/IL-17A ratio of 1.5 or higher, preferably a ratio of 1.5-3, most preferably a ratio of 1.5-2.2, in the tissue sample indicates that the medicament will be effective in inhibiting or treating the IL-17-dependent dermatological condition in the subject, and wherein the subject is a human or a non-human animal.

In an embodiment, the IL-17-dependent dermatological condition may be hidradenitis suppurativa.

In an embodiment, such a method may further comprise administering the medicament to the subject.

In an embodiment, contemplated herein is such a method, wherein the initial treatment period is at least one week, at least two weeks, at least four weeks, at least eight weeks, at least twelve weeks, at least eighteen weeks, or at least twenty-four weeks.

### Monitoring Progress, Remission, and Therapeutic Need of an IL-17-Dependent Condition

Also contemplated herein are methods of monitoring treatment progress or remission of an IL-17-dependent condition, comprising assaying a biological sample from a subject previously treated for the IL-17-dependent condition, preferably hidradenitis suppurativa, for one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, CXCL13, PCDH1, BOC, MEPE, ADAM23, THOP1, IL1RL2, RCOR1, and EDAR.

In an embodiment, the biomarkers may comprise PI3 and IL19.

Methods monitoring treatment progress or remission as above are also contemplated, wherein normalization of the level of the one or more biomarkers to the level of a healthy control indicates (i) inflammatory remission; (ii) any tunnels present in the subject remain present but are no longer active; and/or (iii) treatment is to be stopped.

As disclosed herein such methods may preferably comprise monitoring treatment progress or remission of an inflammatory skin disease, preferably an inflammatory skin disease afflicting both the epidermis and dermis, more preferably an inflammatory skin disease involving hair follicle structures, even more preferably an inflammatory skin disease involving acneiform lesions, most preferably hidradenitis suppurativa.

Also as disclosed herein such methods may comprise steps such as determining, prior to assaying a biological sample from a subject previously treated for the IL-17-dependent condition, whether an mRNA and/or protein level of one or more biomarkers is elevated in a biological sample from a subject suffering from the IL-17-dependent condition compared to a healthy level of mRNA and/or protein of the same one or biomarkers in a healthy or non-affected biological sample. Such methods may further or alternatively comprise determining the mRNA and/or protein level of one or more biomarkers in at least one other biological sample from the subject suffering from the IL-17-dependent condition, wherein the at least one other biological sample has been obtained at a time after treatment as has been initiated.

In embodiments, a subject previously treated for the IL-17-dependent condition was treated with an IL-17A- and/or IL-17F-inhibiting nanobody, preferably sonelokimab or a derivative thereof is used.

In embodiments, such methods as above are contemplated in which the mRNA and/or protein level of one or more biomarkers in the at least one other biological sample is determined after one day, two days, three days or more days, one week, two weeks or more weeks, after 12 weeks, respectively after initial treatment.

Also contemplated herein are methods which comprise: administering an IL-17 inhibitor for a total of at least 4 weeks and preferably for a total of no more than 24 weeks to a subjected identified by one or more of the biomarkers disclosed herein, pausing the treatment for a period of more than two weeks, and then re-initiating treatment if one or more symptoms of the IL-17-dependent condition recur or if one or more of the biomarkers expression becomes aberrant again. In an embodiment, administration may be re-started when the subject has Hidradenitis Suppurativa Clinical Response (HiSCR) score below a certain threshold.

Alternatively, administration may be re-started when the subject has an International Hidradenitis Suppurativa Severity Score (IHS4) below a certain threshold. In an embodiment, re-initiating treatment may comprise administering an effective amount of the inhibitor with either a new induction dosing scheme or re-initiating the maintenance dosing schedule.

In an embodiment, the subject responsive to treatment may have an HiSCR score of 75 to 90, preferably an HiSCR score of 90 or higher (e.g. 90-100 or 91-100). Alternatively, in an embodiment the subject responsive to treatment may have an IHS4 score of 75 to 90, preferably an IHS4 score of 90 or higher (e.g. 90-100 or 91-100). Such a score may be achieved after a period of 4 weeks or more of treatment, after a period of 8 weeks or more of treatment, after a period of 10 weeks or more of treatment, after a period of 12 weeks or more of treatment, after a period of 14 weeks or more of treatment, after a period of 16 weeks or more of treatment, after a period of 18 weeks or more of treatment, after a period of 20 weeks or more of treatment, or after a period of 24 weeks of treatment. In such an embodiment, the subject responsive to treatment has or is at risk of having hidradenitis suppurativa and may have withdrawn from prior treatment with an IL-17 inhibitor after a period of 4 weeks or more, after a period of 8 weeks or more, after a period of 10 weeks or more, after a period of 12 weeks or more, after a period of 14 weeks or more, after a period of 16 weeks or more, after a period of 18 weeks or more, after a period of 20 weeks or more, or after a period of 24 weeks.

Assessment and/or Measurement of Elevated or Reduced Levels of Biomarkers

The manner in which the biomarkers disclosed herein may be measured or assessed is not particularly limited, and may include one or more of the following (i) measurement of protein levels in the peripheral blood, (ii) measurement of mRNA from peripheral blood cells or circulating DNA, (iii) analysis of surface markers (e.g. in the case of cytokine receptors or receptors to other mediators) on peripheral cells, (iv) analysis of intracellular markers in peripheral cells by in situ techniques (e.g. flow cytometry), (v) all measurements described in (i) to (iv) in other body fluids including but not limited to CSF, breast milk, amnionic fluid, urine, saliva, serous fluid, (vi) measurement of protein levels in tissue or cells derived from tissue (vii) including tissue or cells collected by adhesives applied to the skin surface, (viii) measurement of RNA in tissue (e.g. quantitative RT-PCR or bulkRNAseq) or in cells derived from tissue (e.g. single cell RNAseq) (ix) including measurement of RNA in tissue or cells collected by adhesives applied to the skin surface, (x) measurement of changes in biomarker quantity or function induced by genetic variations including but not limited to single nucleotide polymorphisms, insertions, deletions, copy number variations, translocations and inversions and/or (xi) epigenetic modifications including but not limited to changes in histones, DNA methylation and/or non-coding RNAs.

The level or amount of expression of the one or more biomarkers may be determined in vitro, e.g., from an in vitro sample, or from an ex vivo sample. The in vitro determination may then be followed by administration of an IL-17 inhibitor as contemplated herein to the subject in vivo.

In methods and uses as contemplated herein, the elevated level may be as compared to: (i) the level present in non-lesional skin, preferably in perilesional skin, (ii) the level present in peripheral blood of a healthy patient, or (iii) the level in the same subject prior to initial treatment.

In methods and uses as contemplated herein, the elevated level may be as compared to a reference value wherein the reference value is (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue, preferably healthy tissue from the same subject, more preferably perilesional skin from the same subject.

In a preferred embodiment, the elevated level of an mRNA biomarker determined in an affected biological sample, may be compared with the mRNA level of the same biomarker determined in a non-affected biological sample (=healthy level). The biological samples may be from the same subject suffering from the disease according to the present invention but from different body regions—healthy region and diseased region—or the healthy control may be obtained from a biological sample from another subject not suffering from the disease according to the present invention. The affected biological sample may be obtained from lesional skin or blood and the non-affected biological sample from non-lesional skin or blood, preferably perilesional skin, of the same subject or from another subject.

In the present disclosure, the term “elevated” may refer to an increase, for example of a level of a particular biomarker, as compared to a reference value (e.g., healthy control). The degree of increase may be, for example, at least 5%, at least 6%, at least 7%, at least 8%, at least 9%, at least 10%, at least 15%, at least 20%, at least 25%, at least 30%, at least 35%, at least 40%, at least 45%, or at least 50%, or at least 100%, or multiples of at least 100%.

In the present disclosure, the term “reduced” may refer to a decrease, for example of a level of a particular biomarker, as compared to a reference value (e.g., healthy control). The degree of decrease may be, for example, at least 5%, at least 6%, at least 7%, at least 8%, at least 9%, at least 10%, at least 15%, at least 20%, at least 25%, at least 30%, at least 35%, at least 40%, at least 45%, or at least 50%, or at least 100%, or multiples of at least 100%.

“Elevated” or “reduced” levels of one or more biomarkers as contemplated herein also may be assessed based on fold differences in the level present in a biological sample compared to a healthy control (e.g., reference value or level present in healthy tissue or blood of the same/different subject). For example, an “elevated” level of a biomarker as contemplated herein may exhibit a fold increase of 1.1, 1.2, 1.3, 1.4, 1.5, 1.6, 1.7, 1.8, 1.9, 2.0, 2.5, 3.0, 3.5, 4.5, 5.0, 5.5, 6.0, 6.5, 7.0, 7.5, 8.0, 8.5, 9.0, 9.5, 10, 20, 30, 40, 50, or at least 50 fold, or at least 100 fold or multiples thereof compared to a healthy control. By way of another example, an “reduced” level of a biomarker as contemplated may exhibit a fold decrease of 1.1, 1.2, 1.3, 1.4, 1.5, 1.6, 1.7, 1.8, 1.9, 2.0, 2.5, 3.0, 3.5, 4.5, 5.0, 5.5, 6.0, 6.5, 7.0, 7.5, 8.0, 8.5, 9.0, 9.5, 10, 20, 30, 40, 50, or at least 50 fold, or at least 100 fold or multiples thereof compared to a healthy control.

The mRNA level and/or protein level may be determined for a biopsy sample, for a tissue sample, or for a peripheral blood sample. The mRNA level or protein level may be based on the overall level of mRNA or protein of multiple samples. Alternatively, the mRNA level or protein level may be based on one specific category of samples (e.g., peripheral blood, HS lesions, or nodules) or one individual sample. There may be advantages to monitoring both protein and mRNA levels as some biomarkers may be regulated on the transcriptional and/or translational level.

### Subjects

In the present disclosure, the term “subject,” or similar terms such as “patient” or “individual,” may refer to the recipient of the medicament or may refer to the individual from whom a biological sample has been obtained for biomarker assessment. This subject may be, for example, a mammal, preferably a human. The human is not limited, and may be of any disease state, weight, age or gender. For example, the subject may be a human adolescent, i.e., a person of age 12-17, or a juvenile, or a child of 1-11, 2-10, 3-9, or 6-12 years of age, or an infant (e.g., an infant of at least six months of age). The subject may be one which has, or is at risk of developing, a disease, disorder, or condition, which would benefit from biomarker measurement and/or treatment as disclosed herein. Additionally, the subject may be an animal, including without limitation, a non-human animal including, for example, livestock (such as cattle, sheep, pigs, goats, horses, donkeys, mules, buffalo, oxen, llamas, alpacas, or camels) or domesticated animals (such as dogs or cats).

The present disclosure identifies subjects having one or more of the biomarkers of Table 1, or any combination or combinations thereof, or having a minimum of 1, 2, 3, 4, 5, 6, 7, 8, 9, 10, 15, 20 or more thereof, as constituting a new patient group which may, for example, be treated with an IL-17 inhibitor and/or be particularly responsive to treatment with an IL-17 inhibitor and/or whose disease progress or remission may be monitored on the basis of the expression levels of the biomarker(s) present in a biological sample (e.g., vs. a healthy control or suitable reference value).

The subject may have any of the IL-17-dependent conditions disclosed herein or be at risk for having any such condition.

In embodiments, the subject may have been clinically diagnosed as having HS in Hurley Stage I or II or III.

In embodiments, the subject may have been clinically diagnosed as having mild HS, moderate HS, moderate-to-severe HS, severe HS, or juvenile HS.

### Biological Sample

A “sample” or “biological sample” as used herein refers to a sample derived from a subject. Non-limiting sources of a sample include blood, plasma, serum, spinal fluid, lymph fluid, biopsy aspirates, ascites, fluidic extracts, solid or soft tissue, the external sections of the skin, respiratory, intestinal, and genitourinary tracts, tears, saliva, milk, tumors, organs, cell cultures and/or cell culture constituents. The sample may be obtained by any means, such as a syringe, adhesive strip, punch biopsy, microbiopsy, surgical removal, or the like.

In an embodiment the biological sample comprises a biopsy sample, tissue and peripheral blood. In an embodiment, the tissue sample may be a sample of skin cells obtained via a biopsy, a microbiopsy, or an adhesive strip that is configured to acquire the skin cells from the subject.

Biological samples from an armpit, the buttocks, perianal and gluteal regions, groin, and breast, including skin samples from these loci are contemplated. The sample may be from a lesional tissue (e.g., abscess, nodule or tunnel of a subject having or suspected to have HS) or may be from a healthy tissue or a tissue with a healthy phenotype.

### IL-17F As a Target in IL-17-Dependent Conditions

IL-17F is a unique target in hidradenitis suppurativa (HS) and psoriasis (PsO) and other inflammatory skin diseases. For example, IL-17F is specifically upregulated in skin and joint inflammation. In fact, IL-17F is more upregulated in HS than in PsO and is more abundant than IL-17A across HS lesions. IL-17F is also able to activate KCs independent of IL-17A. In view of this, inhibitors of IL-17F and/or inhibitors that block IL-17F at least as effectively as IL-17A, will be more effective for treatment of HS and PsO and other inflammatory skin diseases.

### IL-17 Inhibitors

IL-17 inhibitors as contemplated herein are not particularly limited and may comprise an antibody, antibody fragment, or a nanobody. The IL-17 inhibitors may be capable of modulating, e.g., blocking, inhibiting, reducing, antagonizing, neutralizing or otherwise interfering with IL-17, i.e., IL-17A and/or IL-17F mediated pro-inflammatory cytokine and/or chemokine production.

IL-17 inhibitors as contemplated herein may specifically bind to IL-17F, an IL-17F homodimer, IL-17A, an IL-17A homodimer, and/or a heterodimeric IL-17A/IL-17F complex.

As used herein, the term “antibody” may refer to immunoglobulin molecules and immunologically active portions of immunoglobulin (Ig) molecules, i.e., molecules that contain an antigen binding site that specifically binds (immunoreacts with) an antigen. For example, the antibody may be considered to react with one or more antigenic determinants of the desired antigen and not to react with other polypeptides or to bind at much lower affinity.

Antibodies include polyclonal, monoclonal, chimeric, dAb (domain antibody), single chain, Fab, Fab′, and F(ab′)2 fragments, and scFvs. Antibody fragments and antibodies which combine structures or features of one or more of the foregoing antibody types are also contemplated.

The basic antibody structural unit is known to the skilled person. Each antibody comprises a tetramer that is composed of two “light” chains (about 25 kDa each) and two “heavy” chains (about 50-70 kDa each). The amino-terminal portion of each chain includes a variable region of about 100 to 110 or more amino acids primarily responsible for antigen recognition. The carboxy-terminal portion of each chain defines a constant region. Human antibody molecules may relate to any of the classes IgG, IgM, IgA, IgE and IgD, which differ from one another by the nature of the heavy chain present in the molecule. In humans, the light chain may be a kappa chain or a lambda chain.

The term “monoclonal antibody” (mAb) as used herein, may refer to a single antibody species or molecule or to a population of antibody molecules that contain only one molecular species of antibody molecule, each consisting of a unique light chain gene product and a unique heavy chain gene product. The complementarity determining regions (CDRs) of the monoclonal antibody are identical in all the molecules of the population.

“Nanobody” or “VHH single domain antibody” as used herein are synonyms and may be used interchangeably. A VHH single domain antibody comprises an immunoglobulin single variable domain (“ISV”). An immunoglobulin single variable domain is an amino acid sequence that: (i) comprises an immunoglobulin fold or that, under suitable conditions (such as physiological conditions) is capable of forming an immunoglobulin fold (i.e. by folding), i.e. so as to form an immunoglobulin variable domain (such as, for example, a VH, VL or VHH domain); and that (ii) forms (or under such suitable conditions is capable of forming) an immunoglobulin variable domain that comprises a functional antigen binding activity (in the sense that it does not require an interaction with another immunoglobulin variable domain (such as a VH-VL interaction) to form a functional antigen binding site). Preferred examples of nanobodies or immunoglobulin single variable domains suitable for use in the invention include VHHs such as humanized VHH's or camelized VH's, such as camelized human VH, dAbs, and (single) domain antibodies. As contemplated herein, a nanobody may comprise or consist of a single ISV or VHH domain; as contemplated herein, a nanobody may also comprise or consistent of multiple ISV s or VHH domains and thereby a nanobody includes embodiments such as a VHH multiple domain antibody. A ntibodies and antibody fragments combined with ISVs, VHH structures, or nanobodies are also contemplated.

In preferred embodiments, the IL-17 inhibitor may selected from Bimekizumab, Secukinumab, Ixekizumab, Brodalumab, Netakimab, or Izokibep.

In a preferred embodiment, the IL-17 inhibitor may be an IL-17A- and/or IL-17F-inhibitor and may comprise an Fab, a VHH single domain antibody, or an scFV (single chain Fv fragment) having an ISV.

In an embodiment, the nanobody may have a mass of 35-45 kDa. The small size of nanobody inhibitors allows 10× higher tissue/serum ratio than convention mAb.

In an embodiment, the nanobody may be configured to specifically bind to IL-17A and IL-17F.

The nanobody may comprise a region which specifically binds IL-17F, a region which specifically binds human serum albumin, and a region which specifically binds IL-17A/F.

In embodiments, the nanobody may binds to IL17-F/F dimers at least as well as it binds IL-17A/A dimers.

In embodiments, the nanobody has an affinity for IL-17A/A, IL-17A/F, and/or IL-17F/F dimers of 0.037 nM or lower, preferably 0.004 nM or lower, as measured by surface plasmon resonance.

In embodiments, the nanobody is sonelokimab (SLK) ora derivative thereof. Sonelokimab is an IL-17A and IL-17F inhibiting nanobody with enhanced potential to penetrate and accumulate at sites of inflammation in deep and difficult to reach tissue structures such as dermal inflammatory and fibrotic HS lesions.

M odelling of sonelokimab PK and molecular PD from subcutaneous administartion to tissue penentration shows that unique advantages of the nanobody stem from its small size, enhanced absorption after sc. administration, and tissue penetration ability. It is expected that this will allow disease control at lower trough levels due to lower doses or longer injection intervals with improved benefit-risk ratio compared to mAb.

Modes of Administration

In the present disclosure, the term “administering” refers to providing a medicament to a subject. The method or route of administration is not particularly limited, and may be, for example, subcutaneous, intravenous, intraperitoneal, intramuscular, or transdermal. In a preferred embodiment, the administration is systemic (e.g., via injection). Subcutaneous injection is also contemplated. Subcutaneous injection may include injections by prefilled syringe, autoinjector or sc. injection of reconstituted lyophilized powder.

An “effective” or “therapeutically effective” amount of the medicament is typically administered. A n effective amount refers to an amount necessary (at dosages and for periods of time and for the means of administration) to achieve the desired therapeutic result. An effective amount of the medicament may vary according to factors such as the disease state, age, gender, and weight of the subject, and the ability of the medicament to elicit a desired response in the subject. An effective amount is also one in which any toxic or detrimental effects of the medicament are outweighed by the therapeutically beneficial effects.

Also disclosed herein is a method of treating a subject having or at risk of having hidradenitis suppurativa, which comprises administering an effective amount of an agent configured to inhibit IL-17F present in the dermis of an inflammatory skin lesion of the subject, wherein the effective amount is the same dosage required to effectively treat psoriasis with the same agent.

In an embodiment, such a method may comprise administering the effective amount to a subject having an assayed lesional IL-17F/non-lesional IL-17F protein ratio of 2 or higher, preferably 10 or higher, more preferably 2-50, and most preferably 10-30.

In an embodiment, such a method may comprise administering the effective amount to a subject having an assayed lesional IL-17F/non-lesional IL-17 mRNA ratio of 5 or higher, preferably 10 or higher, more preferably 5-500, and most preferably 10-300.

In an embodiment, the agent configured to inhibit IL-17F may comprise an antibody, an antibody fragment, or a nanobody. In an embodiment, the agent may comprise a nanobody and the nanobody may have a mass of 35-45 kDa.

### Kits

Kits for diagnosing IL-17-dependent conditions, identifying supper-responders, and/or simply measuring the biomarkers are contemplated.

For example, contemplated herein is a kit for identifying a subject at risk or in need of treatment for an IL-17-dependent condition, which kit comprises a reagent or reagents for measuring the level or expression of one or more of: IL6, PLA2G2A, IL19, PI3, CST7, IL17A, IL17F, GH1, MZB1, IL1B, IFNG, TNC, CXCL9, SLAMF7, VEGFA, 1L17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, CXCL13, PCDH1, BOC, MEPE, ADAM23, THOP1, IL1RL2, RCOR1, and EDAR.

In an embodiment, the kit may further comprise packaged components for obtaining a biological sample from a subject, preferably for obtaining a skin sample, a blood sample, or other body fluid sample. In certain embodiments, such a kit may comprise an adhesive strip for obtaining a skin sample or packaged components for obtaining a punch biopsy, microbiopsy, or surgical specimen.

A kit for treating an IL-17-dependent dermatological condition is also contemplated herein, wherein the kit may comprise a pharmaceutical composition comprising an inhibitor of IL-17A and/or IL-17F, and a reagent or reagents for measuring the level or expression of one or more of: IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, CXCL13, PCDH1, BOC, MEPE, ADAM23, THOP1, IL1RL2, RCOR1, and/or EDAR, in a sample taken from a subject.

In the present disclosure, a “kit” may refer to those materials required to carry out an evaluation, including collecting and safely disposing of a sample. For example, a kit may include an adhesive strip used to obtain a skin sample. A kit may alternatively include materials for collecting bodily fluids such as blood and, if needed, separating blood into component parts such as plasma and red blood cells. Such materials could include, for example, a syringe or sponge. The kit may also include materials such as reagents (buffers, blocking agents, etc.), assay cartridges, and the like. A kit may also include written instructions for sample collection and testing.

Alternatively, a kit may include those materials required for administering a medicament. Such a kit may includethe medicament in the form of, for example, pills, tablets, cremes, medicated foods or drink, and injectable liquid medicine. The kit may also include administration devices such as syringes or pens pre-filled with the medicament. The kit may also include materials for safely disposing of the administration devices, and written instructions for use.

Also disclosed herein is a kit for identifying a subject at risk or in need of treatment with an IL-17F inhibitor, which kitcomprisesan adhesive strip configured to obtain a testskin sample; a reagent for measuring the level or expression of IL17F, and optionally a reagent for measuring the level or expression of IL17A.

Also disclosed herein is a kit for treating an IL-17-dependent dermatological condition, wherein the kit comprises a pharmaceutical composition, a reagent for measuring the level or expression of IL17F in a sample taken from a subject, and optionally a reagent for measuring the level or expression of IL17A in a sample taken from a subject, wherein the pharmaceutical composition comprises an inhibitor of IL-17F.

In an embodiment, contemplated herein is such a kit, wherein the sample taken from the subject to measure the level of IL-17F is a sample of skin cells and the kit further comprises an adhesive strip configured to acquire the sample of skin cells from the subject.

In an embodiment, contemplated herein is such a kit wherein the IL-17-dependent dermatological condition is hidradenitis suppurativa.

Also disclosed herein is a kit for identifying a subject at risk of having HS or in need of treatment with an IL-17A and/or IL-17F inhibitor, which kit comprises: an adhesive strip configured to obtain a test skin sample; and at least one reagent for measuring the level or expression of at least one biomarker in a sample taken from the subject, wherein the biomarker comprises one or more of CCL20, CXCL1, CXCL8, CCL2, CCL3, CCL4, CCL5, CCL7, PDFGAA, or CSF3.

Also disclosed herein is a kit for treating an IL-17-dependent dermatological condition, wherein the kit comprises a pharmaceutical composition and reagents for measuring the level or expression of at least one biomarker in a sample taken from a subject, wherein the pharmaceutical composition comprises an inhibitor of IL-17A and/or IL17F, and the biomarker comprises one or more of CCL20, CXCL1, CXCL8, CCL2, CCL3, CCL4, CCL5, CCL7, PDFGAA, orCSF3.

In an embodiment, a kit as above is contemplated, wherein the sample taken from the subject is a sample of skin cells and the kit may further comprise an adhesive strip configured to acquire the sample of skin cells from the subject.

In an embodiment, a kit as above is contemplated, wherein the IL-17-dependent dermatological condition is hidradenitis suppurativa.

### EXAMPLES

### Example 1

### Inflammatory Activity Based on IL-17A and IL-17F Expression Observed to Occur Maximally Around Dermal Tunnels in HS.

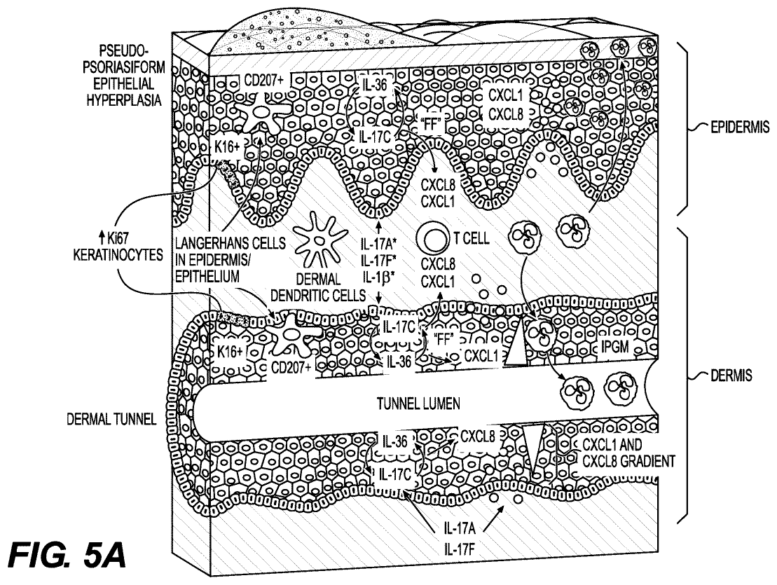

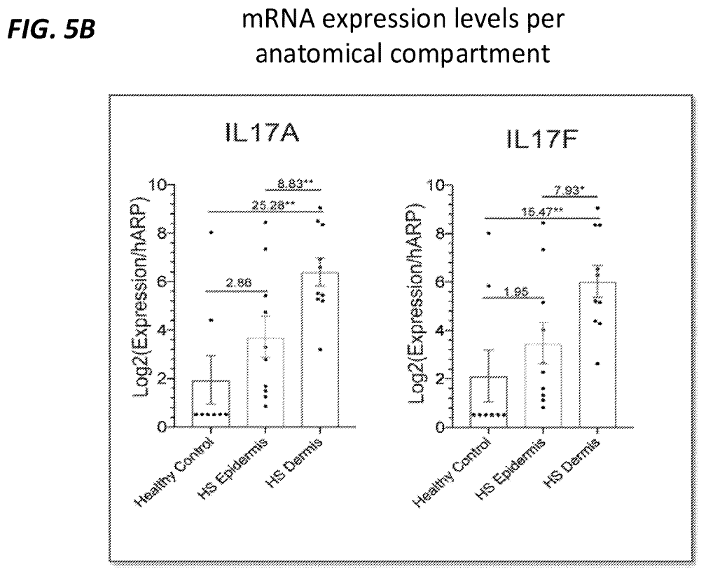

Quantitative RT-PCR was performed on HS samples from the dermis and epidermis as well as on healthy controls. Higher levels of IL-17A and IL-17F mRNA were observed in the dermal compared to the epidermal compartment in HS lesions. The results indicate that maximum inflammatory activity occurs around dermal tunnels. See FIG. 5B.

### Example 2

### Identification of IL-17i-Relevant HS Biomarkers and the Characterization of Specific Targeted Molecule Properties.

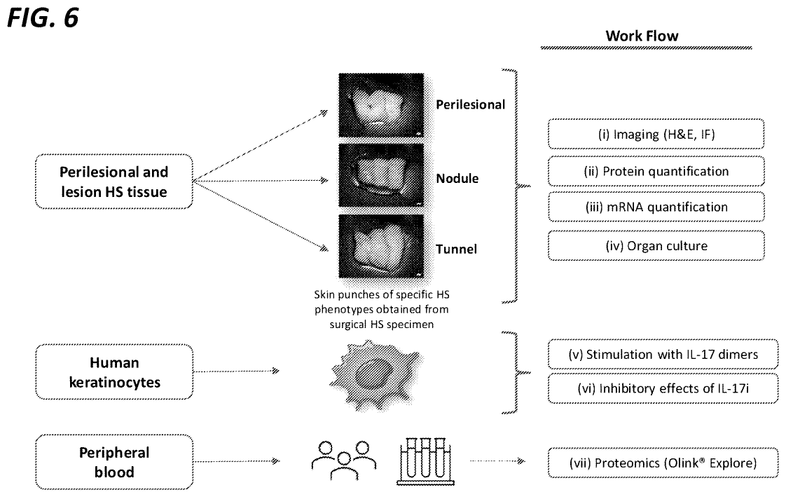

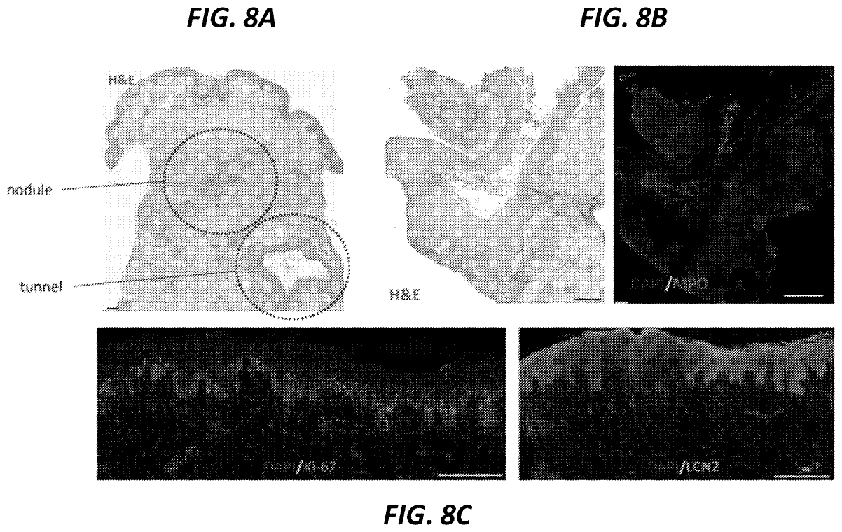

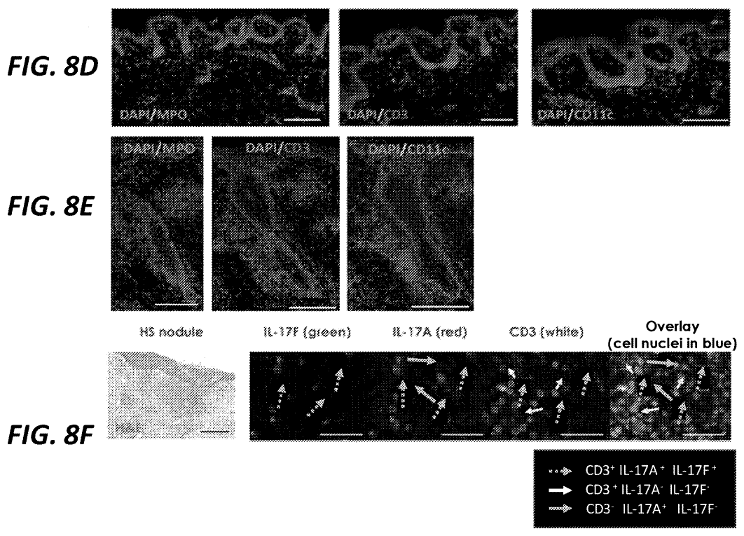

Punch biopsies of specific HS phenotypes (perilesional tissue, nodule-containing tissue, tunnel-containing tissue) were obtained from larger surgical specimen of patients with a diagnosis of HS (disease severity Hurley II-III) undergoing surgery for HS. Biopsies were first subjected to imaging [H & E staining e.g., to identify nodules and tunnels, multi-channel immunofluorescence (IF) to characterize keratinocyte activation and the immune cell infiltrate (FIG. 6—Work Flow at (i)); see also FIGS. 8A-8F). Biopsies were then subjected to protein lysate and mRNA extraction which were further analyzed by multi-cytokine array or ELISA and bulk RNAseq and quantitative RT-PC R analyses (FIG. 6—Work Flow at (ii) and (iii); see also Table 1).

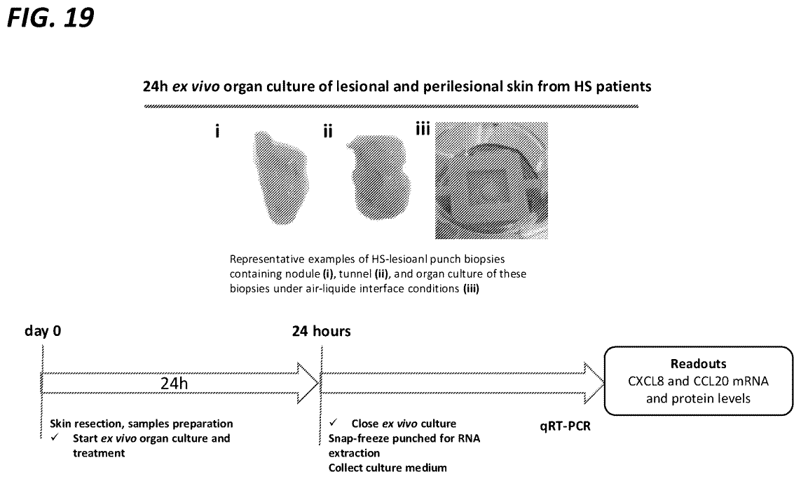

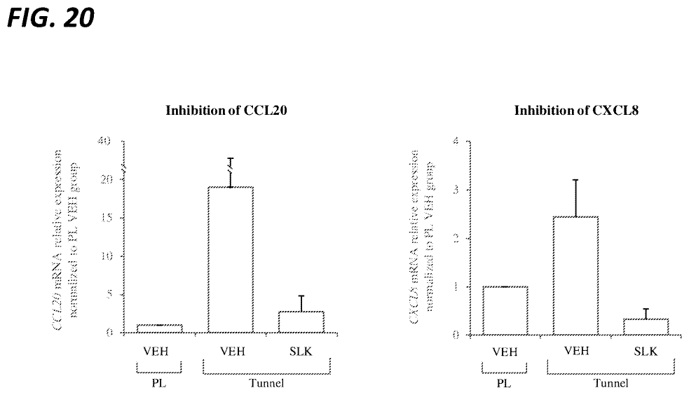

Selected perilesional and tunnel-containing biopsies were used for air-liquid interface organ culture to test the penetration and specific anti-inflammatory effects of IL-17-inhibiting (IL-17i) therapeutic molecules (FIG. 6—Work Flow at (iv); see also FIG. 19 and FIG. 20).

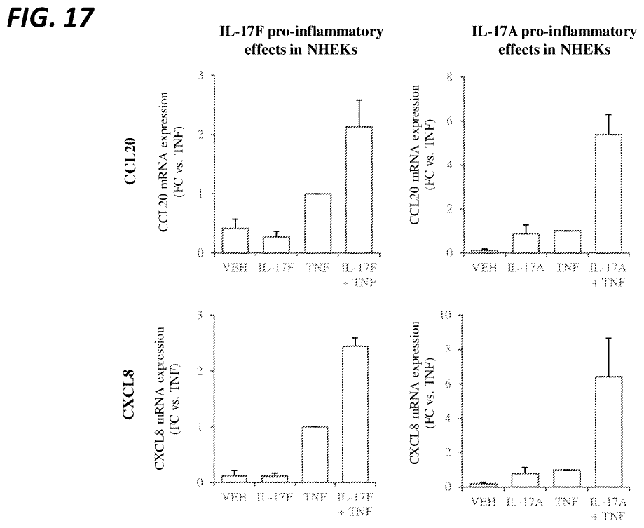

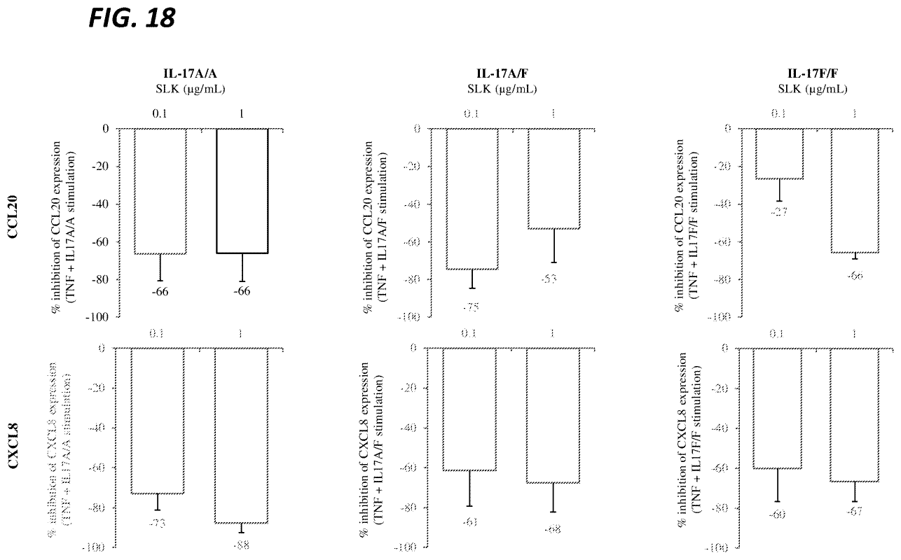

KCs were then exposed to different IL-17 dimers to test the specific pro-inflammatory effects of IL-17A and IL-17F (FIG. 6—Work Flow at (v)). Different IL-17i therapeutic molecules were added to evaluate the specific inhibitory potential across different concentrations of these molecules (FIG. 6—Work Flow at (vi); see also FIGS. 17 and 18).

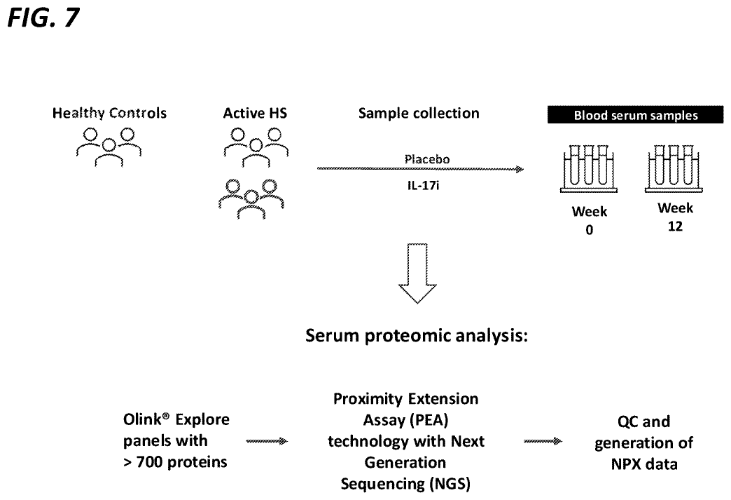

Finally, peripheral blood from patients with HS was collected before and after treatment with an IL-17 inhibitor (IL17i), alongside collection of peripheral blood from healthy controls. Proteomics (Olink®) was used to further identify biomarkers for HS (FIG. 6—Work Flow at (vii); see also FIGS. 7, 21, 22; and Table 1). In particular, a total of 502 serum samples from 233 subjects with HS were analysed using the Olink® Explore platform, i.e., the Olink® Explore 384 Cardiometabolic and Olink® Explore 384 Inflammation panels, with together comprise>700 proteins linked to inflammation and/or metabolic disease. TheOlink® Explore platform uses Proximity Extension Assay (PEA) technology coupled to a readout methodology based on Next Generation Sequencing (NGS). The PEA technology is a dual-recognition immunoassay in which matched antibody pairs carrying unique DNA-tags (PEA probes) bind to a protein target. Upon target binding, the attached oligonucleotides are brought into proximity and hybridize. The newly generated oligonucleotide sequence is then amplified by PCR and measured by NGS. Quality control (QC) and data normalization was performed using internal and external controls.

Notably, the expression of the following biomarkers was observed to be elevated in HS: IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13; further, expression of the following biomarkers was observed to be reduced in HS: PCDH1, BOC, MEPE, ADAM23, THOP1, IL1RL2, RCOR1, and EDAR. The identified biomarkers and relevant information relating to each is summarized in Table 1.

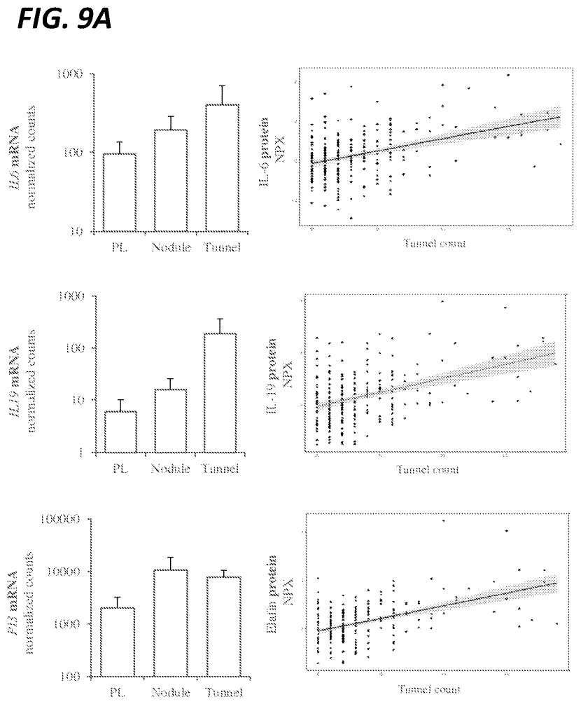

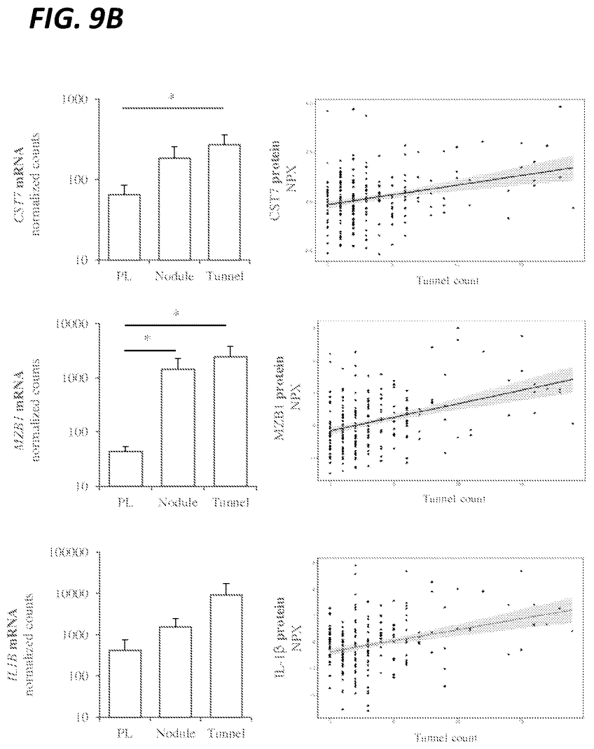

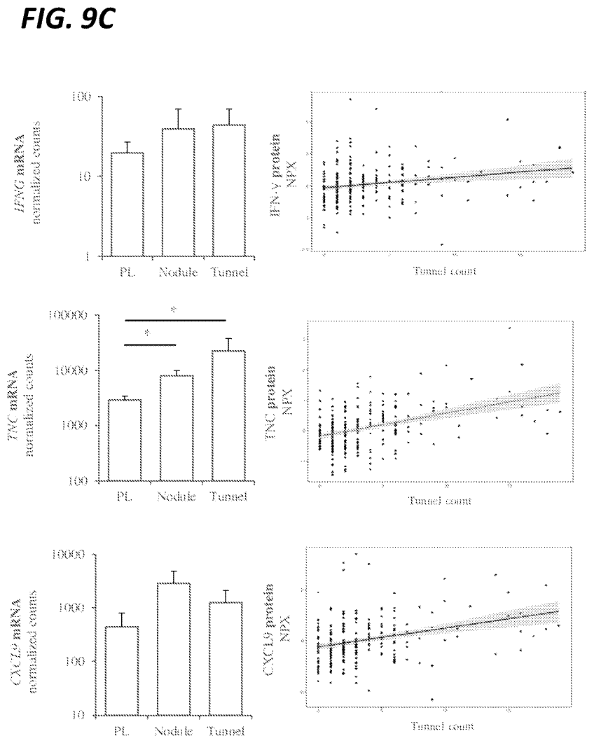

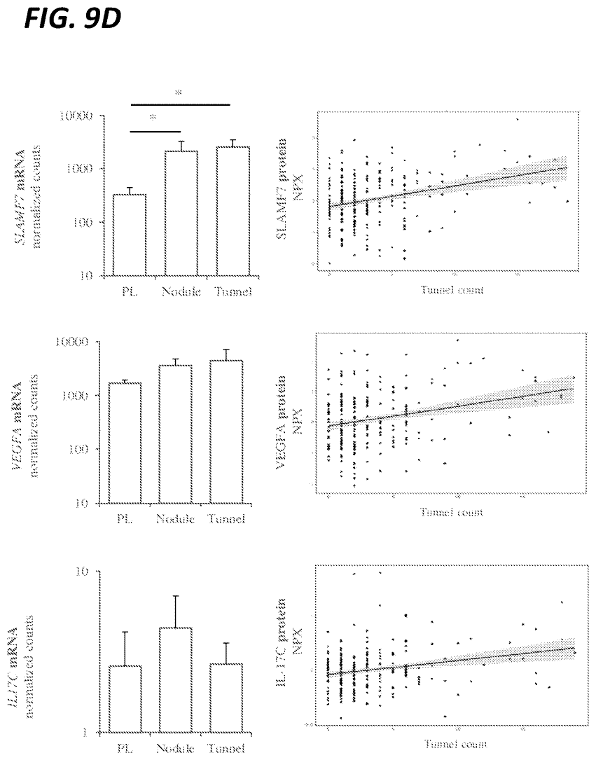

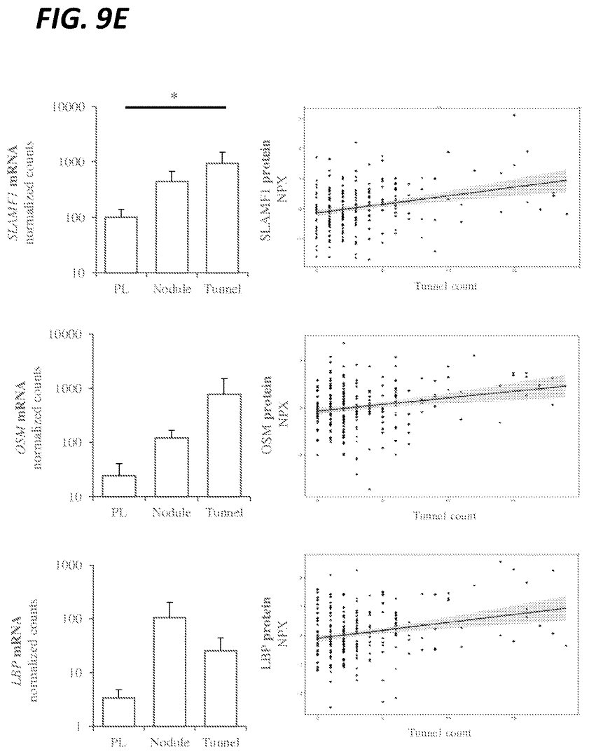

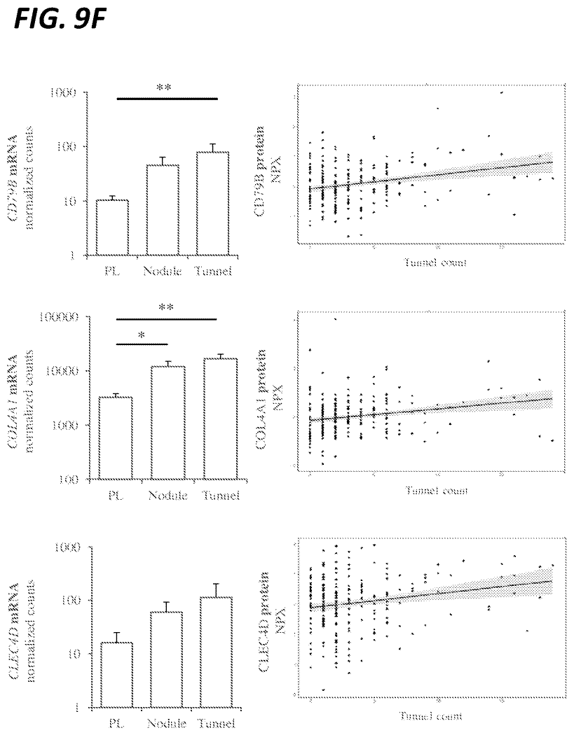

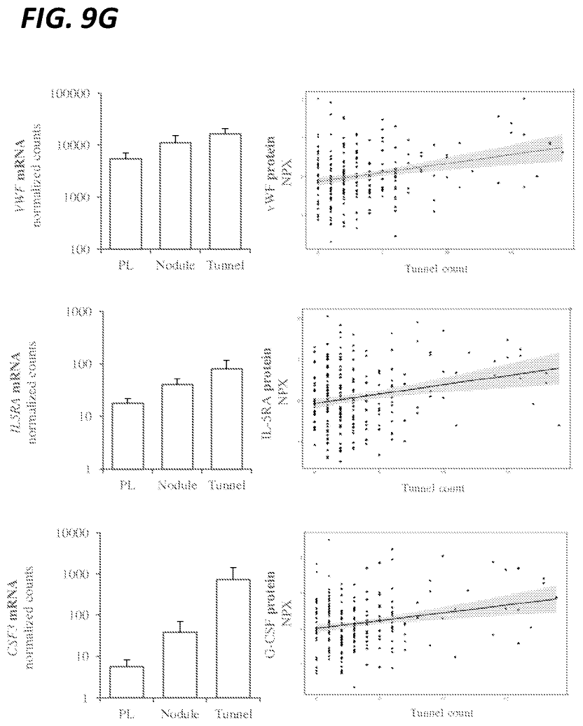

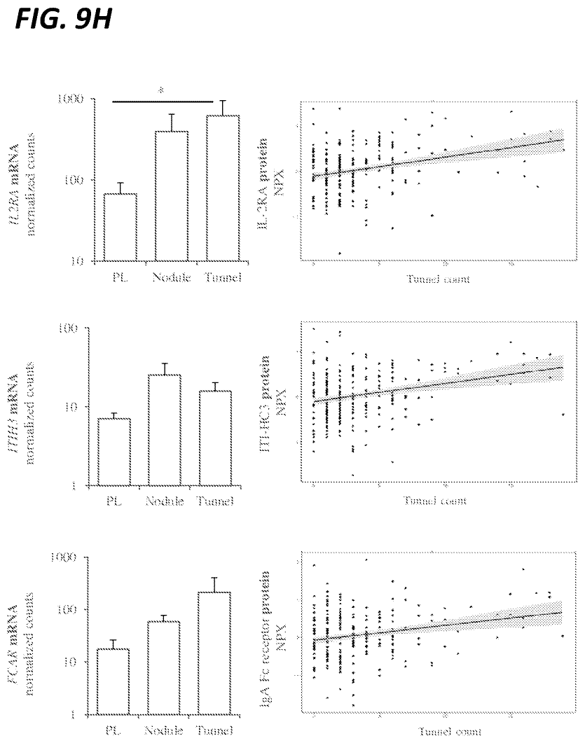

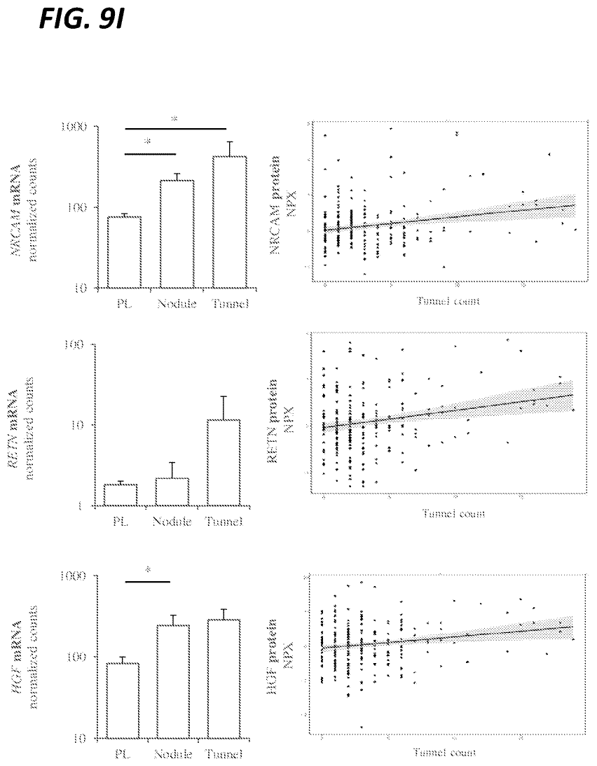

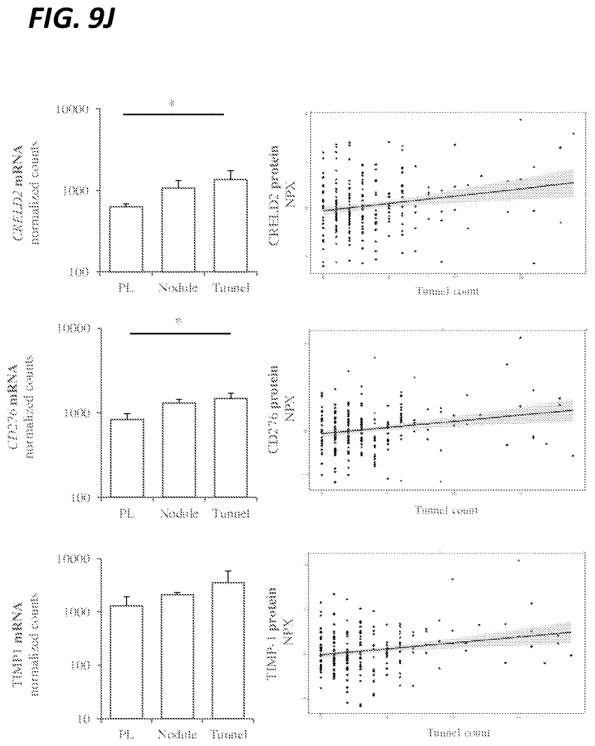

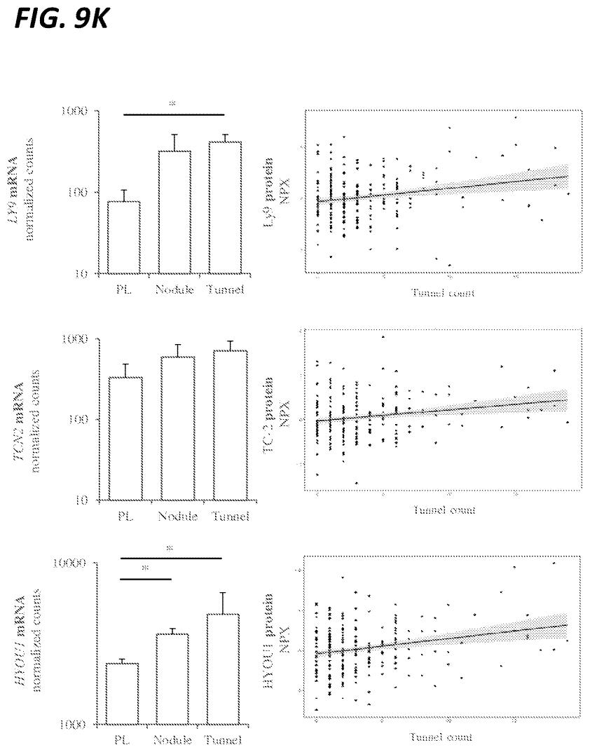

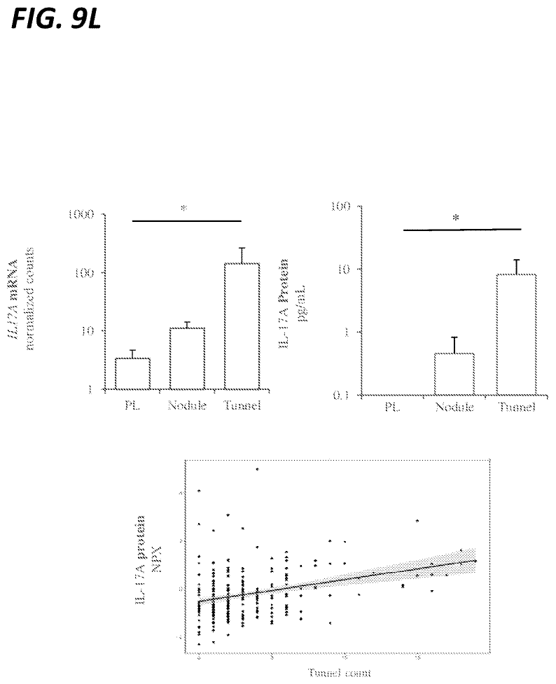

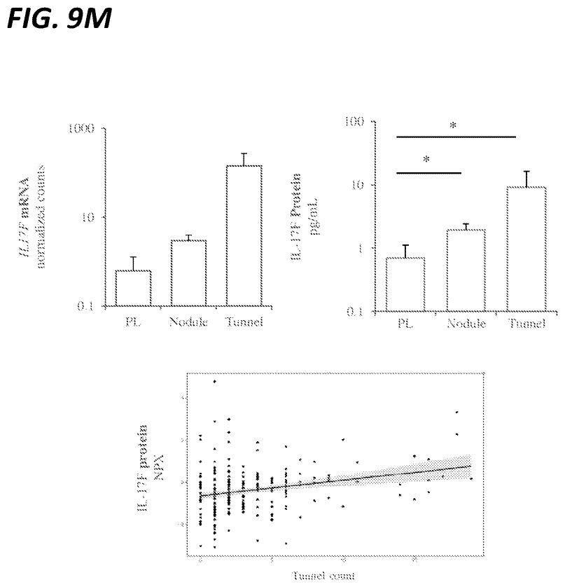

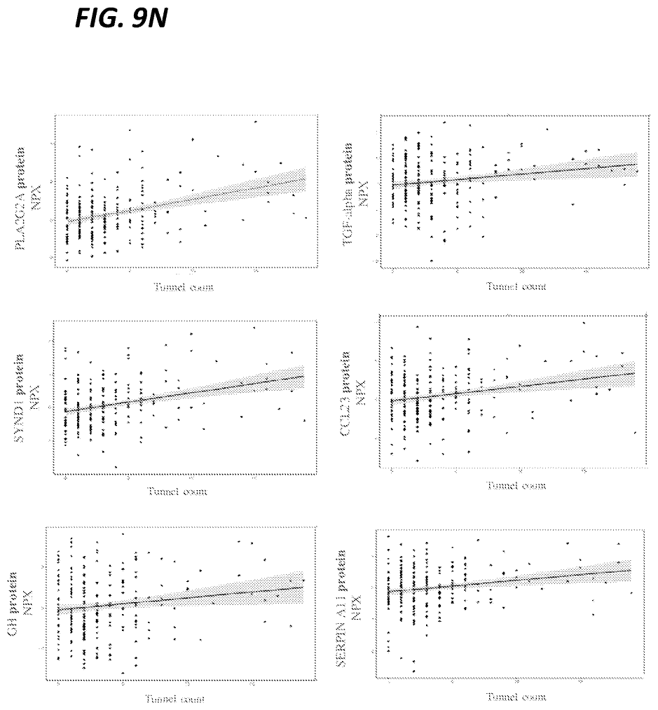

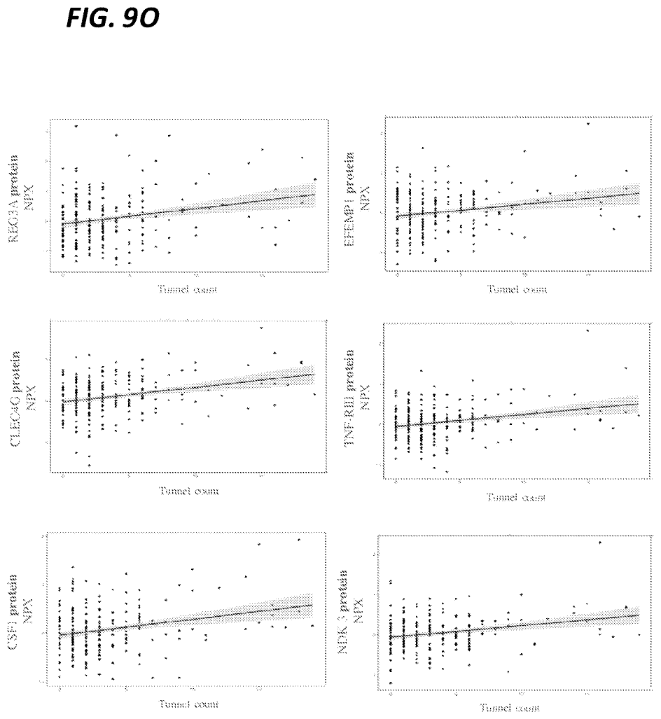

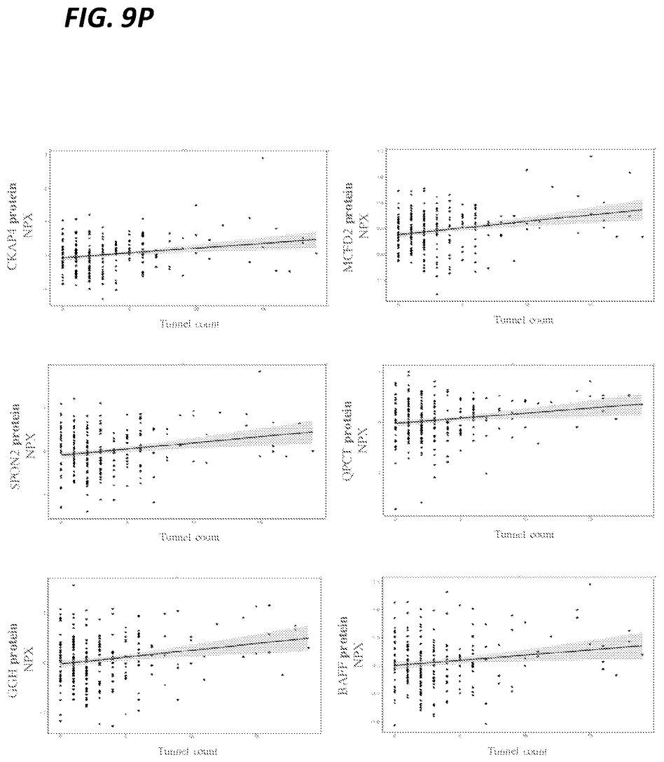

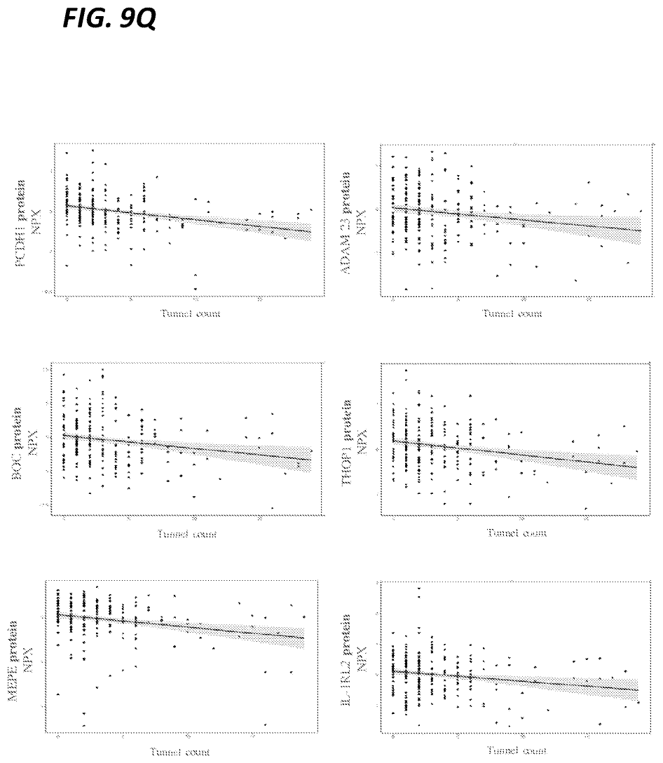

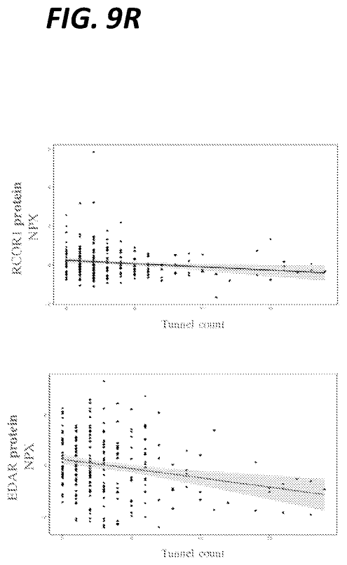

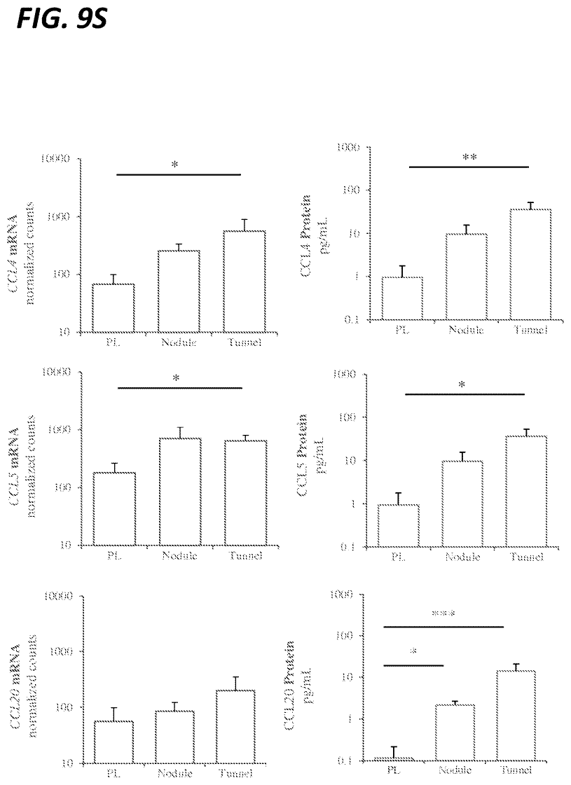

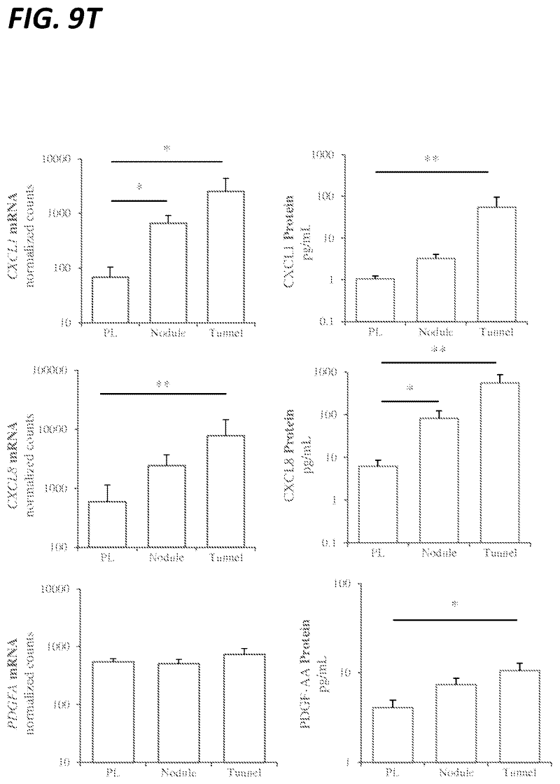

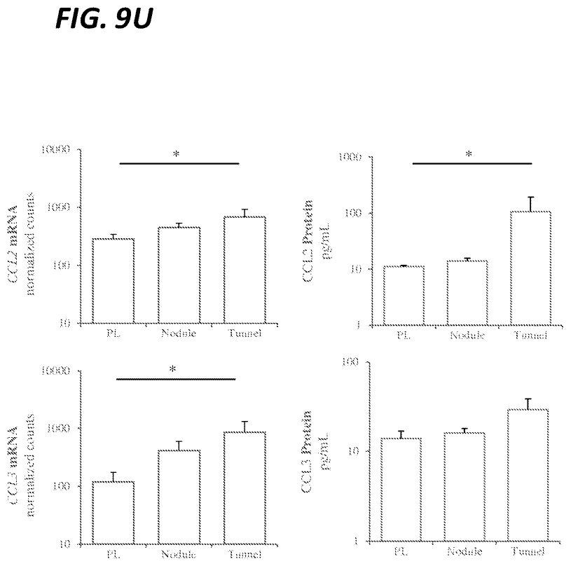

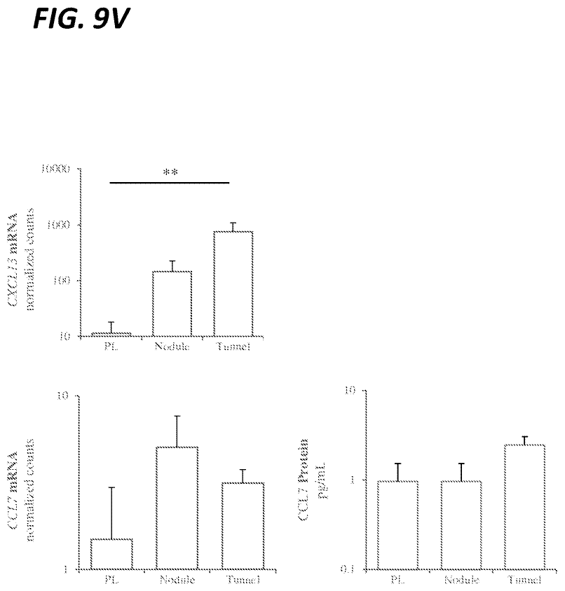

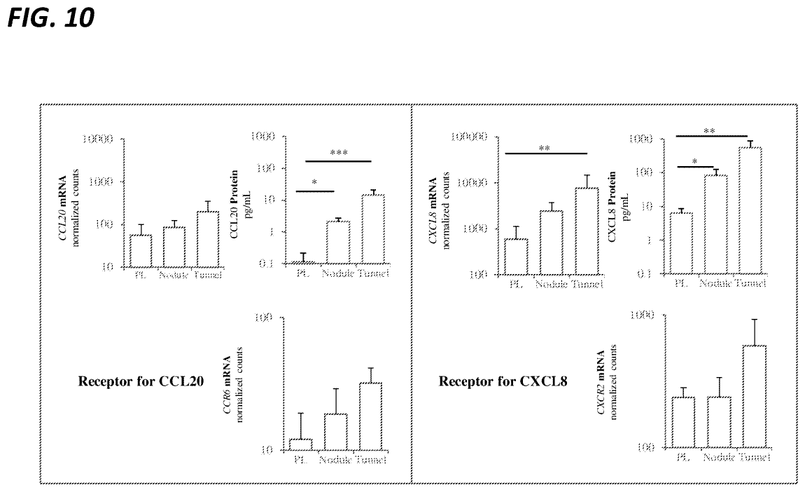

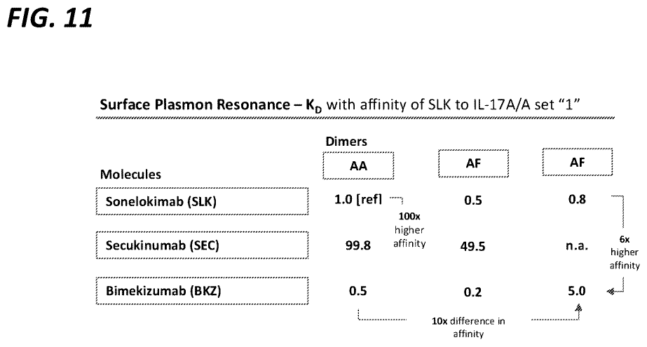

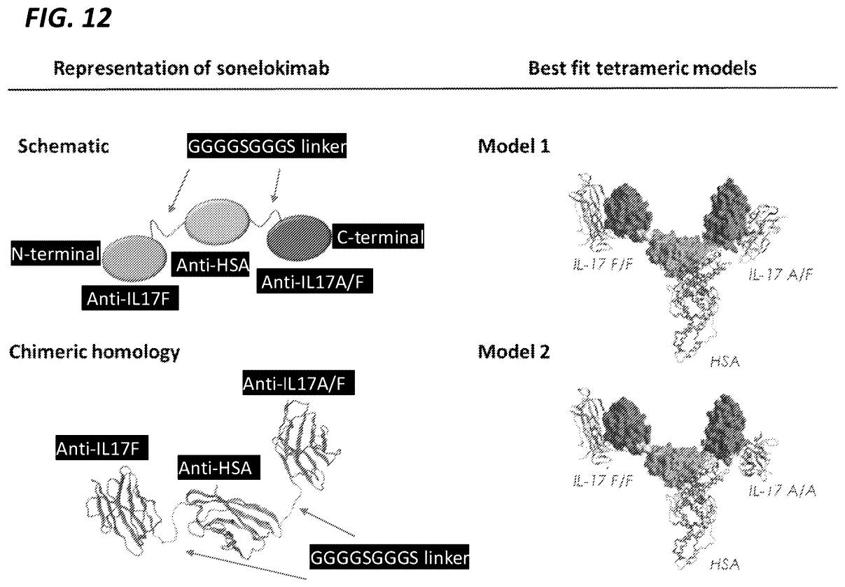

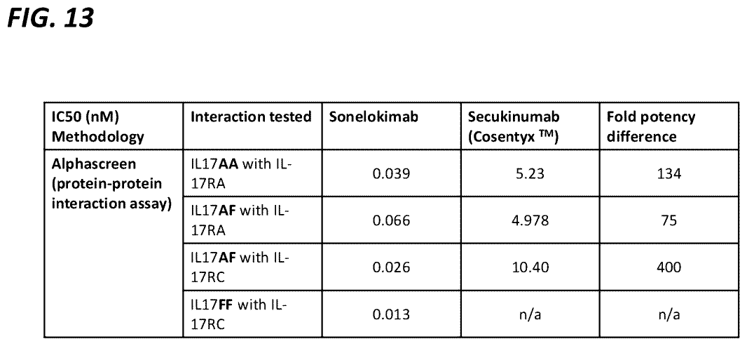

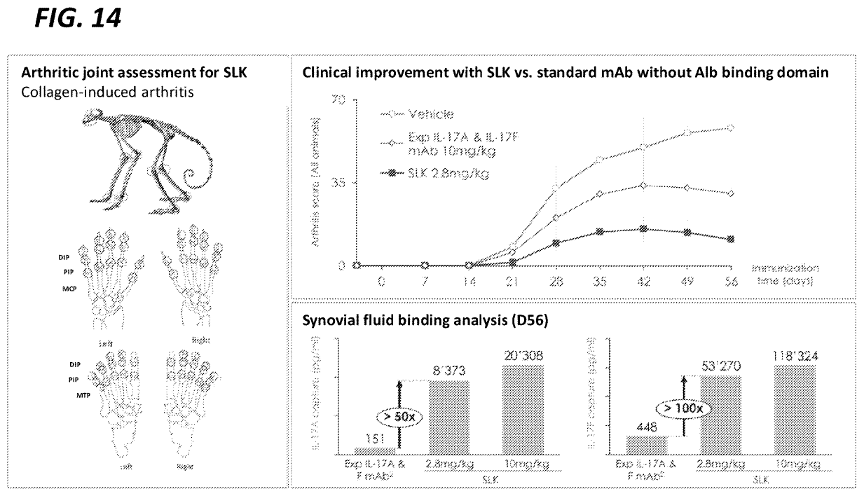

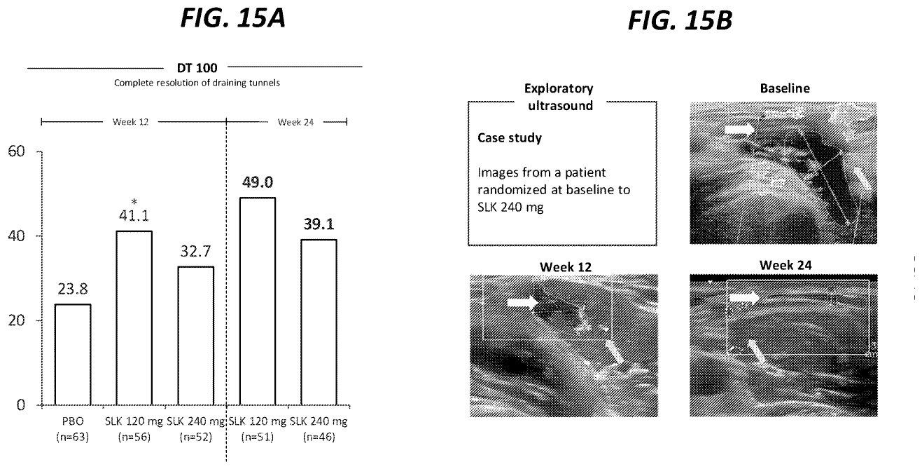

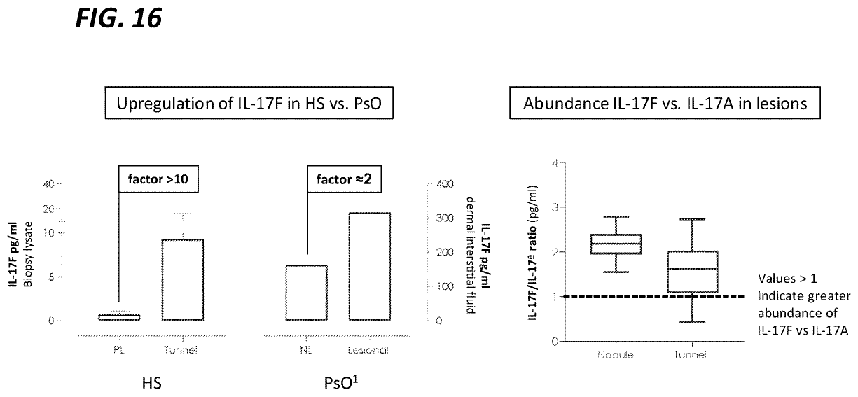

TABLE 1
HS Biomarkers

| | | UniProt code/ | | | | Inventive Aspects and Features | | | |
|---|---|---|---|---|---|---|---|---|---|
| | | Exemplary Amino | | | Known Functions | Compartment with | Example methods of | | |
| Biomarker | Protein symbol | Acid Sequence | Gene symbol | Alias symbols | Include | aberrant expression | sample collection | Data/Technical Effect | |
| Interleukin-6 (IL6) | IL-6 | P05231/ | IL6 | BSF2 | B-cell activation; T-cell/ | Peripheral | Blood test | Protein | FIG. 9A |
| | | SEQ ID | | HGF | Th17 activation; | blood | | (blood proteomics): | |
| | | NO: 1 | | HSF | Angiogenesis | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.13; | |
| | | | | | | | | Adjusted P: | |
| | | | | | | | | 6.92E−07) | |
| Phospholipase A2, | PLA2G2A | P14555/ | PLA2G2A | PLA2B | Epithelial regeneration; | Peripheral | Blood test | Protein | FIG. 9N |
| membrane | | SEQ ID | | PLA2L | Antimicrobial defense | blood | | (blood proteomics): | |
| associated (PLA2G2A) | | NO: 2 | | RASF-A | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.12; | |
| | | | | | | | | Adjusted P: | |
| | | | | | | | | 8.30E−06) | |
| Interleukin-19 (IL19) | IL-19 | Q9UHD0/ | IL19 | IL-19 | Angiogenesis; | Peripheral | Blood test | Protein | FIG. 9A |
| | | SEQ ID | | MDA1 | Neutrophil activation; | blood | | (blood proteomics): | |
| | | NO: 3 | | ZMDA1 | Epithelial regeneration; | | | Elevated vs. | |
| | | | | IL-10C | Angiogenesis | | | healthy | |
| | | | | NG.1 | | | | controls | |
| | | | | | | | | (FDR < 0.05); | |
| | | | | | | | | Correlation | |
| | | | | | | | | with dT count | |
| | | | | | | | | (Slope: 0.11; | |
| | | | | | | | | Adjusted P: | |
| | | | | | | | | 5.49E−07) | |
| Elafin (PI3) | Elafin | P19957/ | PI3 | ESI | Neutrophil | Peripheral | Blood test | Protein | FIG. 9A |
| | | SEQ ID | | SKALP | activation/degradation; | blood | | (blood proteomics): | |
| | | NO: 4 | | ELAFIN | Antimicrobial activity | | | Elevated vs. | |
| | | | | WAP3 | | | | healthy | |
| | | | | WFDC14 | | | | controls | |
| | | | | cementoin | | | | (FDR < 0.05); | |
| | | | | | | | | Correlation | |
| | | | | | | | | with dT count | |
| | | | | | | | | (Slope: 0.10; | |
| | | | | | | | | Adjusted P: | |
| | | | | | | | | 6.28E−09) | |
| Cystatin-F (CST7) | CST7 | 076096/ | CST7 | Leukocystatin | NK cell activation; | Lesional skin | Blood test, | Protein | FIG. 9B |
| | | SEQ ID | | Cystatin-7 | Granulocyte regulation | sample and | tape strip, punch | (blood proteomics): | |
| | | NO: 5 | | CMAP | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.10; | |
| | | | | | | | | Adjusted P: | |
| | | | | | | | | 9.91E−04) | |
| | | | | | | | | mRNA (RNA-seq): | |
| | | | | | | | | lesional vs | |
| | | | | | | | | perilesional | |
| | | | | | | | | (P < 0.05) | |
| Interleukin-17A | IL-17A | Q16552/ | IL17A | IL-17 | T-cell/Th17 activation; | Lesional skin | Blood test, | Protein | FIG. 9L |
| (IL17A) | | SEQ ID | | | Neutrophil activation | sample and | tape strip, punch | (blood proteomics): | |
| | | NO: 6 | | | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.09; | |
| | | | | | | | | Adjusted P: | |
| | | | | | | | | 3.91E−04) | |
| | | | | | | | | mRNA (RNA-seq): | |
| | | | | | | | | lesional vs | |
| | | | | | | | | perilesional | |
| | | | | | | | | (P < 0.05) | |
| | | | | | | | | Protein: | |
| | | | | | | | | lesional vs | |
| | | | | | | | | perilesional | |
| | | | | | | | | (P < 0.05) | |
| Interleukin-17F | IL-17F | Q96PD4/ | IL17F | ML-1 | T-cell/Th17 activation; | Lesional skin | Blood test, | Protein | FIG. 9M |
| (IL17F) | | SEQ ID | | ML1 | Neutrophil activation | sample and | tape strip, punch | (blood proteomics): | |
| | | NO: 7 | | | | peripheral | biopsies, microbiopsy | Correlation | |
| | | | | | | blood | and any type of | with dT count | |
| | | | | | | | surgical specimen | (Slope: 0.07; | |
| | | | | | | | | Adjusted P: | |
| | | | | | | | | 3.35E−03) | |
| | | | | | | | | Protein: | |
| | | | | | | | | lesional vs | |
| | | | | | | | | perilesional | |
| | | | | | | | | (P < 0.05) | |
| Somatotropin (GH1) | GH | P01241/ | GH1 | GH-N | Hormone response | Peripheral | Blood test | Protein | FIG. 9N |
| | | SEQ ID | | GHN | | blood | | (blood proteomics); | |
| | | NO: 8 | | GH | | | | Elevated vs. | |
| | | | | hGH-N | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.09; | |
| | | | | | | | | Adjusted P: | |
| | | | | | | | | 3.30E−02) | |
| Marginal zone B- and | MZB1 | Q8WU39/ | MZB1 | PACAP | B-cell activation | Lesional skin | Blood test, | Protein | FIG. 9B |
| B1-cell-specific | | SEQ ID | | MGC29506 | | sample and | tape strip, punch | (blood proteomics): | |
| protein (MZB1) | | NO: 9 | | HSPC190 | | peripheral | biopsies, microbiopsy | Elevated vs. | |
| | | | | pERp1 | | blood | and any type of | healthy | |
| | | | | MEDA-7 | | | surgical specimen | controls | |
| | | | | | | | | (FDR < 0.05); | |
| | | | | | | | | Correlation | |
| | | | | | | | | with dT count | |
| | | | | | | | | (Slope: 0.08; | |
| | | | | | | | | Adjusted P: | |
| | | | | | | | | 2.14E−06) | |
| | | | | | | | | mRNA (RNA-seq): | |
| | | | | | | | | lesional vs | |
| | | | | | | | | perilesional | |
| | | | | | | | | (P < 0.05) | |
| Interleukin-1 beta | IL-1β | P01584/ | IL1B | IL1F2 | T-cell/Th17 activation; | Peripheral | Blood test | Protein | FIG. 9B |
| (IL1B) | | SEQ ID | | IL-1B | B-cell activation; | blood | | (blood proteomics): | |
| | | NO: 10 | | IL1-BETA | Epithelial regeneration | | | Correlation | |
| | | | | | | | | with dT count | |
| | | | | | | | | (Slope: 0.08; | |
| | | | | | | | | Adjusted P: | |
| | | | | | | | | 1.82E−04) | |
| IFN-γ (IFNG) | IFN-γ | P01579/ | IFNG | — | Innate immunity; B-cell | Peripheral | Blood test | Protein | FIG. 9C |
| | | SEQ ID | | | activation; T-cell | blood | | (blood proteomics): | |
| | | NO: 11 | | | activation Immune cell | | | Elevated vs. | |
| | | | | | extravasation; NK cell, | | | healthy | |
| | | | | | CD8+ activation | | | controls | |
| | | | | | | | | (FDR < 0.05); | |
| | | | | | | | | Correlation | |
| | | | | | | | | with dT count | |
| | | | | | | | | (Slope: 0.08 | |
| | | | | | | | | Adjusted P: | |
| | | | | | | | | 2.03E−02) | |
| Tenascin C (TNC) | TNC | P24821/ | TNC | TN | Neurogenesis | Lesional skin | Blood test, | Protein | FIG. 9C |
| | | SEQ ID | | MGC167029 | | sample and | tape strip, punch | (blood proteomics): | |
| | | NO: 12 | | | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.07; | |
| | | | | | | | | Adjusted P: | |
| | | | | | | | | 5.49E−07) | |
| | | | | | | | | mRNA (RNA-seq): | |
| | | | | | | | | lesional vs | |
| | | | | | | | | perilesional | |
| | | | | | | | | (P < 0.05) | |
| C-X-C motif | CXCL9 | Q07325/ | CXCL9 | SCYB9 | Immune cell | Peripheral | Blood test | Protein | FIG. 9C |
| chemokine ligand 9 | | SEQ ID | | Humig | extravasation; T-cell | blood | | (blood proteomics): | |
| (CXCL9) | | NO: 13 | | crg-10 | activation | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.07; | |
| | | | | | | | | Adjusted P: | |
| | | | | | | | | 1.04E−03) | |
| SLAM family | SLAMF7 | Q9NQ25/ | SLAMF7 | CRACC | NK cell activation; T- | Lesional skin | Blood test, | Protein | FIG. 9D |
| member 7 (SLAMF7) | | SEQ ID | | 19A | cell activation | sample and | tape strip, punch | (blood proteomics): | |
| | | NO: 14 | | CS1 | | peripheral | biopsies, microbiopsy | Elevated vs. | |
| | | | | CD319 | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.07; | |
| | | | | | | | | Adjusted P: | |
| | | | | | | | | 1.78E−03) | |
| | | | | | | | | mRNA (RNA-seq): | |
| | | | | | | | | lesional vs | |
| | | | | | | | | perilesional | |
| | | | | | | | | (P < 0.05) | |
| Vascular endothelial | VEGFA | P15692/ | VEGFA | VEGF | Angiogenesis; | Peripheral | Blood test | Protein | FIG. 9D |
| growth factor A, long | | SEQ ID | | | Endothelial cell growth | blood | | (blood proteomics): | |
| form (VEGFA) | | NO: 15 | | | regulation | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.06; | |
| | | | | | | | | Adjusted P: | |
| | | | | | | | | 2.91E−03) | |
| Interleukin-17C | IL-17C | Q9POM4/ | IL17C | IL-17C | Epithelial immune | Peripheral | Blood test | Protein | FIG. 9D |
| (IL17C) | | SEQ ID | | CX2 | regulation | blood | | (blood proteomics): | |
| | | NO: 16 | | IL-21 | | | | Correlation | |
| | | | | MGC126884 | | | | with dT count | |
| | | | | MGC138401 | | | | (Slope: 0.06; | |
| | | | | | | | | Adjusted P: | |
| | | | | | | | | 1.36E−02) | |
| Signaling | SLAMF1 | Q13291/ | SLAMF1 | CD150 | T-cell activation | Lesional skin | Blood test, | Protein | FIG. 9E |
| lymphocytic | | SEQ ID | | | | sample and | tape strip, punch | (blood proteomics): | |
| activation molecule | | NO: 17 | | | | peripheral | biopsies, microbiopsy | Elevated vs. | |
| (SLAMF1) | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.06; | |
| | | | | | | | | Adjusted P: | |
| | | | | | | | | 8.27E−03) | |
| | | | | | | | | mRNA (RNA-seq): | |
| | | | | | | | | lesional vs | |
| | | | | | | | | perilesional | |
| | | | | | | | | (P < 0.05) | |
| Syndecan 1 (SDC1) | SYND1 | P18827/ | SDC1 | CD138 | Angiogenesis; Epithelial | Peripheral | Blood test | Protein | FIG. 9N |
| | | SEQ ID | | syndecan | regeneration | blood | | (blood proteomics): | |
| | | NO: 18 | | SYND1 | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.06; | |
| | | | | | | | | Adjusted P: | |
| | | | | | | | | 1.69E−03) | |
| Oncostatin-M (OSM) | OSM | P13725/ | OSM | MGC20461 | Epithelial immune | Peripheral | Blood test | Protein | FIG. 9E |
| | | SEQ ID | | | regulation; Epithelial | blood | | (blood proteomics): | |
| | | NO: 19 | | | regeneration | | | Correlation | |
| | | | | | | | | with dT count | |
| | | | | | | | | (Slope: 0.06; | |
| | | | | | | | | Adjusted P: | |
| | | | | | | | | 7.07E−03) | |
| Lipopolysaccharide- | LBP | P18428/ | LBP | BPIFD2 | Innate immunity | Peripheral | Blood test | Protein | FIG. 9E |
| binding protein (LBP) | | SEQ ID | | | | blood | | (blood proteomics): | |
| | | NO: 20 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.05; | |
| | | | | | | | | Adjusted P: | |
| | | | | | | | | 1.39E−02) | |
| Regenerating islet- | REG3A | Q06141/ | REG3A | HIP | Keratinocyte | Peripheral | Blood test | Protein | FIG. 9O |
| derived protein 3- | | SEQ ID | | REG-III | proliferation regulation | blood | | (blood proteomics): | |
| alpha (REG3A) | | NO: 21 | | REG3 | | | | Correlation | |
| | | | | PBCGF | | | | with dT count | |
| | | | | PAP1 | | | | (Slope: 0.05; | |
| | | | | | | | | Adjusted P: | |
| | | | | | | | | 3.41E−02) | |
| B-cell antigen | CD79B | P40259/ | CD79B | B29 | B-cell activation | Lesional skin | Blood test, | Protein | FIG. 9F |
| receptor complex- | | SEQ ID | | Ig-beta | | sample and | tape strip, punch | (blood proteomics): | |
| associated protein | | NO: 22 | | Igbeta | | peripheral | biopsies, microbiopsy | Elevated vs. | |
| beta chain (CD79B) | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.05; | |
| | | | | | | | | Adjusted P: | |
| | | | | | | | | 1.29E−02) | |
| | | | | | | | | mRNA (RNA-seq): | |
| | | | | | | | | lesional vs | |
| | | | | | | | | perilesional | |
| | | | | | | | | (P < 0.01) | |
| Collagen alpha-1(IV) | COL4A1 | P02462/ | COL4A1 | NA | Angiogenesis; | Lesional skin | Blood test, | Adjusted P: | FIG. 9F |
| chain (COL4A1) | | SEQ ID | | | Endothelial cell growth | sample and | tape strip, punch | Protein | |
| | | NO: 23 | | | regulation | peripheral | biopsies, microbiopsy | (blood proteomics): | |
| | | | | | | blood | and any type of | Correlation | |
| | | | | | | | surgical specimen | with dT count | |
| | | | | | | | | (Slope: 0.05; | |
| | | | | | | | | 2.54E−02) | |
| | | | | | | | | mRNA (RNA-seq): | |
| | | | | | | | | lesional vs | |
| | | | | | | | | perilesional | |
| | | | | | | | | (P < 0.01) | |
| C-type lectin domain | CLEC4D | Q8WX18/ | CLEC4D | Mpcl | T-cell/Th17 activation | Peripheral | Blood test | Protein | FIG. 9F |
| family 4 member D | | SEQ ID | | CD368 | | blood | | (blood proteomics): | |
| (CLEC4D) | | NO: 24 | | MCL | | | | Elevated vs. | |
| | | | | Dectin-3 | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.05; | |
| | | | | | | | | Adjusted P: | |
| | | | | | | | | 3.41E−02) | |
| von Willebrand | VWF | P04275/ | VWF | NA | Extracellular matrix | Peripheral | Blood test | Protein | FIG. 9G |
| factor (VWF) | | SEQ ID | | | structure | blood | | (blood proteomics): | |
| | | NO: 25 | | | | | | Correlation | |
| | | | | | | | | with dT count | |
| | | | | | | | | (Slope: 0.05; | |
| | | | | | | | | Adjusted P: | |
| | | | | | | | | 7.46E−03) | |
| Interleukin 5 | IL-5RA | Q01344/ | IL5RA | CDw125 | Eosinophil regulation | Peripheral | Blood test | Protein | FIG. 9G |
| receptor subunit | | SEQ ID | | CD125 | | blood | | (blood proteomics): | |
| alpha (ILSRA) | | NO: 26 | | | | | | Correlation | |
| | | | | | | | | with dT count | |
| | | | | | | | | (Slope: 0.05; | |
| | | | | | | | | Adjusted P: | |
| | | | | | | | | 3.14E−02) | |
| Granulocyte colony- | G-CSF | P09919/ | CSF3 | C17orf33, | Granulocyte and | Peripheral | Blood test | Protein | FIG. 9G |
| stimulating factor | | SEQ ID | | GCSF | macrophage activation | blood | | (blood proteomics): | |
| (CSF3) | | NO: 27 | | | (in the case of | | | Elevated vs. | |
| | | | | | granulocytes | | | healthy | |
| | | | | | stimulates survival, | | | controls | |
| | | | | | proliferation, | | | (FDR < 0.05); | |
| | | | | | differentiation, and | | | Correlation | |
| | | | | | function); binds to G- | | | with dT count | |
| | | | | | CSFR; is produced by | | | (Slope: 0.04; | |
| | | | | | macrophages, KCs | | | Adjusted P: | |
| | | | | | | | | 1.50E−03) | |
| Protransforming | TGF-alpha | P01135/ | TGFA | NA | Epithelial regeneration; | Peripheral | Blood test | Protein | FIG. 9N |
| growth factor alpha | | SEQ ID | | | Neurogenesis | blood | | (blood proteomics): | |
| (TGFA) | | NO: 28 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 3.80E−02) | |
| Interleukin-2 | IL-2RA | P01589/ | IL2RA | CD25 | T-cell activation | Lesional skin | Blood test, | Protein | FIG. 9H |
| receptor subunit | | SEQ ID | | | | sample and | tape strip, punch | (blood proteomics): | |
| alpha (IL2RA) | | NO: 29 | | | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 6.75E−04) | |
| | | | | | | | | mRNA (RNA-seq): | |
| | | | | | | | | lesional vs | |
| | | | | | | | | perilesional | |
| | | | | | | | | (P < 0.05) | |
| Inter-alpha-trypsin | ITI-HC3 | Q06033/ | ITIH3 | H3P | Hyaluronan transport | Peripheral | Blood test | Protein | FIG. 9H |
| inhibitor heavy chain | | SEQ ID | | | | blood | | (blood proteomics): | |
| H3 (ITIH3) | | NO: 30 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 7.93E−04) | |
| Immunoglobulin | IgA Fc | P24071/ | FCAR | CD89 | Antibody-dependent | Peripheral | Blood test | Protein | FIG. 9H |
| alpha Fc receptor | receptor | SEQ ID | | | cell-mediated | blood | | (blood proteomics): | |
| (FCAR) | | NO: 31 | | | cytotoxicity | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 2.54E−02) | |
| C-C motif chemokine | CCL23 | P55773/ | CCL23 | MIP3, MPIF1, | T-cell, neutrophil and | Peripheral | Blood test | Protein | FIG. 9N |
| 23 (CCL23) | | SEQ ID | | SCYA23 | monocyte activation | blood | | (blood proteomics): | |
| | | NO: 32 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 2.35E−02) | |
| Neuronal cell | Nr-CAM | Q92823/ | NRCAM | KIAA0343 | Neurogenesis | Lesional skin | Blood test, | Protein | FIG. 9I |
| adhesion molecule | | SEQ ID | | | | sample and | tape strip, punch | (blood proteomics): | |
| (NRCAM) | | NO: 33 | | | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 1.88E−02) | |
| | | | | | | | | mRNA (RNA-seq): | |
| | | | | | | | | lesional vs | |
| | | | | | | | | perilesional | |
| | | | | | | | | (P < 0.05) | |
| Resistin (RETN) | RETN | Q9HD89/ | RETN | FIZZ3 | Myeloid cell | Peripheral | Blood test | Protein | FIG. 9I |
| | | SEQ ID | | ADSF | chemotaxis; Hormone | blood | | (blood proteomics): | |
| | | NO: 34 | | RETN1 | response | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 9.02E−03) | |
| Serpin A11 | SERPINA11 | Q86U17/ | SERPINA11 | NA | Endopeptidase | Peripheral | Blood test | Protein | FIG. 9N |
| (SERPINA11) | | SEQ ID | | | inhibitor activity | blood | | (blood proteomics): | |
| | | NO: 35 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 7.78E−03) | |
| C-type lectin domain | CLEC4G | Q6UXB4/ | CLEC4G | UNQ431 | Cell adhesion | Peripheral | Blood test | Protein | FIG. 9O |
| family 4 member G | | SEQ ID | | LSECTIN | | blood | | (blood proteomics): | |
| (CLEC4G) | | NO: 36 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.04; | |
| | | | | | | | | Adjusted P: | |
| | | | | | | | | 9.24E−04) | |
| Macrophage colony- | CSF-1 | P09603/ | CSF1 | M-CSF | Regulation of | Peripheral | Blood test | Protein | FIG. 9O |
| stimulating factor 1 | | SEQ ID | | MCSF | hematopoietic | blood | | (blood proteomics): | |
| (CSF1) | | NO: 37 | | MGC31930 | precursor cells | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 1.69E−03) | |
| Hepatocyte growth | HGF | P14210/ | HGF | HPTA | Growth factor | Lesional skin | Blood test, | Protein | FIG. 9I |
| factor (HGF) | | SEQ ID | | | | sample and | tape strip, punch | (blood proteomics): | |
| | | NO: 38 | | | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 4.14E−02) | |
| | | | | | | | | mRNA (RNA-seq): | |
| | | | | | | | | lesional vs | |
| | | | | | | | | perilesional | |
| | | | | | | | | (P < 0.05) | |
| Protein disulfide | CRELD2 | Q6UXH1/ | CRELD2 | MGC11256 | Isomerase | Lesional skin | Blood test, | Protein | FIG. 9J |
| isomerase CRELD2 | | SEQ ID | | | | sample and | tape strip, punch | (blood proteomics): | |
| (CRELD2) | | NO: 39 | | | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 4.39E−02) | |
| | | | | | | | | mRNA (RNA-seq): | |
| | | | | | | | | lesional vs | |
| | | | | | | | | perilesional | |
| | | | | | | | | (P < 0.05) | |
| EGF-containing | EFEMP1 | Q12805/ | EFEMP1 | S1-5 | Growth factor; Cells | Peripheral | Blood test | Protein | FIG. 9O |
| fibulin-like | | SEQ ID | | FBLN3 | adhesion; | blood | | (blood proteomics): | |
| extracellular matrix | | NO: 40 | | MTLV | Neurogenesis | | | Elevated vs. | |
| protein 1 (EFEMP1) | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 2.04E−02) | |
| Tumor necrosis | TNF-RIII | P36941/ | LTBR | D12S370, | Lipid metabolism; | Peripheral | Blood test | Protein | FIG. 9O |
| factor receptor | | SEQ ID | | TNFCR, | Immune response; Cell | blood | | (blood proteomics): | |
| superfamily member | | NO: 41 | | TNFR3, | death | | | Elevated vs. | |
| 3 (LTBR) | | | | TNFRSF3 | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 4.21E−03) | |
| Nucleoside | NDK 3 | Q13232/ | NME3 | DR-nm23 | Granulocyte | Peripheral | Blood test | Protein | FIG. 9O |
| diphosphate kinase 3 | | SEQ ID | | NM23-H3 | regulation; Nucleotide | blood | | (blood proteomics): | |
| (NME3) | | NO: 42 | | NDPKC | metabolism | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 4.08E−03) | |
| Cytoskeleton- | CKAP4 | Q07065/ | CKAP4 | P63 | Mediates cell | Peripheral | Blood test | Protein | FIG. 9P |
| associated protein 4 | | SEQ ID | | CLIMP-63 | proliferation | blood | | (blood proteomics): | |
| (CKAP4) | | NO: 43 | | CLIMP63 | | | | Elevated vs. | |
| | | | | ERGIC-63 | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 4.07E−02) | |
| CD276 antigen | CD276 | Q5ZPR3/ | CD276 | B7H3 | T-cell/Th17 activation; | Lesional skin | Blood test, | Protein | FIG. 9J |
| (CD276) | | SEQ ID | | | NK cell regulation | sample and | tape strip, punch | (blood proteomics): | |
| | | NO: 44 | | | | peripheral | biopsies, microbiopsy | Elevated vs. | |
| | | | | | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 2.54E−02) | |
| | | | | | | | | mRNA (RNA-seq): | |
| | | | | | | | | lesional vs | |
| | | | | | | | | perilesional | |
| | | | | | | | | (P < 0.05) | |
| Spondin-2 (SPON2) | SPON2 | Q9BUD6/ | SPON2 | DIL1 | Cell adhesion; Innate | Peripheral | Blood test | Protein | FIG. 9P |
| | | SEQ ID | | | immunity | blood | | (blood proteomics): | |
| | | NO: 45 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 2.23E−02) | |
| Gamma-glutamyl | GGH | Q92820/ | GGH | GATD10 | Hydrolase; Neutrophil | Peripheral | Blood test | Protein | FIG. 9P |
| hydrolase (GGH) | | SEQ ID | | | degranulation | blood | | (blood proteomics): | |
| | | NO: 46 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 2.54E−02) | |
| Metalloproteinase | TIMP-1 | P01033/ | TIMP1 | CLGI, TIMP | Growth factor; | Peripheral | Blood test | Protein | FIG. 9J |
| inhibitor 1 (TIMP1) | | SEQ ID | | | Proteinase inhibitor | blood | | (blood proteomics): | |
| | | NO: 47 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 4.45E−02) | |
| T-lymphocyte | Ly-9 | Q9HBG7/ | LY9 | CD229 | Cell Adhesion | Lesional skin | Blood test, | Protein | FIG. 9K |
| surface antigen Ly-9 | | SEQ ID | | mLY9 | | sample and | tape strip, punch | (blood proteomics): | |
| (LY9) | | NO: 48 | | SLAMF3 | | peripheral | biopsies, microbiopsy | Elevated vs. | |
| | | | | hly9 | | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.03; | |
| | | | | | | | | Adjusted P: | |
| | | | | | | | | 4.93E−02) | |
| | | | | | | | | mRNA (RNA-seq): | |
| | | | | | | | | lesional vs | |
| | | | | | | | | perilesional | |
| | | | | | | | | (P < 0.05) | |
| Multiple coagulation | MCFD2 | Q8NI22/ | MCFD2 | F5F8D | Protein transport; | Peripheral | Blood test | Protein | FIG. 9P |
| factor deficiency | | SEQ ID | | LMAN1IP | Secretion of | blood | | (blood proteomics): | |
| protein 2 (MCFD2) | | NO: 49 | | SDNSF | coagulation factor | | | Correlation | |
| | | | | | | | | with dT count | |
| | | | | | | | | (Slope: 0.03; | |
| | | | | | | | | Adjusted P: | |
| | | | | | | | | 1.61E−02) | |
| Transcobalamin-2 | TC-2 | P20062/ | TCN2 | D22S676 | Vitamin B12 binding | Peripheral | Blood test | Protein | FIG. 9K |
| (TCN2) | | SEQ ID | | D22S750 | and transport protein | blood | | (blood proteomics): | |
| | | NO: 50 | | TC2 | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.02; | |
| | | | | | | | | Adjusted P: | |
| | | | | | | | | 3.83E−02) | |
| Glutaminyl-peptide | QPCT | Q16769/ | QPCT | QC | Peptide biosynthesis | Peripheral | Blood test | Protein | FIG. 9P |
| cyclotransferase | | SEQ ID | | GCT | | blood | | (blood proteomics): | |
| (QPCT) | | NO: 51 | | | | | | Elevated vs. | |
| | | | | | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.02; | |
| | | | | | | | | Adjusted P: | |
| | | | | | | | | 3.24E−02) | |
| Hypoxia up- | HYOU1 | Q9Y4L1/ | HYOU1 | ORP150 | Molecular chaperone: | Lesional skin | Blood test, | Protein | FIG. 9K |
| regulated protein 1 | | SEQ ID | | HSP12A | Cytoprotective in | sample and | tape strip, punch | (blood proteomics): | |
| (HYOU1) | | NO: 52 | | Grp170 | cellular oxygen | peripheral | biopsies, microbiopsy | Elevated vs. | |
| | | | | | depravation | blood | and any type of | healthy | |
| | | | | | | | surgical specimen | controls | |
| | | | | | | | | (FDR < 0.05); | |
| | | | | | | | | Correlation | |
| | | | | | | | | with dT count | |
| | | | | | | | | (Slope: 0.02; | |
| | | | | | | | | Adjusted P: | |
| | | | | | | | | 8.79E−03) | |
| | | | | | | | | mRNA (RNA-seq): | |
| | | | | | | | | lesional vs | |
| | | | | | | | | perilesional | |
| | | | | | | | | (P < 0.05) | |
| Tumor necrosis | BAFF | Q9Y275/ | TNFSF13B | AFF, BLYS, | B-cell activation; T- | Peripheral | Blood test | Protein | FIG. 9P |
| factor ligand | | SEQ ID | | TALL1, | cell/Th17 activation | blood | | (blood proteomics): | |
| superfamily member | | NO: 53 | | TNFSF20, | | | | Elevated vs. | |
| 13B (TNFSF13B) | | | | ZTNF4 | | | | healthy | |
| | | | | | | | | controls | |
| | | | | | | | | (FDR < 0.05); | |
| | | | | | | | | Correlation | |
| | | | | | | | | with dT count | |
| | | | | | | | | (Slope: 0.02; | |
| | | | | | | | | Adjusted P: | |
| | | | | | | | | 4.25E−02) | |
| C-C motif chemokine | CCL7 | P80098/ | CCL7 | MCP-3 | Chemoattractant; | Lesional skin | Tape strip, punch | mRNA (RNA-seq): | FIG. 9V |
| ligand 7 (CCL7) | | SEQ ID | | NC28 | Neutrophil | sample | biopsies, microbiopsy | lesional vs | |
| | | NO: 54 | | FIC | activation/degradation; | | and any type of | perilesional | |
| | | | | MARC | NK cell activation; acts | | surgical specimen | (3-fold) | |
| | | | | MCP3 | on monocytes, | | | | |
| | | | | | dendritic cells, | | | | |
| | | | | | neutrophils, NK cells, | | | | |
| | | | | | and activated T cells; | | | | |
| | | | | | binds to CCR2; is | | | | |
| | | | | | produced by | | | | |
| | | | | | leukocytes, KCs | | | | |
| C-X-C motif | CXC13 | O43927/ | CXCL13 | BLC | B-cell chemoattractant | Lesional skin | Tape strip, punch | mRNA (RNA-seq): | FIG. 9V |
| chemokine 13 | | SEQ ID | | BCA-1 | | sample | biopsies, microbiopsy | lesional vs | |
| (CXCL13) | | NO: 55 | | BLR1L | | | and any type of | perilesional | |
| | | | | ANGIE | | | surgical specimen | (P < 0.01) | |
| | | | | ANGIE2 | | | | | |
| Protocadherin-1 | PCDH1 | Q08174/ | PCDH1 | pc42 | Cell-cell interaction and | Peripheral | Blood test | Protein | FIG. 9Q |
| (PCDH1) | | SEQ ID | | | adhesion | blood | | (blood proteomics): | |
| | | NO: 56 | | | | | | Correlation | |
| | | | | | | | | with dT count | |
| | | | | | | | | (Slope: −0.01; | |
| | | | | | | | | Adjusted P: | |
| | | | | | | | | 6.75E−04) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| Brother of CDO | BOC | Q9BWV1/ | BOC | CDON2 | Cell adhesion | lesional skin | Blood test, | Protein | FIG. 9Q |
| (BOC) | | SEQ ID | | Boi | | sample and | tape strip, punch | (blood proteomics): | |
| | | NO: 57 | | | | peripheral | biopsies, microbiopsy | Correlation | |
| | | | | | | blood | and any type of | with dT count | |
| | | | | | | | surgical specimen | (Slope: −0.02; | |
| | | | | | | | | Adjusted P: | |
| | | | | | | | | 3.00E−02) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| | | | | | | | | mRNA (RNA-seq): | |
| | | | | | | | | lesional vs | |
| | | | | | | | | perilesional | |
| | | | | | | | | (P < 0.05) | |
| Matrix extracellular | MEPE | Q9NQ76/ | MEPE | NA | Bone mineralization; | Peripheral | Blood test | Protein | FIG. 9Q |
| phosphoglycoprotein | | SEQ ID | | | Cell proliferation and | blood | | (blood proteomics): | |
| (MEPE) | | NO: 58 | | | differentiation | | | Correlation | |
| | | | | | | | | with dT count | |
| | | | | | | | | (Slope: −0.03; | |
| | | | | | | | | Adjusted P: | |
| | | | | | | | | 1.79E−02) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| Disintegrin and | ADAM 23 | O75077/ | ADAM23 | MDC3 | Cell-cell interaction and | Peripheral | Blood test | Protein | FIG. 9Q |
| metalloproteinase | | SEQ ID | | | adhesion | blood | | (blood proteomics): | |
| domain-containing | | NO: 59 | | | | | | Correlation | |
| protein 23 | | | | | | | | with dT count | |
| (ADAM23) | | | | | | | | (Slope: −0.03; | |
| | | | | | | | | Adjusted P: | |
| | | | | | | | | 4.16E−02) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| Thimet | THOP1 | P52888/ | THOP1 | NA | Metabolism of | Peripheral | Blood test | Protein | FIG. 9Q |
| oligopeptidase | | SEQ ID | | | neuropeptides | blood | | (blood proteomics): | |
| (THOP1) | | NO: 60 | | | | | | Correlation | |
| | | | | | | | | with dT count | |
| | | | | | | | | (Slope: −0.03; | |
| | | | | | | | | Adjusted P: | |
| | | | | | | | | 7.17E−03) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| Interleukin-1 | IL-1RL2 | Q9HB29/ | IL1RL2 | IL1R-rp2 | T-cell activation | Peripheral | Blood test | Protein | FIG. 9Q |
| receptor-like 2 | | SEQ ID | | IL1RRP2 | | blood | | (blood proteomics): | |
| (IL1RL2) | | NO: 61 | | IL-36R | | | | Correlation | |
| | | | | | | | | with dT count | |
| | | | | | | | | (Slope: −0.03; | |
| | | | | | | | | Adjusted P: | |
| | | | | | | | | 4.39E−02) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| REST corepressor 1 | RCOR1 | Q9UKL0/ | RCOR1 | COREST | Transcription | Peripheral | Blood test | Protein | FIG. 9R |
| (RCOR1) | | SEQ ID | | KIAA0071 | regulation | blood | | (blood proteomics): | |
| | | NO: 62 | | | | | | Correlation | |
| | | | | | | | | with dT count | |
| | | | | | | | | (Slope: −0.03; | |
| | | | | | | | | Adjusted P: | |
| | | | | | | | | 4.93E−02) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| Tumor necrosis | EDAR | Q9UNE0/ | EDAR | ED5 | Mediates the | Peripheral | Blood test | Protein | FIG. 9R |
| factor receptor | | SEQ ID | | EDA3 | activation of NF-kappa- | blood | | (blood proteomics): | |
| superfamily member | | NO: 63 | | ED1R | B | | | Correlation | |
| EDAR (EDAR) | | | | EDA1R | | | | with dT count | |
| | | | | | | | | (Slope: −0.07; | |
| | | | | | | | | Adjusted P: | |
| | | | | | | | | 1.43E−02) | |
| | | | | | | | | (decreased | |
| | | | | | | | | level of | |
| | | | | | | | | protein | |
| | | | | | | | | associated | |
| | | | | | | | | with higher | |
| | | | | | | | | tunnel counts) | |
| C-C motif chemokine | CCL2 | P13500/ | CCL2 | MCP1 | Chemoattractant; acts | Lesional skin | Tape strip, punch | mRNA (RNA-seq): | FIG. 9U |
| 2 (CCL2) | | SEQ ID | | MCP-1 | on monocytes, | sample | biopsies, microbiopsy | lesional vs | |
| | | NO: 64 | | MCAF | memory T cells, | | and any type of | perilesional | |
| | | | | SMC-CF | dendritic cells; binds to | | surgical specimen | (P < 0.05) | |
| | | | | GDCF-2 | CCR2 and CCR4; T-cell | | | Protein: | |
| | | | | HC11 | activation; Immune cell | | | lesional vs | |
| | | | | MGC9434 | extravasation; is | | | perilesional | |
| | | | | | produced by monocytes, | | | (P < 0.05) | |
| | | | | | macrophages, dendritic | | | | |
| | | | | | cells, KCs | | | | |
| C-C motif chemokine | CCL3 | P10147/ | CCL3 | GOS19-1 | Chemoattractant; | Lesional skin | Tape strip, punch | mRNA (RNA-seq): | FIG. 9U |
| 3 (CCL3) | | SEQ ID | | LD78ALPHA | Neutrophil activation; | sample | biopsies, microbiopsy | lesional vs | |
| | | NO: 65 | | MIP-1-alpha | binds CCR1, 4, 5; | | and any type of | perilesional | |
| | | | | LD78 | produced by | | surgical specimen | (P < 0.05) | |
| | | | | SCI | leukocytes | | | | |
| C-C motif chemokine | CCL4 | P13236/ | CCL4 | MIP-1-beta | Chemoattractant; NK | Lesional skin | Tape strip, punch | mRNA (RNA-seq): | FIG. 9S |
| 4 (CCL4) | | SEQ ID | | Act-2 | cell activation; Immune | sample | biopsies, microbiopsy | lesional vs | |
| | | NO: 66 | | AT744.1 | cell extravasation: | | and any type of | perilesional | |
| | | | | | Angioenesis; acts on | | surgical specimen | (P < 0.05) | |
| | | | | | NK cells, monocytes, | | | Protein: | |
| | | | | | other leukocytes; binds | | | lesional vs | |
| | | | | | to CCR5, 8; is produced | | | perilesional | |
| | | | | | by monocytes, T cells, | | | (P < 0.01) | |
| | | | | | dendritic cells, | | | | |
| | | | | | neutrophils, NK cells | | | | |
| C-C motif chemokine | CCL5 | P13501/ | CCL5 | RANTES | Chemoattractant; T- | Lesional skin | Tape strip, punch | mRNA (RNA-seq): | FIG. 9S |
| ligand 5 (CCL5) | | SEQ ID | | SISd | cell activation; NK cell | sample | biopsies, microbiopsy | lesional vs | |
| | | NO: 67 | | TCP228 | activation; Immune cell | | and any type of | perilesional | |
| | | | | MGC17164 | extravasation; binds to | | surgical specimen | (P < 0.05) | |
| | | | | | CCR5; is produced by T | | | Protein: | |
| | | | | | cells, monocytes, KCs | | | lesional vs | |
| | | | | | | | | perilesional | |
| | | | | | | | | (P < 0.05); | |
| C-C motif chemokine | CCL20 | P78556/ | CCL20 | LARC | Chemoattractant; T- | Lesional skin | Blood test, | Protein | FIG. 9S |
| 20 (CCL20) | | SEQ ID | | MIP-3a | cell/Th17 activation; | sample and | tape strip, punch | (blood proteomics): | |
| | | NO: 68 | | exodus-1 | acts on B cells and DCs; | peripheral | biopsies, microbiopsy | Elevated vs. | |
| | | | | ST38 | binds to CCR6; is | blood | and any type of | healthy | |
| | | | | CKb4 | produced by | | surgical specimen | controls | |
| | | | | | neutrophils, NK cells, | | | (FDR < 0.05); | |
| | | | | | Th17 cells, B cells, DCs, | | | Protein: | |
| | | | | | LCs, macrophages, and | | | lesional vs | |
| | | | | | KCs | | | perilesional | |
| | | | | | | | | (P < 0.001) | |
| Growth-regulated | CXCL1 | P09341/ | CXCL1 | SCYB1 | Chemoattractant; | Lesional skin | Tape strip, punch | mRNA (RNA-seq): | FIG. 9T |
| alpha protein | | SEQ ID | | GROa | Neutrophil | sample | biopsies, microbiopsy | lesional vs | |
| (CXCL1) | | NO: 69 | | MGSA-a | activation/degradation; | | and any type of | perilesional | |
| | | | | NAP-3 | binds to CXCR2; is | | surgical specimen | (P < 0.05); | |
| | | | | | produced by | | | Protein: | |
| | | | | | macrophages, | | | lesional vs | |
| | | | | | neutrophils, Th17 cells, | | | perilesional | |
| | | | | | and KCs | | | (P < 0.01) | |
| C-X-C motif | CXCL8 | P10145/ | CXCL8 | SCYB8 | Chemoattractant; | Lesional skin | Tape strip, punch | mRNA (RNA-seq): | FIG. 9T |
| chemokine 8 (CXCL8) | | SEQ ID | | LUCT | Neutrophil | sample | biopsies, microbiopsy | lesional vs | |
| | | NO: 70 | | LECT | activation/degradation; | | and any type of | perilesional | |
| | | | | MDNCF | binds to CXCR1 > CXCR2; is | | surgical specimen | (P < 0.01); | |
| | | | | TSG-1 | produced by macrophages, KCs | | | Protein: | |
| | | | | IL-8 | | | | lesional vs | |
| | | | | MONAP | | | | perilesional | |
| | | | | K60 | | | | (P < 0.01) | |
| | | | | GCP1 | | | | | |
| | | | | NAP1 | | | | | |
| Platelet-derived | PDGF-AA | P04085/ | PDGFA | PDGF1 | Growth Factor; Cell | Lesional skin | Tape strip, punch | Protein: | FIG. 9T |
| growth factor | | SEQ ID | | PDGF-A | proliferation, migration | sample | biopsies, microbiopsy | lesional vs | |
| subunit A (PDFGA) | | NO: 71 | | | and chemotaxis; acts | | and any type of | perilesional | |
| | | | | | on fibroblasts; binds to | | surgical specimen | (P < 0.05) | |
| | | | | | PDGF-Ra and b; is | | | | |
| | | | | | produced e.g. by | | | | |
| | | | | | macrophages | | | | |
| C-X-C chemokine | CXCR2 | P25025/ | CXCR2 | CMKAR2 | Receptor for GRO- | Lesional skin | Tape strip, punch | mRNA (RNA-seq): | FIG. 10 |
| receptor type 2 | | SEQ ID | | CD182 | alpha and CXCL8 | sample | biopsies, microbiopsy | lesional vs | |
| (CXCR2) | | NO: 72 | | | | | and any type of | perilesional | |
| | | | | | | | surgical specimen | (2-fold) | |
| C-C chemokine | CCR6 | P51684/ | CCR6 | CKR-L3 | Receptor for CCL20 | Lesional skin | Tape strip, punch | mRNA (RNA-seg): | FIG. 10 |
| receptor type 6 | | SEQ ID | | GPR-CY4 | | sample | biopsies, microbiopsy | lesional vs | |
| (CCR6) | | NO: 73 | | CMKBR6 | | | and any type of | perilesional | |
| | | | | GPR29 | | | surgical specimen | (3-fold) | |
| | | | | DRY-6 | | | | | |
| | | | | DCR2 | | | | | |
| | | | | BN-1 | | | | | |
| | | | | CD196 | | | | | |

As further shown in Table 1, the above research and tests resulted in the identification of a significant elevation in levels of biomarkers in the peripheral blood of HS patients compared to Healthy Controls (HCs). These number and variety of biomarkers reflect the remarkable inflammatory activity and complexity of HS. Here, a paired t-test was employed, setting the significance cutoff at FDR<0.05.

Regression analysis was also conducted to establish a correlation between biomarker levels and the number of draining tunnels. The slope value in Table 1 corresponds to the coefficient value; the higher the absolute value the stronger the correlation is. Notably, high draining tunnel counts were linked with high levels of 58 biomarkers. High draining tunnel counts also correlated with decreasing levels of 8 other biomarkers: PCDH1, BOC, MEPE, ADAM23, THOP1, IL1RL2, RCOR1, and EDAR. These findings underscore the active role of draining tunnels in HS pathophysiology, positioning them as key contributors to HS pathology.

The molecular signature of the draining tunnel biomarkers in the peripheral blood, together with additional HS-mediator, -pathway and -receptor biomarkers were explored in tissue from HS draining tunnels and compared with perilesional tissue and inflammatory nodules at the mRNA level and/or protein level using RNA-seq and/or cytokine array or ELISA. In the HS skin tissue, higher expression of biomarkers was observed in draining tunnels compared to per-lesional skin or inflammatory nodules.

The identification of such a panel of biomarkers having functional relevance for not only timely diagnosis of HS, but also (i) assessment of inflammatory disease activity such as tunnel count correlation, (ii) selection and initiation of appropriate therapy, and (iii) monitoring treatment response and managing long-term therapy of HS was unexpected.

### Example 3

Elafin and IL-19 (Circulating Biomarkers) Levels Normalized after Treatment to Those Found in a Healthy Control Population.

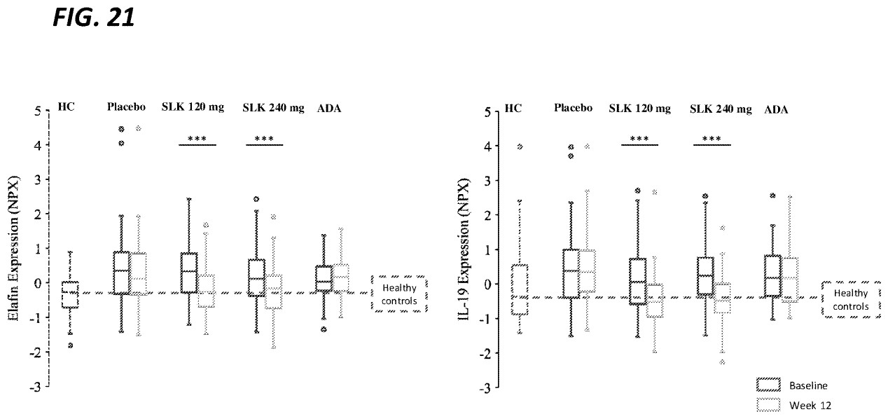

After 12 weeks of treatment with the IL-17 inhibitor SLK, the levels of two circulating biomarkers related to HS activity were normalized to those found in a healthy control population (n=50). The results indicate molecular healing of HS patients and confirm the value of these biomarkers in HS diagnosis and monitoring. See FIG. 21 and Table 2.

TABLE 2
Biomarkers for Monitoring Molecular
Response to IL-17 Inhibitor Treatment

| Biomarkers | Data/Technical Effect |
|---|---|
| Elafin (PI3) | FIG. 21 |
| Interleukin-19 (IL19) | |

### Example 4

Biomarkers Identifying HS super-responders.

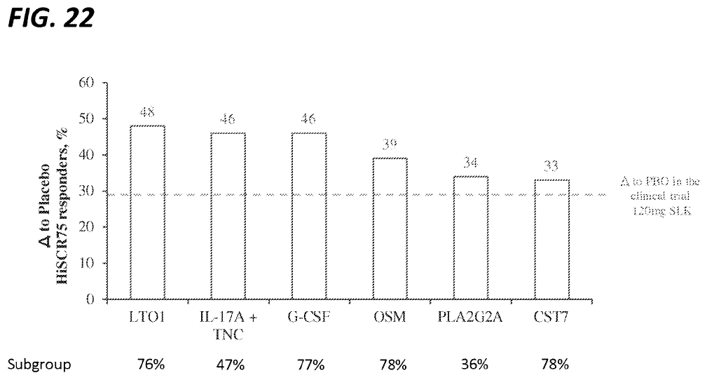

The ‘Subgroup Identification based on Differential Effect Search’ (SIDES) analysis (Lipkovich et al., Stat Med. 30(21): 2601-21 (2011)) was employed as a partitioning method to determine treatment responses within specific patient groups. Subgroups of patients with higher biomarker expression levels demonstrated an enhanced clinical outcome in response to 120 mg sonelokimab, as evidenced by the delta to placebo in HiSCR75 response compared to unselected patients. Applied threshold (NPX): LT01>−0.4123; IL-17A>−0.8321; TNC>−0.0153; CSF3>−0.2455; OSM>−0.4974; PLA2G2A>0.6373; CST7>−0.628. In this way, each of a) LOT1, b) a combination of IL17A and TNC, c) G-CSF, d) OSM, and e) PLA2G2A were surprisingly revealed as capable of identifying HS super-responders to an IL-17 inhibitor. See FIG. 22 and Table 3.

Many of the biomarkers described in Table 1 reflect pathways involved in HS pathophysiology such as neutrophil activation and degradation, angiogenesis and neurogenesis, innate immunity, T cell and especially Th17 cell activation, B cell activation, epithelial regeneration, extracellular matrix organization, keratinocyte activation, immune cell extravasation, and N K and CD8+ cell activation. However, other biomarkers have not been implicated in inflammatory conditions including HS. For example, LOT1 (also termed PLAGL1 or ZAC1; see also Table 3) is a zinc-finger nuclear transcription factor known to play a role in regulating cell growth. Associated diseases include growth retardation, neonatal diabetes mellitus and various types of cancers (Abdollahi, J Cell Physiol. 2007 January; 210(1):16-25), but a role for this biomarker in inflammatory diseases including HS has not been previously described. Additionally, CST7, COL4A1, NRCAM, HYOU1, SDC1 have not been previously described as having a role in HS.

TABLE 3
Biomarkers Indicative of Superresponders (Patient Population Highly
Responsive to Treatment) with an IL-17A +F-Specific Nanobody

| | Data/Technical |
|---|---|
| Biomarkers | Effect |
| Protein LTO1 homolog (LTO1) | FIG. 22 |
| Interleukin-17A (IL17A)+ Tenascin C (TNC) | |
| Granulocyte colony-stimulating factor (CSF3) | |
| Oncostatin-M (OSM) | |
| Phospholipase A2, membrane associated (PLA2G2A) | |
| Cystatin-F (CST7) | |

1. A method of treating hidradenitis suppurativa (HS), comprising administering a medicament comprising an IL-17A- and/or IL-17F-inhibitor to a subject, wherein the subject has been identified as having an elevated level of one or more biomarkers selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

2. The method of claim 1, wherein the subject has a phenotype that includes one or more draining tunnels in the skin.

3. The method of claim 1, wherein the more elevated the level of the one or more biomarkers, the higher the number of draining tunnels are present in the subject.

4. The method of claim 1, wherein as assessed by clinical inspection the subject has no draining tunnels.

5. The method of claim 1, wherein the subject has been clinically diagnosed as having HS in Hurley Stage I or II or III.

6. The method of claim 1, wherein the subject has been clinically diagnosed as having mild HS, moderate HS, moderate-to-severe HS, severe HS, or juvenile HS.

7. The method of claim 1, wherein the IL-17A- and/or IL-17F-inhibitor comprises a nanobody.

8. The method of claim 7, wherein the nanobody is sonelokimab (SLK).

9. The method of claim 1, wherein the level is elevated as compared to: (i) the level present in non-lesional skin, or (ii) the level present in peripheral blood of a healthy patient.

10. The method of claim 1, wherein the level is elevated as compared to a reference value and the reference value is (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue.

11. The method of claim 1, which comprises assaying a tissue sample or body fluid from the subject for a level of one or more of the biomarkers prior to treating.

12. The method of claim 1, wherein the biomarkers comprise protein and/or mRNA biomarkers.

13. The method of claim 1, wherein the one or more biomarkers are selected from IL6, PLA2G2A, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13.

14. The method of claim 1, wherein the one or more biomarkers are selected from IL6, PLA2G2A, IL19, PI3, CST7, IL17A, IL17F, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, and TNSFSF13B.

15. The method of claim 1, wherein the one or more biomarkers are selected from IL6, PLA2G2A, IL19, PI3, CST7, GH1, MZB1, IL1B, IFNG, TNC, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, PDFGA, CXCR2, CCR6, and CXCL13.

16. A method of treating hidradenitis suppurativa (HS), comprising administering a medicament comprising an IL-17A- and/or IL-17F-inhibitor to a subject, wherein the subject has been identified as having a reduced level of one or more biomarkers selected from PCDH1, BOC, MEPE, ADAM23, THOP1, IL1RL2, RCOR1, and EDAR.

17. The method of claim 16, wherein the subject has a phenotype that includes one or more draining tunnels in the skin.

18. The method of claim 16, wherein the more reduced the level of the one or more biomarkers, the higher the number of draining tunnels are present in the subject.

19. The method of claim 16, wherein as assessed by clinical inspection the subject has no draining tunnels.

20. The method of claim 16, wherein the subject has been clinically diagnosed as having HS in Hurley Stage I or II or III.

21. The method of claim 16, wherein the subject has been clinically diagnosed as having mild HS, moderate HS, moderate-to-severe HS, severe HS, or juvenile HS.

22. The method of claim 16, wherein the IL-17A- and/or IL-17F-inhibitor comprises a nanobody.

23. The method of claim 22, wherein the nanobody is sonelokimab (SLK).

24. The method of claim 16, wherein the level is reduced as compared to: (i) the level present in non-lesional skin, or (ii) the level present in peripheral blood of a healthy patient.

25. The method of claim 16, wherein the level is reduced as compared to a reference value and the reference value is (i) the biomarker's expression level from the corresponding body fluid or tissue sample obtained from a healthy subject; (ii) the average level of the biomarker expressed in the corresponding body fluid or tissue of a plurality of healthy subjects; or (iii) the average level of the biomarker expressed in healthy tissue.

26. The method of claim 16, which comprises assaying a tissue sample or body fluid from the subject for a level of one or more of the biomarkers prior to treating.

27. The method of claim 16, wherein the biomarkers comprise protein and/or mRNA biomarkers.

28. A method of detecting hidradenitis suppurativa (HS), which comprises assaying a sample from a subject having or at risk of having HS for elevated levels of one or more of IL6, PLA2G2A, IL19, PI3, CST7, IL17A, IL17F, GH1, MZB1, IL1B, IFNG, TN C, CXCL9, SLAMF7, VEGFA, IL17C, SLAMF1, SDC1, OSM, LBP, REG3A, CD79B, COL4A1, CLEC4D, VWF, IL5RA, CSF3, TGFA, IL2RA, ITIH3, FCAR, CCL23, NRCAM, RETN, SERPINA11, CLEC4G, CSF1, HGF, CRELD2, EFEMP1, LTBR, NME3, CKAP4, CD276, SPON2, GGH, TIMP1, LY9, MCFD2, TCN2, QPCT, HYOU1, TNSFSF13B, CCL2, CCL3, CCL4, CCL5, CCL7, CCL20, CXCL1, CXCL8, PDFGA, CXCR2, CCR6, and CXCL13; or for reduced levels of one or more of PCDH1, BOC, MEPE, ADAM23, THOP1, IL1RL2, RCOR1, and EDAR.


SEQUENCE LISTING (73 sequences)

SEQ ID NO: 1 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 144 aa, Homo sapiens
MKTLLLLAVIMIFGLLQAHGNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

SEQ ID NO: 3 - protein, 177 aa, Homo sapiens
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 4 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 5 - protein, 145 aa, Homo sapiens
MRAAGTLLAFCCLVLSTTGGPSPDTCSQDLNSRVKPGFPKTIKTNDPGVLQAARYSVEKFNNCTNDMFLFKESRITRALVQIVKGLKYMLEVEIGRTTCKKNQHLRLDDCDFQTNHTLKQTLSCYSEVWVVPWLQHFEVPVLRCH

SEQ ID NO: 6 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 7 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 8 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 9 - protein, 189 aa, Homo sapiens
MRLSLPLLLLLLGAWAIPGGLGDRAPLTATAPQLDDEEMYSAHMPAHLRCDACRAVAYQMWQNLAKAETKLHTSNSGGRRELSELVYTDVLDRSCSRNWQDYGVREVDQVKRLTGPGLSEGPEPSISVMVTGGPWPTRLSRTCLHYLGEFGEDQIYEAHQQGRGALEALLCGGPQGACSEKVSATREEL

SEQ ID NO: 10 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 11 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 12 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 13 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 14 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 15 - protein, 395 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 16 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 17 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 18 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 19 - protein, 252 aa, Homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 20 - protein, 481 aa, Homo sapiens
MGALARALPSILLALLLTSTPEALGANPGLVARITDKGLQYAAQEGLLALQSELLRITLPDFTGDLRIPHVGRGRYEFHSLNIHSCELLHSALRPVPGQGLSLSISDSSIRVQGRWKVRKSFFKLQGSFDVSVKGISISVNLLLGSESSGRPTVTASSCSSDIADVEVDMSGDLGWLLNLFHNQIESKFQKVLESRICEMIQKSVSSDLQPYLQTLPVTTEIDSFADIDYSLVEAPRATAQMLEVMFKGEIFHRNHRSPVTLLAAVMSLPEEHNKMVYFAISDYVFNTASLVYHEEGYLNFSITDDMIPPDSNIRLTTKSFRPFVPRLARLYPNMNLELQGSVPSAPLLNFSPGNLSVDPYMEIDAFVLLPSSSKEPVFRLSVATNVSATLTFNTSKITGFLKPGKVKVELKESKVGLFNAELLEALLNYYILNTFYPKFNDKLAEGFPLPLLKRVQLYDLGLQIHKDFLFLGANVQYMRV

SEQ ID NO: 21 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 22 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 23 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 24 - protein, 215 aa, Homo sapiens
MGLEKPQSKLEGGMHPQLIPSVIAVVFILLLSVCFIASCLVTHHNFSRCKRGTGVHKLEHHAKLKCIKEKSELKSAEGSTWNCCPIDWRAFQSNCYFPLTDNKTWAESERNCSGMGAHLMTISTEAEQNFIIQFLDRRLSYFLGLRDENAKGQWRWVDQTPFNPRRVFWHKNEPDNSQGENCVVLVYNQDKWAWNDVPCNFEASRICKIPGTTLN

SEQ ID NO: 25 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 26 - protein, 420 aa, Homo sapiens
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 27 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 28 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 29 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 30 - protein, 890 aa, Homo sapiens
MAFAWWPCLILALLSSLAASGFPRSPFRLLGKRSLPEGVANGIEVYSTKINSKVTSRFAHNVVTMRAVNRADTAKEVSFDVELPKTAFITNFTLTIDGVTYPGNVKEKEVAKKQYEKAVSQGKTAGLVKASGRKLEKFTVSVNVAAGSKVTFELTYEELLKRHKGKYEMYLKVQPKQLVKHFEIEVDIFEPQGISMLDAEASFITNDLLGSALTKSFSGKKGHVSFKPSLDQQRSCPTCTDSLLNGDFTITYDVNRESPGNVQIVNGYFVHFFAPQGLPVVPKNVAFVIDISGSMAGRKLEQTKEALLRILEDMQEEDYLNFILFSGDVSTWKEHLVQATPENLQEARTFVKSMEDKGMTNINDGLLRGISMLNKAREEHRIPERSTSIVIMLTDGDANVGESRPEKIQENVRNAIGGKFPLYNLGFGNNLNYNFLENMALENHGFARRIYEDSDADLQLQGFYEEVANPLLTGVEMEYPENAILDLTQNTYQHFYDGSEIVVAGRLVDEDMNSFKADVKGHGATNDLTFTEEVDMKEMEKALQERDYIFGNYIERLWAYLTIEQLLEKRKNAHGEEKENLTARALDLSLKYHFVTPLTSMVVTKPEDNEDERAIADKPGEDAEATPVSPAMSYLTSYQPPQNPYYYVDGDPHFIIQIPEKDDALCFNIDEAPGTVLRLIQDAVTGLTVNGQITGDKRGSPDSKTRKTYFGKLGIANAQMDFQVEVTTEKITLWNRAVPSTFSWLDTVTVTQDGLSMMINRKNMVVSFGDGVTFVVVLHQVWKKHPVHRDFLGFYVVDSHRMSAQTHGLLGQFFQPFDFKVSDIRPGSDPTKPDATLVVKNHQLIVTRGSQKDYRKDASIGTKVVCWFVHNNGEGLIDGVHTDYIVPNLF

SEQ ID NO: 31 - protein, 287 aa, Homo sapiens
MDPKQTTLLCLVLCLGQRIQAQEGDFPMPFISAKSSPVIPLDGSVKIQCQAIREAYLTQLMIIKNSTYREIGRRLKFWNETDPEFVIDHMDANKAGRYQCQYRIGHYRFRYSDTLELVVTGLYGKPFLSADRGLVLMPGENISLTCSSAHIPFDRFSLAKEGELSLPQHQSGEHPANFSLGPVDLNVSGIYRCYGWYNRSPYLWSFPSNALELVVTDSIHQDYTTQNLIRMAVAGLVLVALLAILVENWHSHTALNKEASADVAEPSWSQQMCQPGLTFARTPSVCK

SEQ ID NO: 32 - protein, 120 aa, Homo sapiens
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 33 - protein, 1304 aa, Homo sapiens
MQLKIMPKKKRLSAGRVPLILFLCQMISALEVPLDPKLLEDLVQPPTITQQSPKDYIIDPRENIVIQCEAKGKPPPSFSWTRNGTHFDIDKDPLVTMKPGTGTLIINIMSEGKAETYEGVYQCTARNERGAAVSNNIVVRPSRSPLWTKEKLEPITLQSGQSLVLPCRPPIGLPPPIIFWMDNSFQRLPQSERVSQGLNGDLYFSNVLPEDTREDYICYARFNHTQTIQQKQPISVKVISVDELNDTIAANLSDTEFYGAKSSRERPPTFLTPEGNASNKEELRGNVLSLECIAEGLPTPIIYWAKEDGMLPKNRTVYKNFEKTLQIIHVSEADSGNYQCIAKNALGAIHHTISVRVKAAPYWITAPQNLVLSPGEDGTLICRANGNPKPRISWLTNGVPIEIAPDDPSRKIDGDTIIFSNVQERSSAVYQCNASNEYGYLLANAFVNVLAEPPRILTPANTLYQVIANRPALLDCAFFGSPLPTIEWFKGAKGSALHEDIYVLHENGTLEIPVAQKDSTGTYTCVARNKLGMAKNEVHLEIKDPTWIVKQPEYAVVQRGSMVSFECKVKHDHTLSLTVLWLKDNRELPSDERFTVDKDHLVVADVSDDDSGTYTCVANTTLDSVSASAVLSVVAPTPTPAPVYDVPNPPFDLELTDQLDKSVQLSWTPGDDNNSPITKFIIEYEDAMHKPGLWHHQTEVSGTQTTAQLKLSPYVNYSFRVMAVNSIGKSLPSEASEQYLTKASEPDKNPTAVEGLGSEPDNLVITWKPLNGFESNGPGLQYKVSWRQKDGDDEWTSVVVANVSKYIVSGTPTFVPYLIKVQALNDMGFAPEPAVVMGHSGEDLPMVAPGNVRVNVVNSTLAEVHWDPVPLKSIRGHLQGYRIYYWKTQSSSKRNRRHIEKKILTFQGSKTHGMLPGLEPFSHYTLNVRVVNGKGEGPASPDRVFNTPEGVPSAPSSLKIVNPTLDSLTLEWDPPSHPNGILTEYTLKYQPINSTHELGPLVDLKIPANKTRWTLKNLNFSTRYKFYFYAQTSAGSGSQITEEAVTTVDEAGILPPDVGAGKVQAVNPRISNLTAAAAETYANISWEYEGPEHVNFYVEYGVAGSKEEWRKEIVNGSRSFFGLKGLMPGTAYKVRVGAVGDSGFVSSEDVFETGPAMASRQVDIATQGWFIGLMCAVALLILILLIVCFIRRNKGGKYPVKEKEDAHADPEIQPMKEDDGTFGEYSDAEDHKPLKKGSRTPSDRTVKKEDSDDSLVDYGEGVNGQFNEDGSFIGQYSGKKEKEPAEGNESSEAPSPVNAMNSFV

SEQ ID NO: 34 - protein, 108 aa, Homo sapiens
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 35 - protein, 422 aa, Homo sapiens
MGPAWLWLLGTGILASVHCQPLLAHGDKSLQGPQPPRHQLSEPAPAYHRITPTITNFALRLYKELAADAPGNIFFSPVSISTTLALLSLGAQANTSALILEGLGFNLTETPEADIHQGFRSLLHTLALPSPKLELKVGNSLFLDKRLKPRQHYLDSIKELYGAFAFSANFTDSVTTGRQINDYLRRQTYGQVVDCLPEFSQDTFMVLANYIFFKAKWKHPFSRYQTQKQESFFVDERTSLQVPMMHQKEMHRFLYDQDLACTVLQIEYRGNALALLVLPDPGKMKQVEAALQPQTLRKWGQLLLPSLLDLHLPRFSISGTYNLEDILPQIGLTNILNLEADFSGVTGQLNKTISKVSHKAMVDMSEKGTEAGAASGLLSQPPSLNTMSDPHAHFNRPFLLLLWEVTTQSLLFLGKVVNPVAG

SEQ ID NO: 36 - protein, 293 aa, Homo sapiens
MDTTRYSKWGGSSEEVPGGPWGRWVHWSRRPLFLALAVLVTTVLWAVILSILLSKASTERAALLDGHDLLRTNASKQTAALGALKEEVGDCHSCCSGTQAQLQTTRAELGEAQAKLMEQESALRELRERVTQGLAEAGRGREDVRTELFRALEAVRLQNNSCEPCPTSWLSFEGSCYFFSVPKTTWAAAQDHCADASAHLVIVGGLDEQGFLTRNTRGRGYWLGLRAVRHLGKVQGYQWVDGVSLSFSHWNQGEPNDAWGRENCVMMLHTGLWNDAPCDSEKDGWICEKRHNC

SEQ ID NO: 37 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 38 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 39 - protein, 353 aa, Homo sapiens
MRLPRRAALGLLPLLLLLPPAPEAAKKPTPCHRCRGLVDKFNQGMVDTAKKNFGGGNTAWEEKTLSKYESSEIRLLEILEGLCESSDFECNQMLEAQEEHLEAWWLQLKSEYPDLFEWFCVKTLKVCCSPGTYGPDCLACQGGSQRPCSGNGHCSGDGSRQGDGSCRCHMGYQGPLCTDCMDGYFSSLRNETHSICTACDESCKTCSGLTNRDCGECEVGWVLDEGACVDVDECAAEPPPCSAAQFCKNANGSYTCEECDSSCVGCTGEGPGNCKECISGYAREHGQCADVDECSLAEKTCVRKNENCYNTPGSYVCVCPDGFEETEDACVPPAEAEATEGESPTQLPSREDL

SEQ ID NO: 40 - protein, 493 aa, Homo sapiens
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 41 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 42 - protein, 169 aa, Homo sapiens
MICLVLTIFANLFPAACTGAHERTFLAVKPDGVQRRLVGEIVRRFERKGFKLVALKLVQASEELLREHYAELRERPFYGRLVKYMASGPVVAMVWQGLDVVRTSRALIGATNPADAPPGTIRGDFCIEVGKNLIHGSDSVESARREIALWFRADELLCWEDSAGHWLYE

SEQ ID NO: 43 - protein, 602 aa, Homo sapiens
MPSAKQRGSKGGHGAASPSEKGAHPSGGADDVAKKPPPAPQQPPPPPAPHPQQHPQQHPQNQAHGKGGHRGGGGGGGKSSSSSSASAAAAAAAASSSASCSRRLGRALNFLFYLALVAAAAFSGWCVHHVLEEVQQVRRSHQDFSRQREELGQGLQGVEQKVQSLQATFGTFESILRSSQHKQDLTEKAVKQGESEVSRISEVLQKLQNEILKDLSDGIHVVKDARERDFTSLENTVEERLTELTKSINDNIAIFTEVQKRSQKEINDMKAKVASLEESEGNKQDLKALKEAVKEIQTSAKSREWDMEALRSTLQTMESDIYTEVRELVSLKQEQQAFKEAADTERLALQALTEKLLRSEESVSRLPEEIRRLEEELRQLKSDSHGPKEDGGFRHSEAFEALQQKSQGLDSRLQHVEDGVLSMQVASARQTESLESLLSKSQEHEQRLAALQGRLEGLGSSEADQDGLASTVRSLGETQLVLYGDVEELKRSVGELPSTVESLQKVQEQVHTLLSQDQAQAARLPPQDFLDRLSSLDNLKASVSQVEADLKMLRTAVDSLVAYSVKIETNENNLESAKGLLDDLRNDLDRLFVKVEKIHEKV

SEQ ID NO: 44 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 45 - protein, 331 aa, Homo sapiens
MENPSPAAALGKALCALLLATLGAAGQPLGGESICSARALAKYSITFTGKWSQTAFPKQYPLFRPPAQWSSLLGAAHSSDYSMWRKNQYVSNGLRDFAERGEAWALMKEIEAAGEALQSVHAVFSAPAVPSGTGQTSAELEVQRRHSLVSFVVRIVPSPDWFVGVDSLDLCDGDRWREQAALDLYPYDAGTDSGFTFSSPNFATIPQDTVTEITSSSPSHPANSFYYPRLKALPPIARVTLVRLRQSPRAFIPPAPVLPSRDNEIVDSASVPETPLDCEVSLWSSWGLCGGHCGRLGTKSRTRYVRVQPANNGSPCPELEEEAECVPDNCV

SEQ ID NO: 46 - protein, 318 aa, Homo sapiens
MASPGCLLCVLGLLLCGAASLELSRPHGDTAKKPIIGILMQKCRNKVMKNYGRYYIAASYVKYLESAGARVVPVRLDLTEKDYEILFKSINGILFPGGSVDLRRSDYAKVAKIFYNLSIQSFDDGDYFPVWGTCLGFEELSLLISGECLLTATDTVDVAMPLNFTGGQLHSRMFQNFPTELLLSLAVEPLTANFHKWSLSVKNFTMNEKLKKFFNVLTTNTDGKIEFISTMEGYKYPVYGVQWHPEKAPYEWKNLDGISHAPNAVKTAFYLAEFFVNEARKNNHHFKSESEEEKALIYQFSPIYTGNISSFQQCYIFD

SEQ ID NO: 47 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 48 - protein, 655 aa, Homo sapiens
MVAPKSHTDDWAPGPFSSKPQRSQLQIFSSVLQTSLLFLLMGLRASGKDSAPTVVSGILGGSVTLPLNISVDTEIENVIWIGPKNALAFARPKENVTIMVKSYLGRLDITKWSYSLCISNLTLNDAGSYKAQINQRNFEVTTEEEFTLFVYEQLQEPQVTMKSVKVSENFSCNITLMCSVKGAEKSVLYSWTPREPHASESNGGSILTVSRTPCDPDLPYICTAQNPVSQRSSLPVHVGQFCTDPGASRGGTTGETVVGVLGEPVTLPLALPACRDTEKVVWLFNTSIISKEREEAATADPLIKSRDPYKNRVWVSSQDCSLKISQLKIEDAGPYHAYVCSEASSVTSMTHVTLLIYRRLRKPKITWSLRHSEDGICRISLTCSVEDGGNTVMYTWTPLQKEAVVSQGESHLNVSWRSSENHPNLTCTASNPVSRSSHQFLSENICSGPERNTKLWIGLFLMVCLLCVGIFSWCIWKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 49 - protein, 146 aa, Homo sapiens
MTMRSLLRTPFLCGLLWAFCAPGARAEEPAASFSQPGSMGLDKNTVHDQEHIMEHLEGVINKPEAEMSPQELQLHYFKMHDYDGNNLLDGLELSTAITHVHKEEGSEQAPLMSEDELINIIDGVLRDDDKNNDGYIDYAEFAKSLQ

SEQ ID NO: 50 - protein, 427 aa, Homo sapiens
MRHLGAFLFLLGVLGALTEMCEIPEMDSHLVEKLGQHLLPWMDRLSLEHLNPSIYVGLRLSSLQAGTKEDLYLHSLKLGYQQCLLGSAFSEDDGDCQGKPSMGQLALYLLALRANCEFVRGHKGDRLVSQLKWFLEDEKRAIGHDHKGHPHTSYYQYGLGILALCLHQKRVHDSVVDKLLYAVEPFHQGHHSVDTAAMAGLAFTCLKRSNFNPGRRQRITMAIRTVREEILKAQTPEGHFGNVYSTPLALQFLMTSPMRGAELGTACLKARVALLASLQDGAFQNALMISQLLPVLNHKTYIDLIFPDCLAPRVMLEPAAETIPQTQEIISVTLQVLSLLPPYRQSISVLAGSTVEDVLKKAHELGGFTYETQASLSGPYLTSVMGKAAGEREFWQLLRDPNTPLLQGIADYRPKDGETIELRLVSW

SEQ ID NO: 51 - protein, 361 aa, Homo sapiens
MAGGRHRRVVGTLHLLLLVAALPWASRGVSPSASAWPEEKNYHQPAILNSSALRQIAEGTSISEMWQNDLQPLLIERYPGSPGSYAARQHIMQRIQRLQADWVLEIDTFLSQTPYGYRSFSNIISTLNPTAKRHLVLACHYDSKYFSHWNNRVFVGATDSAVPCAMMLELARALDKKLLSLKTVSDSKPDLSLQLIFFDGEEAFLHWSPQDSLYGSRHLAAKMASTPHPPGARGTSQLHGMDLLVLLDLIGAPNPTFPNFFPNSARWFERLQAIEHELHELGLLKDHSLEGRYFQNYSYGGVIQDDHIPFLRRGVPVLHLIPSPFPEVWHTMDDNEENLDESTIDNLNKILQVFVLEYLHL

SEQ ID NO: 52 - protein, 999 aa, Homo sapiens
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 53 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 54 - protein, 99 aa, Homo sapiens
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 55 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 56 - protein, 1060 aa, Homo sapiens
MDSGAGGRRCPEAALLILGPPRMEHLRHSPGPGGQRLLLPSMLLALLLLLAPSPGHATRVVYKVPEEQPPNTLIGSLAADYGFPDVGHLYKLEVGAPYLRVDGKTGDIFTTETSIDREGLRECQNQLPGDPCILEFEVSITDLVQNGSPRLLEGQIEVQDINDNTPNFASPVITLAIPENTNIGSLFPIPLASDRDAGPNGVASYELQAGPEAQELFGLQVAEDQEEKQPQLIVMGNLDRERWDSYDLTIKVQDGGSPPRASSALLRVTVLDTNDNAPKFERPSYEAELSENSPIGHSVIQVKANDSDQGANAEIEYTFHQAPEVVRRLLRLDRNTGLITVQGPVDREDLSTLRFSVLAKDRGTNPKSARAQVVVTVKDMNDNAPTIEIRGIGLVTHQDGMANISEDVAEETAVALVQVSDRDEGENAAVTCVVAGDVPFQLRQASETGSDSKKKYFLQTTTPLDYEKVKDYTIEIVAVDSGNPPLSSTNSLKVQVVDVNDNAPVFTQSVTEVAFPENNKPGEVIAEITASDADSGSNAELVYSLEPEPAAKGLFTISPETGEIQVKTSLDREQRESYELKVVAADRGSPSLQGTATVLVNVLDCNDNDPKFMLSGYNFSVMENMPALSPVGMVTVIDGDKGENAQVQLSVEQDNGDFVIQNGTGTILSSLSFDREQQSTYTFQLKAVDGGVPPRSAYVGVTINVLDENDNAPYITAPSNTSHKLLTPQTRLGETVSQVAAEDFDSGVNAELIYSIAGGNPYGLFQIGSHSGAITLEKEIERRHHGLHRLVVKVSDRGKPPRYGTALVHLYVNETLANRTLLETLLGHSLDTPLDIDIAGDPEYERSKQRGNILFGVVAGVVAVALLIALAVLVRYCRQREAKSGYQAGKKETKDLYAPKPSGKASKGNKSKGKKSKSPKPVKPVEDEDEAGLQKSLKFNLMSDAPGDSPRIHLPLNYPPGSPDLGRHYRSNSPLPSIQLQPQSPSASKKHQVVQDLPPANTFVGTGDTTSTGSEQYSDYSYRTNPPKYPSKQVGQPFQLSTPQPLPHPYHGAIWTEVWE

SEQ ID NO: 57 - protein, 1114 aa, Homo sapiens
MLRGTMTAWRGMRPEVTLACLLLATAGCFADLNEVPQVTVQPASTVQKPGGTVILGCVVEPPRMNVTWRLNGKELNGSDDALGVLITHGTLVITALNNHTVGRYQCVARMPAGAVASVPATVTLANLQDFKLDVQHVIEVDEGNTAVIACHLPESHPKAQVRYSVKQEWLEASRGNYLIMPSGNLQIVNASQEDEGMYKCAAYNPVTQEVKTSGSSDRLRVRRSTAEAARIIYPPEAQTIIVTKGQSLILECVASGIPPPRVTWAKDGSSVTGYNKTRFLLSNLLIDTTSEEDSGTYRCMADNGVGQPGAAVILYNVQVFEPPEVTMELSQLVIPWGQSAKLTCEVRGNPPPSVLWLRNAVPLISSQRLRLSRRALRVLSMGPEDEGVYQCMAENEVGSAHAVVQLRTSRPSITPRLWQDAELATGTPPVSPSKLGNPEQMLRGQPALPRPPTSVGPASPQCPGEKGQGAPAEAPIILSSPRTSKTDSYELVWRPRHEGSGRAPILYYVVKHRKVTNSSDDWTISGIPANQHRLTLTRLDPGSLYEVEMAAYNCAGEGQTAMVTFRTGRRPKPEIMASKEQQIQRDDPGASPQSSSQPDHGRLSPPEAPDRPTISTASETSVYVTWIPRGNGGFPIQSFRVEYKKLKKVGDWILATSAIPPSRLSVEITGLEKGTSYKFRVRALNMLGESEPSAPSRPYVVSGYSGRVYERPVAGPYITFTDAVNETTIMLKWMYIPASNNNTPIHGFYIYYRPTDSDNDSDYKKDMVEGDKYWHSISHLQPETSYDIKMQCFNEGGESEFSNVMICETKARKSSGQPGRLPPPTLAPPQPPLPETIERPVGTGAMVARSSDLPYLIVGVVLGSIVLIIVTFIPFCLWRAWSKQKHTTDLGFPRSALPPSCPYTMVPLGGLPGHQASGQPYLSGISGRACANGIHMNRGCPSAAVGYPGMKPQQHCPGELQQQSDTSSLLRQTHLGNGYDPQSHQITRGPKSSPDEGSFLYTLPDDSTHQLLQPHHDCCQRQEQPAAVGQSGVRRAPDSPVLEAVWDPPFHSGPPCCLGLVPVEEVDSPDSCQVSGGDWCPQHPVGAYVGQEPGMQLSPGPLVRVSFETPPLTI

SEQ ID NO: 58 - protein, 525 aa, Homo sapiens
MRVFCVGLLLFSVTWAAPTFQPQTEKTKQSCVEEQRQEEKNKDNIGFHHLGKRINQELSSKENIVQERKKDLSLSEASENKGSSKSQNYFTNRQRLNKEYSISNKENTHNGLRMSIYPKSTGNKGFEDGDDAISKLHDQEEYGAALIRNNMQHIMGPVTAIKLLGEENKENTPRNVLNIIPASMNYAKAHSKDKKKPQRDSQAQKSPVKSKSTHRIQHNIDYLKHLSKVKKIPSDFEGSGYTDLQERGDNDISPFSGDGQPFKDIPGKGEATGPDLEGKDIQTGFAGPSEAESTHLDTKKPGYNEIPEREENGGNTIGTRDETAKEADAVDVSLVEGSNDIMGSTNFKELPGREGNRVDAGSQNAHQGKVEFHYPPAPSKEKRKEGSSDAAESTNYNEIPKNGKGSTRKGVDHSNRNQATLNEKQRFPSKGKSQGLPIPSRGLDNEIKNEMDSFNGPSHENIITHGRKYHYVPHRQNNSTRNKGMPQGKGSWGRQPHSNRRFSSRRRDDSSESSDSGSSSESDGD

SEQ ID NO: 59 - protein, 832 aa, Homo sapiens
MKPPGSSSRQPPLAGCSLAGASCGPQRGPAGSVPASAPARTPPCRLLLVLLLLPPLAASSRPRAWGAAAPSAPHWNETAEKNLGVLADEDNTLQQNSSSNISYSNAMQKEITLPSRLIYYINQDSESPYHVLDTKARHQQKHNKAVHLAQASFQIEAFGSKFILDLILNNGLLSSDYVEIHYENGKPQYSKGGEHCYYHGSIRGVKDSKVALSTCNGLHGMFEDDTFVYMIEPLELVHDEKSTGRPHIIQKTLAGQYSKQMKNLTMERGDQWPFLSELQWLKRRKRAVNPSRGIFEEMKYLELMIVNDHKTYKKHRSSHAHTNNFAKSVVNLVDSIYKEQLNTRVVLVAVETWTEKDQIDITTNPVQMLHEFSKYRQRIKQHADAVHLISRVTFHYKRSSLSYFGGVCSRTRGVGVNEYGLPMAVAQVLSQSLAQNLGIQWEPSSRKPKCDCTESWGGCIMEETGVSHSRKFSKCSILEYRDFLQRGGGACLFNRPTKLFEPTECGNGYVEAGEECDCGFHVECYGLCCKKCSLSNGAHCSDGPCCNNTSCLFQPRGYECRDAVNECDITEYCTGDSGQCPPNLHKQDGYACNQNQGRCYNGECKTRDNQCQYIWGTKAAGSDKFCYEKLNTEGTEKGNCGKDGDRWIQCSKHDVFCGFLLCTNLTRAPRIGQLQGEIIPTSFYHQGRVIDCSGAHVVLDDDTDVGYVEDGTPCGPSMMCLDRKCLQIQALNMSSCPLDSKGKVCSGHGVCSNEATCICDFTWAGTDCSIRDPVRNLHPPKDEGPKGPSATNLIIGSIAGAILVAAIVLGGTGWGFKNVKKRRFDPTQQGPI

SEQ ID NO: 60 - protein, 689 aa, Homo sapiens
MKPPAACAGDMADAASPCSVVNDLRWDLSAQQIEERTRELIEQTKRVYDQVGTQEFEDVSYESTLKALADVEVTYTVQRNILDFPQHVSPSKDIRTASTEADKKLSEFDVEMSMREDVYQRIVWLQEKVQKDSLRPEAARYLERLIKLGRRNGLHLPRETQENIKRIKKKLSLLCIDFNKNLNEDTTFLPFTLQELGGLPEDFLNSLEKMEDGKLKVTLKYPHYFPLLKKCHVPETRRKVEEAFNCRCKEENCAILKELVTLRAQKSRLLGFHTHADYVLEMNMAKTSQTVATFLDELAQKLKPLGEQERAVILELKRAECERRGLPFDGRIRAWDMRYYMNQVEETRYCVDQNLLKEYFPVQVVTHGLLGIYQELLGLAFHHEEGASAWHEDVRLYTARDAASGEVVGKFYLDLYPREGKYGHAACFGLQPGCLRQDGSRQIAIAAMVANFTKPTADAPSLLQHDEVETYFHEFGHVMHQLCSQAEFAMFSGTHVERDFVEAPSQMLENWVWEQEPLLRMSRHYRTGSAVPRELLEKLIESRQANTGLFNLRQIVLAKVDQALHTQTDADPAEEYARLCQEILGVPATPGTNMPATFGHLAGGYDAQYYGYLWSEVYSMDMFHTRFKQEGVLNSKVGMDYRSCILRPGGSEDASAMLRRFLGRDPKQDAFLLSKGLQVGGCEPEPQVC

SEQ ID NO: 61 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 62 - protein, 485 aa, Homo sapiens
MPAMVEKGPEVSGKRRGRNNAAASASAAAASAAASAACASPAATAASGAAASSASAAAASAAAAPNNGQNKSLAAAAPNGNSSSNSWEEGSSGSSSDEEHGGGGMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARKQKREREESEDELEEANGNNPIDIEVDQNKESKKEVPPTETVPQVKKEKHSTQAKNRAKRKPPKGMFLSQEDVEAVSANATAATTVLRQLDMELVSVKRQIQNIKQTNSALKEKLDGGIEPYRLPEVIQKCNARWTTEEQLLAVQAIRKYGRDFQAISDVIGNKSVVQVKNFFVNYRRRFNIDEVLQEWEAEHGKEETNGPSNQKPVKSPDNSIKMPEEEDEAPVLDVRYASAS

SEQ ID NO: 63 - protein, 448 aa, Homo sapiens
MAHVGDCTQTPWLPVLVVSLMCSARAEYSNCGENEYYNQTTGLCQECPPCGPGEEPYLSCGYGTKDEDYGCVPCPAEKFSKGGYQICRRHKDCEGFFRATVLTPGDMENDAECGPCLPGYYMLENRPRNIYGMVCYSCLLAPPNTKECVGATSGASANFPGTSGSSTLSPFQHAHKELSGQGHLATALIIAMSTIFIMAIAIVLIIMFYILKTKPSAPACCTSHPGKSVEAQVSKDEEKKEAPDNVVMFSEKDEFEKLTATPAKPTKSENDASSENEQLLSRSVDSDEEPAPDKQGSPELCLLSLVHLAREKSATSNKSAGIQSRRKKILDVYANVCGVVEGLSPTELPFDCLEKTSRMLSSTYNSEKAVVKTWRHLAESFGLKRDEIGGMTDGMQLFDRISTAGYSIPELLTKLVQIERLDAVESLCADILEWAGVVPPASQPHAAS

SEQ ID NO: 64 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 65 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 66 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 67 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 68 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 69 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 70 - protein, 90 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVE

SEQ ID NO: 71 - protein, 211 aa, Homo sapiens [sequence omitted]

SEQ ID NO: 72 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 73 - protein, 375 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSASGFSCAGRYSENISRQTSETADNDNASSFTM